(19) United States
(12) Patent Application Publication
(10) Pub. No.: US 2025/0122280 A1
(43) Pub. Date: Apr. 17, 2025

(54) ANTI-Ly6E ANTIBODIES, IMMUNOCONJUGATES, AND USES THEREOF

(71) Applicant: Genentech, Inc., South San Francisco, CA (US)

(72) Inventors: Nicholas John Agard, San Mateo, CA (US); Thomas Harden Pillow, Millbrae, CA (US); Josefa Dela Cruz Chuh, South San Francisco, CA (US); Mary Ann T. Go, South San Francisco, CA (US); Katherine R. Kozak, South San Francisco, CA (US); Zhonghua Lin, South San Francisco, CA (US); Dhaya Seshasayee, South San Francisco, CA (US); Shang-Fan Yu, South San Francisco, CA (US); James Thomas Koerber, San Mateo, CA (US); Yonglian Sun, Foster City, CA (US)

(73) Assignee: Genentech, Inc., South San Francisco, CA (US)

(21) Appl. No.: 18/927,265

(22) Filed: Oct. 25, 2024

Related U.S. Application Data
(63) Continuation of application No. PCT/US23/66486, filed on May 2, 2023.
(60) Provisional application No. 63/337,945, filed on May 3, 2022.
(60) Provisional application No. 63/492,297, filed on Mar. 27, 2023.

(51) Int. Cl. C07K 16/28; A61K 45/06; A61K 47/68; A61K 51/10; A61P 35/00; G01N 33/574; G01N 33/68

(52) U.S. Cl. CPC C07K 16/28; A61K 45/06; A61K 47/68035; A61K 47/6849; A61K 51/1093; A61P 35/00; C07K 16/2809; G01N 33/57492; G01N 33/6893; A61K 2123/00; C07K 2317/24; G01N 2333/705

(57) ABSTRACT

The invention provides anti-Ly6E antibodies and immunoconjugates, and methods of using the same.

### CROSS-REFERENCE TO RELATED APPLICATIONS

This application is a continuation of International Application No. PCT/US2023/066486, filed May 2, 2023, which claims the benefit of priority of US Provisional Application Nos. 63/337,945, filed May 3, 2022, and 63/492,297, filed Mar. 27, 2023, each of which is incorporated by reference herein in their entirety for any purpose.

### SEQUENCE LISTING

The present application contains a Sequence Listing, which has been submitted electronically in XML format. Said XML copy, created on Oct. 21, 2024, is named “01146-0114-00US-ST26.xml” and is 46,920 bytes in size. The information in the electronic format of the sequence listing is incorporated herein by reference herein in its entirety.

### FIELD

The present invention relates to anti-Ly6E antibodies and immunoconjugates, and methods of using the same.

### BACKGROUND

Lymphocyte antigen 6 complex, locus E (Ly6E), also known as retinoic acid induced gene E (RIG-E) and stem cell antigen 2 (SCA-2). It is a GPI linked, 131 amino acid length, ˜8.4 kDa protein of unknown function with no known binding partners. It was initially identified as a transcript expressed in immature thymocyte, thymic medullary epithelial cells in mice. Mao et al., “RIG-E, a human homolog of the murine Ly-6 family, is induced by retinoic acid during the differentiation of acute promyelocytic leukemia cell,” Proc. Natl. Acad. Sci. U.S.A. 93:5910-5914 (1996).

There is a need in the art for agents that target Ly6E for the diagnosis and treatment of Ly6E-associated conditions, such as cancer. The invention fulfills that need and provides other benefits.

### SUMMARY

The invention provides anti-Ly6E antibodies, immunoconjugates, and methods of using the same.

In some embodiments, the disclosure provides an isolated antibody that binds to Ly6E, wherein the antibody comprises (i) HVR-L1 comprising the amino acid sequence of SEQ ID NO:7, (ii) HVR-L2 comprising the amino acid sequence of SEQ ID NO:8, (iii) HVR-L3 comprising the amino acid sequence of SEQ ID NO:9, (iv) HVR-H1 comprising the amino acid sequence of SEQ ID NO:10, v) HVR-H2 comprising the amino acid sequence of SEQ ID NO:11, and (vi) HVR-H3 comprising the amino acid sequence of SEQ ID NO: 12.

In some embodiments, the antibody is a monoclonal antibody. In some embodiments, the antibody is a humanized or chimeric antibody. In some embodiments, the antibody is an antibody fragment.

In some embodiments, the antibody comprises (a) a VH sequence having at least 95% sequence identity to SEQ ID NO:32; (b) a VL sequence having at least 95% sequence identity to the amino acid sequence of SEQ ID NO:31; or (c) a VH sequence as in (a) and a VL sequence as in (b). In some embodiments, the antibody comprises a VH sequence of SEQ ID NO:32 and a VL sequence of SEQ ID NO: 31.

In some embodiments, the disclosure provides an isolated antibody comprising a VH sequence SEQ ID NO:32 and a VL sequence of SEQ ID NO:31.

In some embodiments, the antibody is an IgG1, IgG2a, IgG2b, IgG3, or IgG4 antibody.

In some embodiments, the antibody comprises a heavy chain comprising an amino acid sequence selected from SEQ ID NOs: 5 and a light chain comprising an amino acid sequence selected from SEQ ID NOs: SEQ ID NO:3, SEQ ID NO: 17, SEQ ID NO: 19, or SEQ ID NO: 29. In some embodiments, the antibody comprises a heavy chain comprising the amino acid sequence of SEQ ID NO: 5 and a light chain comprising the amino acid sequence of SEQ ID NO: 3. In some embodiments, the disclosure provides an isolated antibody that binds to Ly6E, wherein the antibody comprises a heavy chain comprising the amino acid sequence of SEQ ID NO: 5 and a light chain comprising the amino acid sequence of SEQ ID NO: 3.

In some embodiments, the antibody is a multispecific antibody. In some embodiments, the multispecific antibody is a bispecific antibody. In some embodiments, the multispecific antibody binds Ly6E and CD3.

In some embodiments, the disclosure provides an isolated nucleic acid encoding the antibody. In some embodiments, the disclosure provides an expression vector comprising the nucleic acid encoding the antibody. In some embodiments, the disclosure provides a host cell comprising the nucleic acid or the expression vector. In some embodiments, the disclosure provides a host cell that expresses the antibody. In some embodiments, the disclosure provides a method of producing an antibody comprising culturing the host cell so that the antibody is produced.

In some embodiments, the disclosure provides an immunoconjugate comprising the antibody and a cytotoxic agent. In some embodiments, the immunoconjugate has the formula Ab-(L-D)p, wherein: (a) Ab is an antibody provided herein; (b) L is a linker; (c) D is a pyrrolobenzodiazepine; and (d) p ranges from 1-8. In some embodiments, D is a pyrrolobenzodiazepine of Formula A:

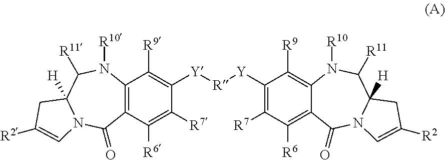

wherein:
R² is of formula II:

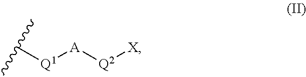

wherein A is a C₅₋₇ aryl group, X is selected from the group consisting of: OH, SH, CO₂H, COH, N═C═O, NHR^{N}, and (OC₂H₄)ₘOCH₃, wherein R^{N} is selected from the group consisting of H and C₁₋₄ alkyl, and wherein m is an integer from 1 to 3, and either:
(i) Q¹ is a single bond, and Q² is selected from a single bond and —Z—(CH₂)ₙ—, wherein Z is a single bond, O, S, or NH and n is an integer from 1 to 3; or
(ii) Q¹ is —CH═CH—, and Q² is a single bond;
R^{2′} is a C₅₋₁₀ aryl group, optionally substituted by one or more substituents selected from the group consisting of halo, nitro, cyano, ether, C₁₋₇ alkyl, C₃₋₇ heterocyclyl, and bis-oxy-C₁₋₃ alkylene;
R⁶ and R⁹ are independently selected from H, R, OH, OR, SH, SR, NH₂, NHR, NRR′, nitro,
Me₃Sn and halo;
R⁷ is selected from the group consisting of H, R, OH, OR, SH, SR, NH₂, NHR, NHRR′, nitro, Me₃Sn, and halo;
where R and R′ are independently selected from optionally substituted C₁₋₁₂ alkyl, C₃₋₂₀ heterocyclyl and C₅₋₂₀ aryl groups;
either:
   (a) R¹⁰ is H, and R¹¹ is OH or OR^{A}, wherein R^{A} is C₁₋₄ alkyl;
   (b) R¹⁰ and R¹¹ form a nitrogen-carbon double bond between the nitrogen and carbon atoms to which they are bound; or
   (c) R¹⁰ is H and R₁₁ is SO_{z}M, wherein z is 2 or 3 and M is a monovalent pharmaceutically acceptable cation;
R″ is a C₃₋₁₂ alkylene group, which chain may be interrupted by one or more heteroatoms independently selected from the group consisting of O, S, and NH, and/or one or more aromatic rings independently selected from the group consisting of benzene or pyridine;
Y is O, S, or NH;
R^{6′}, R^{7′}, and R^{9′} are selected from the same groups as R⁶, R⁷ and R⁹ respectively, and R^{10′} and R^{11′} are the same as R¹⁰ and R¹¹ respectively, wherein if R¹¹ and R^{11′} are SO_{z}M, M may represent a divalent pharmaceutically acceptable cation; and
the point of attachment to the linker L is through R² or R^{2′}.

In some embodiments, D has the structure:

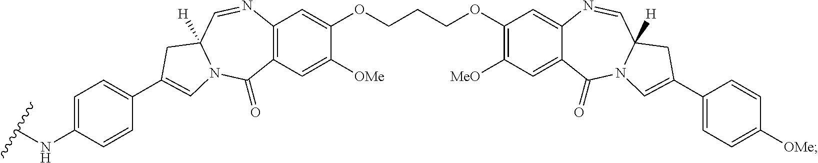 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

wherein the squiggly line indicates the point of attachment to the linker L.

In some embodiments, (L-D) has the structure:

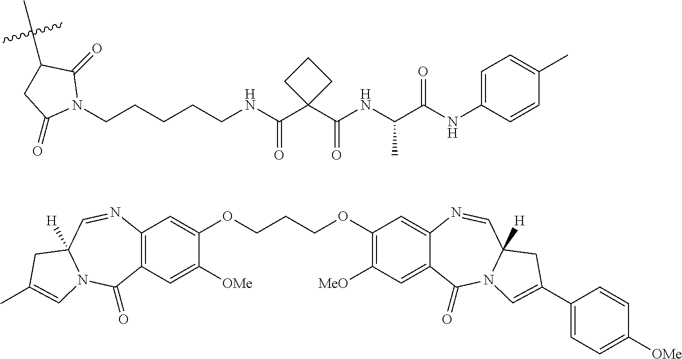

wherein the squiggly line indicates the point of attachment to the protein.

In some embodiments, p ranges from 1.5-5 or 1.5-6 or 1.5-4 or 2-3.

In some embodiments, the disclosure provides a pharmaceutical formulation comprising the immunoconjugate and a pharmaceutically acceptable carrier. In some embodiments, the pharmaceutical formulation further comprises an additional therapeutic agent.

In some embodiments, the disclosure provides a method of treating an individual having an Ly6E-positive cancer, the method comprising administering to the individual an effective amount of the immunoconjugate or the pharmaceutical formulation. In some embodiments, the Ly6E-positive cancer is selected from a breast cancer, pancreatic cancer, colon cancer, colorectal cancer, melanoma, ovarian cancer, non-small cell lung cancer, or gastric cancer. breast cancer

In some embodiments, the method further comprises administering an additional therapeutic agent to the individual. In some embodiments, the additional therapeutic agent is a platinum complex.

In some embodiments, the disclosure provides a method of inhibiting proliferation of an Ly6E-positive cell, the method comprising exposing the cell to the immunoconjugate under conditions permissive for binding of the immunoconjugate to Ly6E on the surface of the cell, thereby inhibiting proliferation of the cell. In some embodiments, the cell is a breast, pancreatic, colon, colorectal, melanoma, ovarian non-small cell lung or gastric cancer cell.

In some embodiments, the disclosure provides an antibody provided herein conjugated to a label. In some embodiments, the label is a positron emitter. In some embodiments, the positron emitter is 89Zr.

In some embodiments, the disclosure provides a method of detecting human Ly6E in a biological sample comprising contacting the biological sample with the anti-Ly6E antibody under conditions permissive for binding of the anti-Ly6E antibody to a naturally occurring human Ly6E, and detecting whether a complex is formed between the anti-Ly6E antibody and a naturally occurring human Ly6E in the biological sample. In some embodiments, the biological sample is a breast cancer sample, a pancreatic cancer sample, a colon cancer sample, a colorectal cancer sample, melanoma cancer sample, ovarian cancer sample, a non-small cell lung cancer sample, or a gastric cancer sample.

In some embodiments, the disclosure provides a method for detecting a Ly6E-positive cancer comprising: (i) administering a labeled anti-Ly6E antibody to a subject having or suspected of having a Ly6E-positive cancer, wherein the labeled anti-Ly6E antibody comprises an anti-Ly6E antibody provided herein; and (ii) detecting the labeled anti-Ly6E antibody in the subject, wherein detection of the labeled anti-Ly6E antibody indicates a Ly6E-positive cancer in the subject. In some embodiments, the labeled anti-Ly6E antibody comprises an anti-Ly6E antibody conjugated to a positron emitter. In some embodiments, the positron emitter is ⁸⁹Zr.

### BRIEF DESCRIPTION OF THE FIGURES

FIG. 1 shows the structure of the (maleimide-sq-ala)-PBD linker-drug, prior to conjugation to an anti-Ly6E antibody.

FIGS. 2A-2C show binding of chimeric anti-Ly6E antibodies to cells, through flow cytometry analysis of chimeric anti-Ly6E and 9B12 binding to several cell lines known to express Ly6E (FIG. 2A Kuramochi cells, FIG. 2B NCI-H1781, FIG. 2C HCC1569x2).

FIGS. 3A and 3B show binding of chimeric and humanized anti-Ly6E antibodies to cells, through flow cytometry analysis of chimeric and humanized anti-Ly6E along with 9B12 and an isotype control binding to several cell lines known to express Ly6E.

FIGS. 4A-4C. FIG. 4A shows a peptide map of CDR-L3 of humanized anti-Ly6E (positions 61-102 of SEQ ID NO: 17). In FIGS. 4B and 4C, humanized anti-Ly6E was subjected to zero or two weeks of thermal stress and then trypsinized prior to analysis by LC-MS/MS. Peaks for native, isomerized and succinimide-containing peptides were integrated at retention times of 30.6-30.7, 31.1, and 32-32.1 minutes respectively.

FIG. 5 shows cell binding of anti-Ly6E Fab variants. Fabs of humanized anti-Ly6E and variants intended to remove a site of aspartic acid isomerization were analyzed for binding to NCl—H1781 cells by flow cytometry.

FIGS. 6A-6B show in vitro activity of anti-Ly6E ADC with a seco-CBI-dimer payload. Anti-Ly6E-SN36325 conjugates induce superior cell death to untargeted ADCs or 9B12 conjugates.

FIG. 7 shows in vitro activity of anti-Ly6E ADC with a PBD payload. Anti-Ly6E-SGD-1882 conjugates induce superior cell death to 9B12 conjugates.

FIGS. 8A and 8B show in vivo efficacy of anti-Ly6E ADCs in HCC1569X2 tumor xenograft model. Growth of breast model HCC1569X2 was assessed following a single administration of Vehicle or ADC. Conjugates were administered once on day 0, and individual tumor volumes and body weight changes are plotted. Numbers next to traces indicate dose of each conjugate (in mg/kg) that was administered once IV at day 0. Cubic spline fitted tumor volumes are plotted for each treatment group (n=5/group). Cubic spline fits for vehicle group (dashed blue line) and treatment group (solid line) are included for reference.

FIGS. 9A-9F show in vitro activity of humanized anti-Ly6E bispecific antibodies. Anti-Ly6E/anti-CD3 bispecific T-cell engagers induce cell death in a number of cell lines. Anti-Ly6E bispecifics show enhanced activity over 9B12 bispecifics.

FIGS. 10A-10B show that chimeric (Ch) Ly6E 4B10 and 3A3 show >400-fold potency difference from non-target control (e.g., isotype controls anti-gD and anti-CD22) compared to only 9-fold with 9B12 in cell line NCl—H1781 after a 5 day anti-proliferation assay. In FIG. 10B, TDC Ab-SN36325 refers to ThioMab Drug Conjugate conjugated to Auckland SN36325 drug, and CNJ designates the conjugation lot.

FIGS. 11A-B show that chimeric (Ch) Ly6E 3A3 shows highest potency against cell line HCC1569x2 despite overall weaker potency with partial killing in a 5-day anti-proliferation assay.

FIGS. 12A-D show that the top antibody binders using imaging at 1 and 4 hours at 4° C. have similar ranking as with FACS binding. FIG. 12A shows that 3A3 has the highest average intensity. FIG. 12B-12D show the highest Median Fluorescence Intensity (MFI) per concentration (μg/ml) of 3A3 (3.A3.hlgG1) in Kuramochi (FIG. 12B), NCl—H1781 (FIG. 12C), and HCC-1569x2 (FIG. 12D) cell lines.

FIG. 13 shows that the amount of 3A3 (3.A3.hlgG1) colocalization in lysosome (per cell) after 3 hours is significantly higher compared to antibodies 1D5.3, 2.5G6, 9B12, and negative control. PE—pulse exposure (washed off antibody), CE—continuous exposure.

FIG. 14 shows that the rate of internalization for 9B12 (Ly6E9B12NKD) was faster compared to 3A3 (3.A3.hlgG1) but the higher binding of 3A3 leads to durable efficacy as seen in in vivo studies. PE—pulse exposure.

FIG. 15 shows fold change of Ly6E from non-target CD22 in different extracellular matrix (ECM), such as all ECM, GELTREX® (GIBCO™), periodontal ligament (PDL), fibronectin, and phosphate buffered saline (PBS).

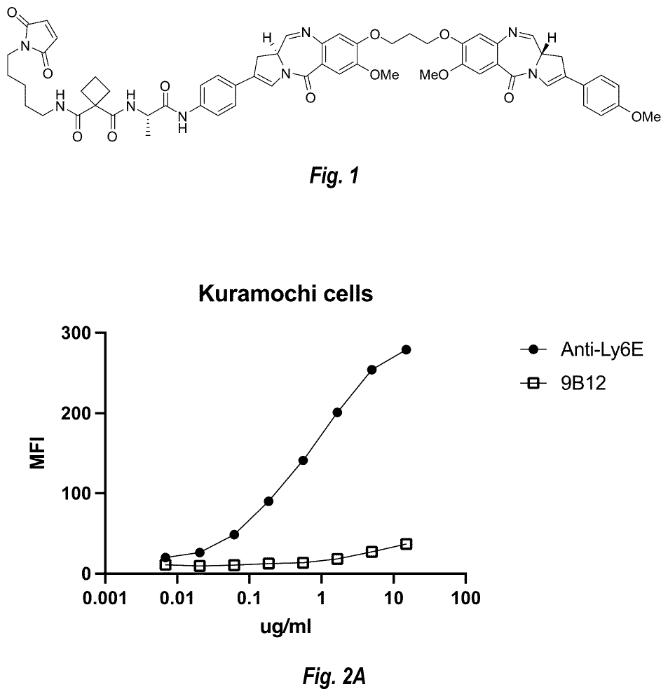

### DETAILED DESCRIPTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-Ly6E antibody” and “an antibody that binds to Ly6E” refer to an antibody that is capable of binding Ly6E with sufficient affinity such that the antibody is useful as a diagnostic and/or therapeutic agent in targeting Ly6E. In one embodiment, the extent of binding of an anti-Ly6E antibody to an unrelated, non-Ly6E protein is less than about 10% of the binding of the antibody to Ly6E as measured, e.g., by a radioimmunoassay (RIA) or by scatchard analysis or by surface plasmon resonance, such as, for example, BIAcore™. In certain embodiments, an antibody that binds to Ly6E has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g., from 10⁻⁸ M to 10⁻¹³ M, e.g., from 10⁻⁹ M to 10⁻¹³ M). In certain embodiments, an anti-Ly6E antibody binds to an epitope of Ly6E that is conserved among Ly6E from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “antibody drug conjugate” (ADC) as used herein is equivalent to the term “immunoconjugate”.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include a cancer that over-expresses Ly6E, which may include, for example, breast cancer and/or metastatic breast cancer, including Her2 negative breast cancers and/or triple negative breast cancers, pancreatic cancer, colon cancer, colorectal cancer, melanoma, ovarian cancer, non-small cell lung cancer (either squamous and/or non-squamous), gastric cancer, squamous cell cancer, small-cell lung cancer, adenocarcinoma of the lung, squamous carcinoma of the lung, cancer of the peritoneum, hepatocellular cancer, gastrointestinal cancer, glioma, cervical cancer, liver cancer, bladder cancer, hepatoma, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG1 isotype. In certain aspects, the antibody is of the IgG1 isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g., described by Kabat, E. A., et al., Sequences of Proteins of Immunological Interest, 5th ed., Public Health Service, National Institutes of Health, Bethesda, MD (1991) (see also, e.g., Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “epitope” refers to the particular site on an antigen molecule to which an antibody binds.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent. An immunoconjugate is equivalent to the term “antibody drug conjugate” (ADC).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-Ly6E antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “Ly6E,” as used herein, refers to any native, mature Ly6E which results from processing of a Ly6E precursor protein in a cell. The term includes Ly6E from any vertebrate source, including mammals such as primates (e.g., humans and cynomolgus or rhesus monkeys) and rodents (e.g., mice and rats), unless otherwise indicated. The term also includes naturally occurring variants of Ly6E, e.g., splice variants or allelic variants. The amino acid sequence of an exemplary human Ly6E precursor protein, with signal sequence (amino acids 1-20=signal sequence) is shown in SEQ ID NO: 1. The amino acid sequence of an exemplary mature human Ly6E is shown in SEQ ID NO: 24. The sequence for amino acids 1-131 of an exemplary cynomologous monkey Ly6E is shown in SEQ ID NO: 2. The amino acid sequence of an exemplary mature cynomologous Ly6E is shown in SEQ ID NO: 25. The amino acid sequence for an exemplary rat Ly6E precursor (with signal sequence, amino acids 1-26) and mature sequences are shown in SEQ ID NOs: 23 and 28, respectively. The amino acid sequences for exemplary mouse Ly6E precursor (with signal sequence, amino acids 1-26) and mature sequences are shown in SEQ ID NOs: 22 and 27, respectively.

The term “Ly6E-positive cancer” refers to a cancer comprising cells that express Ly6E on their surface. For the purposes of determining whether a cell expresses Ly6E on the surface, Ly6E mRNA expression is considered to correlate to Ly6E expression on the cell surface. In some embodiments, expression of Ly6E mRNA is determined by a method selected from in situ hybridization and RT-PCR (including quantitative RT-PCR). Alternatively, expression of Ly6E on the cell surface can be determined, for example, using antibodies to Ly6E in a method such as immunohistochemistry, FACS, etc. In some embodiments, a Ly6E-positive cancer means a breast cancer, metastatic breast cancer, including Her2 negative breast cancers and/or triple negative breast cancers, pancreatic cancer, colon cancer, colorectal cancer, melanoma, ovarian cancer, non-small cell lung cancer (either squamous and/or non-squamous), or gastric cancer, each of which that exhibits a high level of Ly6E expression.

The term “Ly6E-positive cell” refers to a cancer cell that expresses Ly6E on its surface.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “platinum complex” as used herein refers to anti-cancer chemotherapy drugs such as, for example, but not limited to, cisplatin, oxaliplatin, carboplatin, iproplatin, satraplatin, CI-973, AZ0473, DWA2114R, nedaplatin, and sprioplatin, which exert efficacy against tumors based on their ability to covalently bind to DNA.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al. Kuby Immunology, 6^{th} ed., W. H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

With respect to Formula A, the term “C₁₋₁₂ alkyl” as used in Formula A refers to a monovalent moiety obtained by removing a hydrogen atom from a carbon atom of a hydrocarbon compound having from 1 to 12 carbon atoms, which may be aliphatic or alicyclic, and which may be saturated or unsaturated (e.g., partially unsaturated, fully unsaturated), but not aromatic. Accordingly, the term “alkyl” includes the sub-classes alkenyl, alkynyl, cycloalkyl, etc. Examples of saturated alkyl groups may include, but are not limited to methyl, ethyl, propyl, butyl, pentyl, hexyl, and heptyl. Examples of saturated linear alkyl groups include, but are not limited to, methyl, ethyl, n-propyl (C₃), n-butyl (C₄), n-pentyl (amyl) (C₅), n-hexyl (C₆) and n-heptyl. Examples of saturated branched alkyl groups include iso-propyl (C₃), iso-butyl (C₄), sec-butyl (C₄), tert-butyl (C₄), iso-pentyl (C₅), and neo-pentyl (C₅). An alkenyl group as used herein, with respect to Formula A, refers to an alkyl group having one or more carbon-carbon double bonds. Examples of alkenyl groups may include, but are not limited to, ethenyl, 1-propenyl, 2-propenyl, isopropenyl, butenyl, pentenyl, and hexenyl. In some embodiments, the C₁₋₁₂ alkyl of Formula A is a saturated, acyclic alkyl.

In other uses herein, “alkyl” is C₁-C₁₂ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃) CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

The term “C₃₋₁₂ alkylene”, as used herein and particularly with respect to Formula A, refers to a bidentate moiety obtained by removing two hydrogen atoms from a hydrocarbon compound having from 3 to 12 carbon atoms (unless otherwise specified), which may be aliphatic or alicyclic, and which may be saturated, partially unsaturated, or fully unsaturated (but not aromatic). The two hydrogen atoms may be removed from the same carbon atom, or one from each of two different carbon atoms. Accordingly, the term “alkylene” includes the sub-classes alkenylene, alkynylene, cycloalkylene, etc. Examples of linear saturated C₃₋₁₂ alkylene groups may include, but are not limited to, —(CH₂)ₙ— where n is an integer from 3 to 12, such as —CH₂CH₂CH₂—, —CH₂CH₂CH₂CH₂—, —CH₂CH₂CH₂CH₂CH₂—, and —CH₂CH₂CH₂CH-2CH₂CH₂CH₂—. Examples of branched saturated C₃₋₁₂ alkylene groups may include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—. Examples of linear partially unsaturated C₃₋₁₂ alkylene groups (C₃₋₁₂ alkenylene, and alkynylene groups) may include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—. Examples of branched partially unsaturated C₃₋₁₂ alkylene groups (C₃₋₁₂ alkenylene and alkynylene groups) may include, but are not limited to, —C(CH₃)═CH—, —C(CH₃)═CH—CH₂—, —CH═CH—CH(CH₃)— and —CsC—CH(CH₃)—. Examples of alicyclic saturated C₃₋₁₂ alkylene groups (C₃₋₁₂ cycloalkylenes) include, but are not limited to, cyclopentylene, and cyclohexylene. Examples of alicyclic partially unsaturated C₃₋₁₂ alkylene groups may include, but are not limited to, cyclopentenylene and cyclohexenylene. In some embodiments of Formula A, C₃₋₁₂ alkylene refers to a straight chain, saturated hydrocarbon group of the formula —(CH₂)₃₋₁₂—, examples of which include propylene, butylene, pentylene, hexylene, heptylene, ocytylene, nonylene, decalene, undecalene, and dodecalene.

The term “C₅₋₂₀ aryl”, as used herein with respect to Formula A, refers to a monovalent moiety obtained by removing a hydrogen atom from an aromatic ring atom of an aromatic compound, which moiety has from 3 to 20 ring atoms. In some embodiments, each aryl ring has from 5 to 7 ring atoms. The prefixes, e.g., C₃₋₂₀, C₅₋₇, C₅₋₆, etc., refer to the number of ring atoms, or range of number of ring atoms, whether carbon atoms or heteroatoms. For example, the term “C₅₋₆ aryl” as used herein, refers to an aryl group having 5 or 6 ring atoms. In some embodiments all ring atoms are carbon atoms, as in a “carboaryl groups”. Such carboaryl groups may include, but are not limited to, those derived from benzene (i.e., phenyl), naphthalene, azulene, anthracene, phenanthrene, naphthacene, and pyrene. In some embodiments, the aryl comprises fused rings wherein at least one of the fused rings is an aromatic ring, such as, for example, groups derived from indane, indene, isoindene, tetraline, acenaphthene, fluorene, phenalene, acephenanthrene, and aceanthrene. In other embodiments, the ring atoms may include one or more heteroatoms, such as in “heteroaryl groups”. Examples of monocyclic heteroaryl groups may include, but are not limited to, those derived from pyrrole, pyridine, furan. Thiophene, oxazole, isoxazole, isoxazine, oxadiazole, thiazole, isothiazole, and triazole. In some embodiments, the heteroaryl comprises fused rings, wherein at least one of the rings comprises a ring heteroatom, such as groups derived from benzofuran, isobenzofuran, isoindole, indolizine, indoline, isoindoline, purine, benzimidazole, indazole, and benzoxazole. In some embodiments, with respect to Formula A, the C₅₋₂₀ aryl is a C₆-C₂₀ carboaryl group.

“Substituted alkyl,” “substituted aryl,” and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, —PO⁻₃, —PO₃H₂, —C(═O)R, —C(═O)X, —C(═S)R, —CO₂R, —CO₂⁻, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₁₈ alkyl, C₆-C₂₀ aryl, C₃-C₁₄ heterocyclyl, protecting group or prodrug moiety. Alkylene, alkenylene, and alkynylene groups as described above may also be similarly substituted.

The term “C₃₋₂₀ heterocyclyl” as used herein, particularly with respect to Formula A, refers to a monovalent moiety obtained by removing a hydrogen atom from a ring atom of a heterocyclic compound, which moiety has from 3 to 20 ring atoms, of which from 1 to 10 are ring heteroatoms. In some embodiments, each ring has from 3 to 7 ring atoms, of which from 1 to 4 are ring heteroatoms. In some embodiments, each heteroatom is independently selected from the group consisting of O, N, and S. When referring to a heterocyclyl, the prefixes (e.g., C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, refers to a heterocyclyl group having 5 or 6 ring atoms, whether those ring atoms are carbon or heteroatoms. Examples of monocyclic heterocyclyl groups may include, but are not limited to, those derived from aziridine, azetidine, pyrrolidine, pyrroline, piperidine, dihydropyridine, tetrahydropyridine, azepine, oxirane, oxetane, oxolane (tetrahydrofuran), oxole, oxane, dihydropyran, pyran, dioxane, imidazolidine, pyrazolidine, imidazoline, and pyrazoline. In some embodiments, the heterocyclyl group with respect to Formula A is a saturated, mono or bicyclic ring comprising from 3 to 10 ring atoms, of which from 1 to 3 are ring heteroatoms independently selected from the group consisting of O and N.

As used herein, particularly with respect to Formula A, the term “ether” refers to the group —OR, wherein R is an ether substituent, for example, a C₁₋₇alkyl group (which also may be referred to as a C₁₋₇ alkoxy group), a C₃₋₂₀ heterocyclyl group (which also may be referred to as a C₃₋₂₀ heterocyclyloxy group), or a C₅₋₂₀ aryl group (which also may be referred to as a C₅₋₂₀ aryloxy group). In some embodiments, ether refers to the group —OR, wherein R is a saturated C₁₋₇ alkyl group.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)ₙO(CR₂)ₙ—, repeating units of alkyloxy (e.g., polyethylenoxy, PEG, polymethyleneoxy) and alkylamino (e.g., polyethyleneamino, Jeffamine™); and diacid ester and amides including succinate, succinamide, diglycolate, malonate, and caproamide. In various embodiments, linkers can comprise one or more amino acid residues, such as valine, phenylalanine, lysine, and homolysine.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Leaving group” refers to a functional group that can be substituted by another functional group. Certain leaving groups are well known in the art, and examples include, but are not limited to, a halide (e.g., chloride, bromide, iodide), methanesulfonyl (mesyl), p-toluenesulfonyl (tosyl), trifluoromethylsulfonyl (triflate), and trifluoromethylsulfonate.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see T. W. Greene, Protective Groups in Organic Synthesis, John Wiley & Sons, New York, 1991, or a later edition.

### II. Compositions and Methods

In one aspect, the invention is based, in part, on antibodies that bind to LY6E and immunoconjugates comprising such antibodies. Antibodies and immunoconjugates of the invention are useful, e.g., for the diagnosis or treatment of LY6E-positive cancers.

### A. Exemplary Anti-Ly6E Antibodies

In some embodiments, the invention provides isolated antibodies that bind to LY6E. In certain embodiments, an anti-LY6E antibody has at least one or more of the following characteristics, in any combination.

A nonlimiting exemplary antibody of the invention is the Ly6E and humanized variants thereof. In some embodiments, Ly6E is human Ly6E, for example, human Ly6E of SEQ ID NO: 1. In some embodiments, Ly6E is selected from human, cynomolgus monkey, rhesus monkey, mouse or rat Ly6E.

In some such embodiments, the anti-Ly6E antibody binds Ly6E with an affinity of ≤25 nM, or ≤20 nM, or ≤15 nM, or ≤10 nM, or ≤9 nM, or ≤8 nM, or ≤7 nM, or ≤6 nM, or ≤5 nM, or ≤4 nM, or ≤3 nM, or ≤2 nM, or ≤1 nM, and optionally ≥0.0001 nM, or ≥0.001 nM, or ≥0.01 nM as measured by either surface plasma resonance (SPR) or scatchard analysis. In some such embodiments, the anti-Ly6E antibody binds Ly6E with an affinity of 0 to 25 nM, 0 to 20 nM, 0 to 10 nM, or 10 to 20 nM. In some embodiments, Ly6E is human Ly6E. In some embodiments, Ly6E is human Ly6E, mouse Ly6E, rat Ly6E, or cynomolgus monkey Ly6E. In some embodiments, the monovalent affinity for human Ly6E is 10 to 20 nM. In some embodiments, the bivalent affinity for human Ly6E is approximately three times more potent than monovalent affinity.

In one aspect, the invention provides an anti-Ly6E antibody comprising at least one, two, three, four, five, or six HVRs selected from (a) HVR-H1 comprising the amino acid sequence of SEQ ID NO: 10; (b) HVR-H2 comprising the amino acid sequence of SEQ ID NO:11; (c) HVR-H3 comprising the amino acid sequence of SEQ ID NO:12; (d) HVR-L1 comprising the amino acid sequence of SEQ ID NO:7; (e) HVR-L2 comprising the amino acid sequence of SEQ ID NO:8; and (f) HVR-L3 comprising the amino acid sequence of SEQ ID NO:9.

In one aspect, the invention provides an antibody comprising at least one, at least two, or all three VH HVR sequences selected from (a) HVR-H1 comprising the amino acid sequence of SEQ ID NO: 10; (b) HVR-H2 comprising the amino acid sequence of SEQ ID NO:11; and (c) HVR-H3 comprising the amino acid sequence of SEQ ID NO: 12. In one embodiment, the antibody comprises HVR-H3 comprising the amino acid sequence of SEQ ID NO:12. In another embodiment, the antibody comprises HVR-H3 comprising the amino acid sequence of SEQ ID NO: 12 and HVR-L3 comprising the amino acid sequence of SEQ ID NO:9. In a further embodiment, the antibody comprises HVR-H3 comprising the amino acid sequence of SEQ ID NO:12, HVR-L3 comprising the amino acid sequence of SEQ ID NO:9, and HVR-H2 comprising the amino acid sequence of SEQ ID NO: 11. In a further embodiment, the antibody comprises (a) HVR-H1 comprising the amino acid sequence of SEQ ID NO: 10; (b) HVR-H2 comprising the amino acid sequence of SEQ ID NO: 11; and (c) HVR-H3 comprising the amino acid sequence of SEQ ID NO: 12.

In another aspect, the invention provides an antibody comprising at least one, at least two, or all three VL HVR sequences selected from (a) HVR-L1 comprising the amino acid sequence of SEQ ID NO:7; (b) HVR-L2 comprising the amino acid sequence of SEQ ID NO:8; and (c) HVR-L3 comprising the amino acid sequence of SEQ ID NO:9. In one embodiment, the antibody comprises (a) HVR-L1 comprising the amino acid sequence of SEQ ID NO:7; (b) HVR-L2 comprising the amino acid sequence of SEQ ID NO:8; and (c) HVR-L3 comprising the amino acid sequence of SEQ ID NO:9.

In another aspect, an antibody of the invention comprises (a) a VH domain comprising at least one, at least two, or all three VH HVR sequences selected from (i) HVR-H1 comprising the amino acid sequence of SEQ ID NO: 10, (ii) HVR-H2 comprising the amino acid sequence of SEQ ID NO: 11, and (iii) HVR-H3 comprising an amino acid sequence selected from SEQ ID NO: 12; and (b) a VL domain comprising at least one, at least two, or all three VL HVR sequences selected from (i) HVR-L1 comprising the amino acid sequence of SEQ ID NO:7, (ii) HVR-L2 comprising the amino acid sequence of SEQ ID NO:8, and (c) HVR-L3 comprising the amino acid sequence of SEQ ID NO:9.

In another aspect, the invention provides an antibody comprising (a) HVR-H1 comprising the amino acid sequence of SEQ ID NO:10; (b) HVR-H2 comprising the amino acid sequence of SEQ ID NO: 11; (c) HVR-H3 comprising the amino acid sequence of SEQ ID NO: 12; (d) HVR-L1 comprising the amino acid sequence of SEQ ID NO:7; (e) HVR-L2 comprising the amino acid sequence of SEQ ID NO:8; and (f) HVR-L3 comprising an amino acid sequence selected from SEQ ID NO:9.

In any of the above embodiments, an anti-Ly6E antibody is humanized. In one embodiment, an anti-Ly6E antibody comprises HVRs as in any of the above embodiments, and further comprises an acceptor human framework, e.g., a human immunoglobulin framework or a human consensus framework.

In another aspect, an anti-Ly6E antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:32. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Ly6E antibody comprising that sequence retains the ability to bind to Ly6E. In certain embodiments, a total of 1 to 10 amino acids have been substituted, inserted and/or deleted in SEQ ID NO:32. In certain embodiments, substitutions, insertions, or deletions occur in regions outside the HVRs (i.e., in the FRs). Optionally, the anti-Ly6E antibody comprises the VH sequence in SEQ ID NO:32, including post-translational modifications of that sequence. In a particular embodiment, the VH comprises one, two or three HVRs selected from: (a) HVR-H1 comprising the amino acid sequence of SEQ ID NO: 10, (b) HVR-H2 comprising the amino acid sequence of SEQ ID NO: 11, and (c) HVR-H3 comprising the amino acid sequence of SEQ ID NO: 12.

In another aspect, an anti-Ly6E antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:31. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Ly6E antibody comprising that sequence retains the ability to bind to Ly6E. In certain embodiments, a total of 1 to 10 amino acids have been substituted, inserted and/or deleted in SEQ ID NO:31. In certain embodiments, the substitutions, insertions, or deletions occur in regions outside the HVRs (i.e., in the FRs). Optionally, the anti-Ly6E antibody comprises the VL sequence in SEQ ID NO:31, including post-translational modifications of that sequence. In a particular embodiment, the VL comprises one, two or three HVRs selected from (a) HVR-L1 comprising the amino acid sequence of SEQ ID NO:7; (b) HVR-L2 comprising the amino acid sequence of SEQ ID NO:8; and (c) HVR-L3 comprising the amino acid sequence of SEQ ID NO:9.

In another aspect, an anti-Ly6E antibody is provided, wherein the antibody comprises a VH as in any of the embodiments provided above, and a VL as in any of the embodiments provided above. In one embodiment, the antibody comprises the VH and VL sequences in SEQ ID NO:32 and SEQ ID NO:31, respectively, including post-translational modifications of those sequences.

In a further aspect, the invention provides an antibody that binds to the same epitope as an anti-Ly6E antibody provided herein. For example, in certain embodiments, an antibody is provided that binds to the same epitope as an anti-Ly6E antibody comprising a VH sequence of SEQ ID NO:32 and a VL sequence of SEQ ID NO:31.

In a further aspect, an anti-Ly6E antibody according to any of the above embodiments is a monoclonal antibody, including a chimeric, humanized or human antibody. In one embodiment, an anti-Ly6E antibody is an antibody fragment, e.g., a Fv, Fab, Fab′, scFv, diabody, or F(ab′)₂ fragment. In another embodiment, the antibody is a full-length antibody, e.g., an intact IgG1 antibody or other antibody class or isotype as defined herein.

In another aspect, an anti-Ly6E antibody is provided, wherein the antibody comprises an HC as in any of the embodiments provided above, and a VH as in any of the embodiments provided above. In some embodiments, the antibody comprises the HC in SEQ ID NO:5 and LC of SEQ ID NO:3, SEQ ID NO: 17, SEQ ID NO: 19, or SEQ ID NO: 29, including any post-translational modifications of those sequences. In one embodiment, the antibody comprises the HC in SEQ ID NO:5 and LC of SEQ ID NO:3, including any post-translational modifications of those sequences.

In a further aspect, an anti-Ly6E antibody according to any of the above embodiments may incorporate any of the features, singly or in combination, as described below.

### Assays

Whether an anti-Ly6E antibody “binds with an affinity of ≤25 nM, or ≤20 nM, or ≤15 nM, or ≤10 nM, or ≤9 nM, or ≤8 nM, or ≤7 nM, or ≤6 nM, or ≤5 nM, or ≤4 nM, or ≤3 nM, or ≤2 nM, or ≤1 nM,” or “0 to 25 nM, 0 to 20 nM, 0 to 10 nM, or 10 to 20 nM” is, in some embodiments, determined according to a scatchard analysis. Alternatively, an anti-Ly6E antibody affinity can be determined according to, for example, a BIAcore™ assay. Specifically, Kd is measured using surface plasmon resonance assays using a BIAcore™-3000 (BIAcore, Inc., Piscataway, NJ). BIAcore™ research grade CM5 chips are activated with 1-ethyl-3-(3-dimethylaminopropyl) carbodiimide (EDC) and N-hydroxysuccinimide (NHS) reagents according to the supplier's instructions. Goat anti-human Fc IgGs are coupled to the chips to achieve approximately 10,000 response units (RU) in each flow cell. Unreacted coupling groups are blocked with 1M ethanolamine. For kinetics measurements, anti-Ly6E antibodies are captured to achieve approximately 300 RU. Two-fold serial dilutions of human Ly6E are injected in HBS-P buffer (0.01M HEPES pH7.4, 0.15M NaCl, 0.005% surfactant P20) at 25° C. with a flow rate of 30 μl/min. Association rates (kₒₙ) and dissociation rates (k_{off}) are calculated using a 1:1 Langmuir binding model (BIAcore™ Evaluation Software version 3.2). The equilibrium dissociation constant (Kd) is calculated as the ratio k_{off}/kₒₙ. If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-Aminco® spectrophotometer (ThermoSpectronic) with a stirred cuvette.

In any of the above embodiments, an anti-Ly6E antibody is humanized. In one embodiment, an anti-Ly6E antibody comprises HVRs as in any of the above embodiments, and further comprises a human acceptor framework, e.g., a human immunoglobulin framework or a human consensus framework. In certain embodiments, the human acceptor framework is the human VL kappa IV consensus (VL_{KIV}) framework and/or the VH framework VH₁.

In a further aspect, an anti-Ly6E antibody according to any of the above embodiments is a monoclonal antibody, including a chimeric, humanized or human antibody. In one embodiment, an anti-Ly6E antibody is an antibody fragment, e.g., a Fv, Fab, Fab′, scFv, diabody, or F(ab′)₂ fragment. In another embodiment, the antibody is a substantially full length antibody, e.g., an IgG1 antibody or other antibody class or isotype as defined herein.

In a further aspect, an anti-Ly6E antibody according to any of the above embodiments may incorporate any of the features, singly or in combination, as described below.

In a further aspect, an anti-Ly6E antibody according to any of the above embodiments is a monoclonal antibody, including a human antibody. In one embodiment, an anti-Ly6E antibody is an antibody fragment, e.g., a Fv, Fab, Fab′, scFv, diabody, or F(ab′)₂ fragment. In another embodiment, the antibody is a substantially full length antibody, e.g., an IgG2a antibody or other antibody class or isotype as defined herein.

In a further aspect, an anti-Ly6E antibody according to any of the above embodiments may incorporate any of the features, singly or in combination, as described below.

### 1. Antibody Affinity

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM, and optionally is ≥10⁻¹³ M. (e.g., 10⁻⁸ M or less, e.g., from 10⁻⁸ M to 10⁻¹³ M, e.g., from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using scatchard analysis. According to another embodiment, Kd is measured using surface plasmon resonance assays using a BIAcore™-2000 or a BIAcore™-3000 (BIAcore, Inc., Piscataway, NJ) at 25° C. with immobilized antigen CM5 chips at ˜10 response units (RU). Briefly, carboxymethylated dextran biosensor chips (CM5, BIAcore, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIAcore™® Evaluation Software version 3.2) by simultaneously fitting the association and dissociation sensorgrams. The equilibrium dissociation constant (Kd) is calculated as the ratio k_{off}/kₒₙ. See, e.g., Chen et al., J Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall' Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., eds., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g., a bispecific antibody. Multispecific antibodies are monoclonal antibodies that have binding specificities for at least two different sites. In certain embodiments, one of the binding specificities is for LY6E and the other is for any other antigen. In certain embodiments, one of the binding specificities is for LY6E and the other is for CD3. See, e.g., U.S. Pat. No. 5,821,337. In certain embodiments, bispecific antibodies may bind to two different epitopes of Ly6E. Bispecific antibodies may also be used to localize cytotoxic agents to cells which express Ly6E. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to Ly6E as well as another, different antigen (see, e.g., US 2008/0069820).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of“exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al., Methods in Molecular Biology 178:1-37 (O'Brien et al., ed., Human Press, Totowa, NJ (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites are created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

Antibodies with increased half lives and improved binding to the neonatal Fc receptor (FcRn), which is responsible for the transfer of maternal IgGs to the fetus (Guyer et al., J Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an anti-LY6E antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an anti-LY6E antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-LY6E antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

### C. Assays

Anti-LY6E antibodies provided herein may be identified, screened for, or characterized for their physical/chemical properties and/or biological activities by various assays known in the art.

In one aspect, an antibody of the invention is tested for its antigen binding activity, e.g., by known methods such as ELISA, BIACore™, FACS, or Western blot.

In another aspect, competition assays may be used to identify an antibody that competes with any of the antibodies described herein for binding to LY6E. In certain embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized LY6E is incubated in a solution comprising a first labeled antibody that binds to LY6E (e.g., any of the antibodies described herein) and a second unlabeled antibody that is being tested for its ability to compete with the first antibody for binding to LY6E. The second antibody may be present in a hybridoma supernatant. As a control, immobilized LY6E is incubated in a solution comprising the first labeled antibody but not the second unlabeled antibody. After incubation under conditions permissive for binding of the first antibody to LY6E, excess unbound antibody is removed, and the amount of label associated with immobilized LY6E is measured. If the amount of label associated with immobilized LY6E is substantially reduced in the test sample relative to the control sample, then that indicates that the second antibody is competing with the first antibody for binding to LY6E. See Harlow and Lane (1988) Antibodies: A Laboratory Manual ch.14 (Cold Spring Harbor Laboratory, Cold Spring Harbor, NY).

### D. Immunoconjugates

The invention also provides immunoconjugates comprising an anti-LY6E antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radioconjugate).

Immunoconjugates allow for the targeted delivery of a drug moiety to a tumor, and, in some embodiments intracellular accumulation therein, where systemic administration of unconjugated drugs may result in unacceptable levels of toxicity to normal cells (Polakis P. (2005) Current Opinion in Pharmacology 5:382-387).

Antibody-drug conjugates (ADC) are targeted chemotherapeutic molecules which combine properties of both antibodies and cytotoxic drugs by targeting potent cytotoxic drugs to antigen-expressing tumor cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107.

The ADC compounds of the invention include those with anticancer activity. In some embodiments, the ADC compounds include an antibody conjugated, i.e., covalently attached, to the drug moiety. In some embodiments, the antibody is covalently attached to the drug moiety through a linker. The antibody-drug conjugates (ADC) of the invention selectively deliver an effective dose of a drug to tumor tissue whereby greater selectivity, i.e., a lower efficacious dose, may be achieved while increasing the therapeutic index (“therapeutic window”).

The drug moiety (D) of the antibody-drug conjugates (ADC) may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, pyrrolobenzodiazepine (PBD), nemorubicin and its derivatives, PNU-159682, anthracycline, duocarmycin, vinca alkaloid, taxane, trichothecene, CC1065, camptothecin, elinafide, and stereoisomers, isosteres, analogs, and derivatives thereof that have cytotoxic activity. Nonlimiting examples of such immunoconjugates are discussed in further detail below.

### 1. Exemplary Antibody-drug Conjugates

An exemplary embodiment of an antibody-drug conjugate (ADC) compound comprises an antibody (Ab) which targets a tumor cell, a drug moiety (D), and a linker moiety (L) that attaches Ab to D. In some embodiments, the antibody is attached to the linker moiety (L) through one or more amino acid residues, such as lysine and/or cysteine.

An exemplary ADC has Formula I:

Ab-(L-D)ₚ Formula I

where p is 1 to about 20. In some embodiments, the number of drug moieties that can be conjugated to an antibody is limited by the number of free cysteine residues. In some embodiments, free cysteine residues are introduced into the antibody amino acid sequence by the methods described herein. Exemplary ADC of Formula I include, but are not limited to, antibodies that have 1, 2, 3, or 4 engineered cysteine amino acids (Lyon, R. et al (2012) Methods in Enzym. 502:123-138). In some embodiments, one or more free cysteine residues are already present in an antibody, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

### a) Exemplary Linkers

A “Linker” (L) is a bifunctional or multifunctional moiety that can be used to link one or more drug moieties (D) to an antibody (Ab) to form an antibody-drug conjugate (ADC) of Formula I. In some embodiments, antibody-drug conjugates (ADC) can be prepared using a Linker having reactive functionalities for covalently attaching to the drug and to the antibody. For example, in some embodiments, a cysteine thiol of an antibody (Ab) can form a bond with a reactive functional group of a linker or a drug-linker intermediate to make an ADC.

In one aspect, a linker has a functionality that is capable of reacting with a free cysteine present on an antibody to form a covalent bond. Nonlimiting exemplary such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on an antibody. Exemplary such electrophilic groups include, but are not limited to, aldehyde and ketone carbonyl groups. In some embodiments, a heteroatom of the reactive functionality of the linker can react with an electrophilic group on an antibody and form a covalent bond to an antibody unit. Nonlimiting exemplary such reactive functionalities include, but are not limited to, hydrazide, oxime, amino, hydrazine, thiosemicarbazone, hydrazine carboxylate, and arylhydrazide.

A linker may comprise one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl) cyclohexane-1 carboxylate (“MCC”). Various linker components are known in the art, some of which are described below.

A linker may be a “cleavable linker,” facilitating release of a drug. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker has the following Formula II:

-Aₐ-W_{w}—Y_{y}— Formula II
wherein A is a “stretcher unit”, and a is an integer from 0 to 1; W is an “amino acid unit”, and w is an integer from 0 to 12; Y is a “spacer unit”, and y is 0, 1, or 2; and Ab, D, and p are defined as above for Formula I. Exemplary embodiments of such linkers are described in U.S. Pat. No. 7,498,298, which is expressly incorporated herein by reference.

In some embodiments, a linker component comprises a “stretcher unit” that links an antibody to another linker component or to a drug moiety. Nonlimiting exemplary stretcher units are shown below (wherein the wavy line indicates sites of covalent attachment to an antibody, drug, or additional linker components):

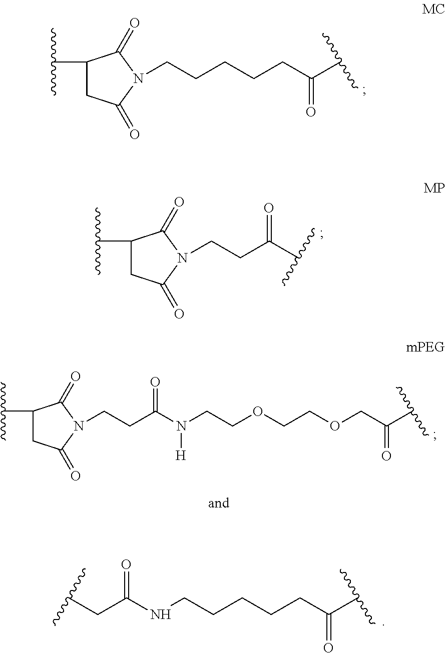 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component comprises an “amino acid unit”. In some such embodiments, the amino acid unit allows for cleavage of the linker by a protease, thereby facilitating release of the drug from the immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

In some embodiments, a linker component comprises a “spacer” unit that links the antibody to a drug moiety, either directly or through a stretcher unit and/or an amino acid unit. A spacer unit may be “self-immolative” or a “non-self-immolative.” A “non-self-immolative” spacer unit is one in which part or all of the spacer unit remains bound to the drug moiety upon cleavage of the ADC. Examples of non-self-immolative spacer units include, but are not limited to, a glycine spacer unit and a glycine-glycine spacer unit. In some embodiments, enzymatic cleavage of an ADC containing a glycine-glycine spacer unit by a tumor-cell associated protease results in release of a glycine-glycine-drug moiety from the remainder of the ADC. In some such embodiments, the glycine-glycine-drug moiety is subjected to a hydrolysis step in the tumor cell, thus cleaving the glycine-glycine spacer unit from the drug moiety.

A “self-immolative” spacer unit allows for release of the drug moiety. In certain embodiments, a spacer unit of a linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the spacer unit is p-aminobenzyloxycarbonyl (PAB). In some embodiments, an ADC comprising a self-immolative linker has the structure:

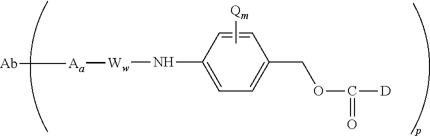

wherein Q is —C₁-C₈ alkyl, —O—(C₁-C₈ alkyl), -halogen, -nitro, or -cyano; m is an integer ranging from 0 to 4; and p ranges from 1 to about 20. In some embodiments, p ranges from 1 to 10, 1 to 7, 1 to 5, or 1 to 4.

Other examples of self-immolative spacers include, but are not limited to, aromatic compounds that are electronically similar to the PAB group, such as 2-aminoimidazol-5-methanol derivatives (U.S. Pat. No. 7,375,078; Hay et al., (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al., (1995) Chemistry Biology 2:223), appropriately substituted bicyclic[2.2.1] and bicyclic[2.2.2] ring systems (Storm et al., (1972) J. Amer. Chem. Soc. 94:5815) and 2-aminophenylpropionic acid amides (Amsberry et al., (1990) J. Org. Chem. 55:5867). Linkage of a drug to the α-carbon of a glycine residue is another example of a self-immolative spacer that may be useful in ADC (Kingsbury et al., (1984) J. Med. Chem. 27:1447).

In some embodiments, linker L may be a dendritic type linker for covalent attachment of more than one drug moiety to an antibody through a branching, multifunctional linker moiety (Sun et al., (2002) Bioorganic & Medicinal Chemistry Letters 12:2213-2215; Sun et al., (2003) Bioorganic & Medicinal Chemistry 11:1761-1768). Dendritic linkers can increase the molar ratio of drug to antibody, i.e., loading, which is related to the potency of the ADC. Thus, where an antibody bears only one reactive cysteine thiol group, a multitude of drug moieties may be attached through a dendritic linker.

Nonlimiting exemplary linkers are shown below in the context of an ADC of Formula I:

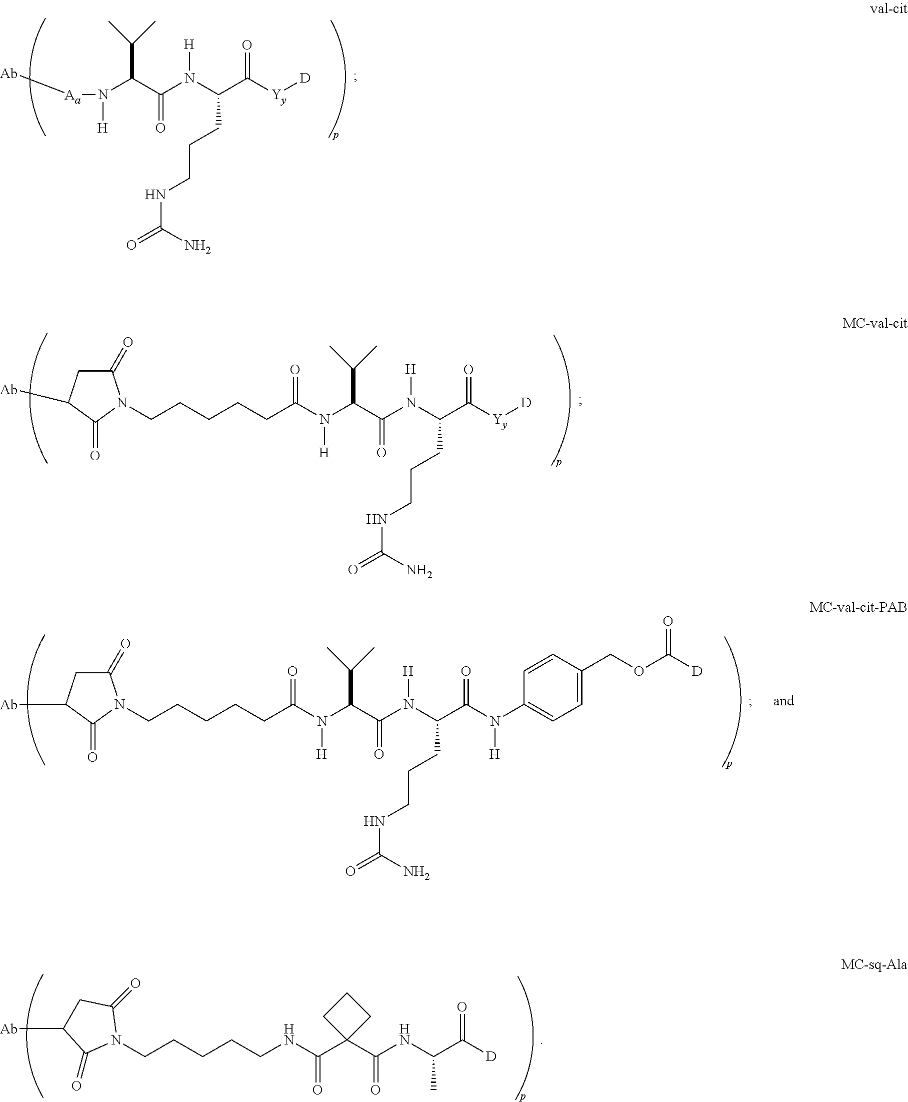

Further nonlimiting exemplary ADCs include the following structures where —S— is part of the antibody:

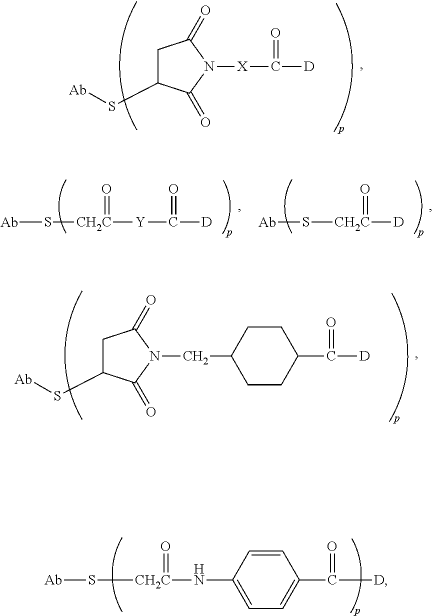

where X is:

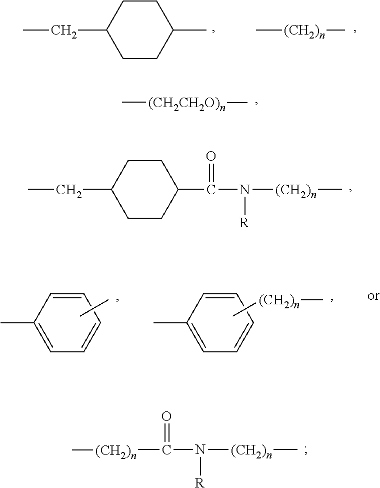

Y is:

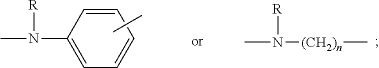

each R is independently H or C₁-C₆ alkyl; and n is 1 to 12.

Typically, peptide-type linkers can be prepared by forming a peptide bond between two or more amino acids and/or peptide fragments. Such peptide bonds can be prepared, for example, according to a liquid phase synthesis method (e.g., E. Schröder and K. Lübke (1965) “The Peptides”, volume 1, pp 76-136, Academic Press).

In some embodiments, a linker is substituted with groups that modulate solubility and/or reactivity. As a nonlimiting example, a charged substituent such as sulfonate (—SO₃⁻) or ammonium may increase water solubility of the linker reagent and facilitate the coupling reaction of the linker reagent with the antibody and/or the drug moiety, or facilitate the coupling reaction of Ab-L (antibody-linker intermediate) with D, or D-L (drug-linker intermediate) with Ab, depending on the synthetic route employed to prepare the ADC. In some embodiments, a portion of the linker is coupled to the antibody and a portion of the linker is coupled to the drug, and then the Ab-(linker portion)^{a} is coupled to drug-(linker portion)^{b} to form the ADC of Formula I. In some such embodiments, the antibody comprises more than one (linker portion)^{a} substituents, such that more than one drug is coupled to the antibody in the ADC of Formula I.

The compounds of the invention expressly contemplate, but are not limited to, ADC prepared with the following linker reagents: bis-maleimido-trioxyethylene glycol (BMPEO), N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(beta-maleimidopropionamido)hexanoate](SMPH), iminothiolane (IT), sulfo-EMCS, sulfo-GMBS, sulfo-KMUS, sulfo-MBS, sulfo-SIAB, sulfo-SMCC, and sulfo-SMPB, and succinimidyl-(4-vinylsulfone)benzoate (SVSB), and including bis-maleimide reagents: dithiobismaleimidoethane (DTME), 1,4-Bismaleimidobutane (BMB), 1,4 Bismaleimidyl-2,3-dihydroxybutane (BMDB), bismaleimidohexane (BMH), bismaleimidoethane (BMOE), BM(PEG)₂ (shown below), and BM(PEG)₃ (shown below); bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In some embodiments, bis-maleimide reagents allow the attachment of the thiol group of a cysteine in the antibody to a thiol-containing drug moiety, linker, or linker-drug intermediate. Other functional groups that are reactive with thiol groups include, but are not limited to, iodoacetamide, bromoacetamide, vinyl pyridine, disulfide, pyridyl disulfide, isocyanate, and isothiocyanate.

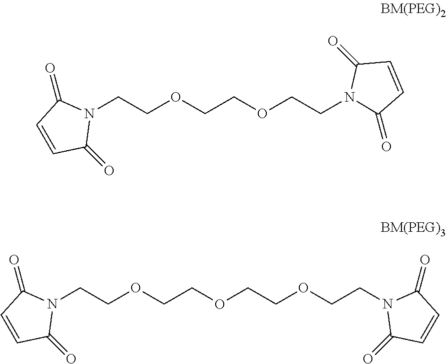 [SMILES: O=C1C=CC(=O)N1CCOCCOCCN1C(=O)C=CC1=O ; O=C1C=CC(=O)N1CCOCCOCCOCCN1C(=O)C=CC1=O]

In some embodiments, the linker is an MC-sq-Ala linker. Examples of peptidomimetic linkers are available, for example, in WO2015/095227 A2.

Certain useful linker reagents can be obtained from various commercial sources, such as Pierce Biotechnology, Inc. (Rockford, IL), Molecular Biosciences Inc. (Boulder, CO), or synthesized in accordance with procedures described in the art; for example, in Toki et al., (2002) J. Org. Chem. 67:1866-1872; Dubowchik et al., (1997) Tetrahedron Letters, 38:5257-60; Walker, M. A. (1995) J. Org. Chem. 60:5352-5355; Frisch et al., (1996) Bioconjugate Chem. 7:180-186; U.S. Pat. No. 6,214,345; WO 02/088172; US 2003130189; US2003096743; WO 03/026577; WO 03/043583; and WO 04/032828.

Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026.

### b) Exemplary Drug Moieties

### Pyrrolobenzodiazepines

In some embodiments, an ADC comprises a pyrrolobenzodiazepine (PBD). In some embodiments, PDB dimers recognize and bind to specific DNA sequences. The natural product anthramycin, a PBD, was first reported in 1965 (Leimgruber et al., (1965) J. Am. Chem. Soc., 87:5793-5795; Leimgruber et al., (1965) J Am. Chem. Soc., 87:5791-5793). Since then, a number of PBDs, both naturally-occurring and analogues, have been reported (Thurston et al., (1994) Chem. Rev. 1994, 433-465 including dimers of the tricyclic PBD scaffold (U.S. Pat. Nos. 6,884,799; 7,049,311; 7,067,511; 7,265,105; 7,511,032; 7,528,126; 7,557,099). Without intending to be bound by any particular theory, it is believed that the dimer structure imparts the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, (1986) Acc. Chem. Res., 19:230-237). Dimeric PBD compounds bearing C2 aryl substituents have been shown to be useful as cytotoxic agents (Hartley et al., (2010) Cancer Res. 70(17):6849-6858; Antonow (2010) J. Med. Chem. 53(7):2927-2941; Howard et al., (2009) Bioorganic and Med. Chem. Letters 19(22):6463-6466).

In some embodiments, PBD compounds can be employed as prodrugs by protecting them at the N10 position with a nitrogen protecting group which is removable in vivo (WO 00/12507; WO 2005/023814).

PBD dimers have been conjugated to antibodies and the resulting ADC shown to have anti-cancer properties (US 2010/0203007). Nonlimiting exemplary linkage sites on the PBD dimer include the five-membered pyrrolo ring, the tether between the PBD units, and the N10-C11 imine group (WO 2009/016516; US 2009/304710; US 2010/047257; US 2009/036431; US 2011/0256157; WO 2011/130598).

In some embodiments of the ADCs provided herein, the drug moiety (for example, drug D) is a compound of Formula A:

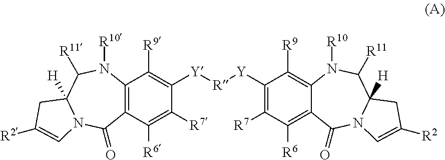

wherein:
R² is of formula II:

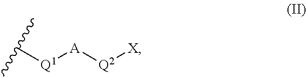

wherein A is a C_{5_7} aryl group, X is selected from the group consisting of: OH, SH, CO₂H, COH, N═C═O, NHR^{N}, and (OC₂H₄)ₘOCH₃, wherein R^{N} is selected from the group consisting of H and C₁₋₄ alkyl, and wherein m is an integer from 1 to 3, and either:
(i) Q¹ is a single bond, and Q² is selected from a single bond and —Z—(CH₂)ₙ—, wherein Z is a single bond, O, S, or NH and n is an integer from 1 to 3; or
(ii) Q¹ is —CH═CH—, and Q² is a single bond;
R^{2′} is a C_{5_10} aryl group, optionally substituted by one or more substituents selected from the group consisting of halo, nitro, cyano, ether, C₁₋₇ alkyl, C₃₋₇ heterocyclyl, and bis-oxy-C₁₋₃ alkylene;
R⁶ and R⁹ are independently selected from H, R, OH, OR, SH, SR, NH₂, NHR, NRR′, nitro,
Me₃Sn and halo;
R⁷ is selected from the group consisting of H, R, OH, OR, SH, SR, NH₂, NHR, NHRR′, nitro, Me₃Sn, and halo;
R and R′ are independently selected from optionally substituted C₁₋₁₂ alkyl, C₃₋₂₀ heterocyclyl and C₅₋₂₀ aryl groups;
either:
   (a) R¹⁰ is H, and R¹¹ is OH or OR^{A}, wherein R^{A} is C₁₋₄ alkyl;
   (b) R¹⁰ and R¹¹ form a nitrogen-carbon double bond between the nitrogen and carbon atoms to which they are bound; or
   (c) R¹⁰ is H and R¹¹ is SO_{z}M, wherein z is 2 or 3 and M is a monovalent pharmaceutically acceptable cation;
R″ is a C₃₋₁₂ alkylene group, which chain may be interrupted by one or more heteroatoms independently selected from the group consisting of O, S, and NH, and/or one or more aromatic rings independently selected from the group consisting of benzene or pyridine;
Y is O, S, or NH;
R^{6′}, R^{7′}, and R^{9′} are selected from the same groups as R⁶, R⁷ and R⁹ respectively, and R^{10′} and R^{11′} are the same as R¹⁰ and R¹¹ respectively, wherein if R¹¹ and R^{11′} are SO_{z}M, M may represent a divalent pharmaceutically acceptable cation; and
the point of attachment to the linker L is through R² or R^{2′}.

In some embodiments of Formula (A):
R² is of formula II:

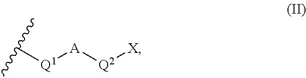

wherein A is phenyl, X is selected from the group consisting of: OH, SH, CO₂H, COH, N═C═O, NHR^{N}, and (OC₂H₄)ₘOCH₃, wherein R^{N} is selected from the group consisting of H and C₁₋₄ alkyl, and wherein m is an integer from 1 to 3, and:
(i) Q¹ is a single bond, and Q² is a single bond;
R^{2′} is a phenyl group, optionally substituted by one or more substituents selected from the group consisting of halo, nitro, cyano, and —OR, wherein R is a saturated C₁₋₇ alkyl group.;
R⁶ and R⁹ are independently selected from H, R, OH, OR, SH, SR, NH₂, NHR, NRR′, nitro, Me₃Sn and halo;
R⁷ is selected from the group consisting of H, R, OH, OR, SH, SR, NH₂, NHR, NRR′, nitro, Me₃Sn, and halo;
R and R′ are independently selected from unsubstituted C₁₋₁₂ saturated alkyl;
R¹⁰ and R¹¹ form a nitrogen-carbon double bond between the nitrogen and carbon atoms to which they are bound;
R″ is a C₃₋₁₂ saturated alkylene group, which chain may be interrupted by one or two heteroatoms independently selected from the group consisting of O, S, and NH;
Y is O, S, or NH;
R^{6′}, R^{7′}, and R^{9′} are selected from the same groups as R⁶, R⁷ and R⁹ respectively, and R^{10′} and R^{11′} are the same as R¹⁰ and R¹¹ respectively; and
the point of attachment to the linker L is through R² or R^{2′}.

Exemplary PDB portions of an ADC include, but are not limited to (wherein the wavy line indicates the site of covalent attachment to the linker):

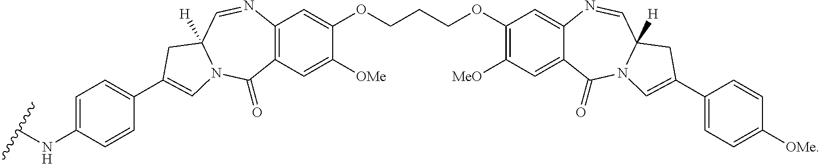 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

A nonlimiting exemplary linker-PBD has the following structure:

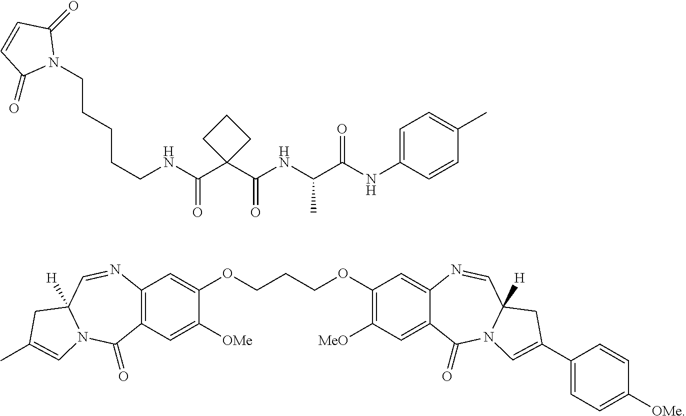

A nonlimiting exemplary linker-PBD portion of an ADC includes, but is not limited to:

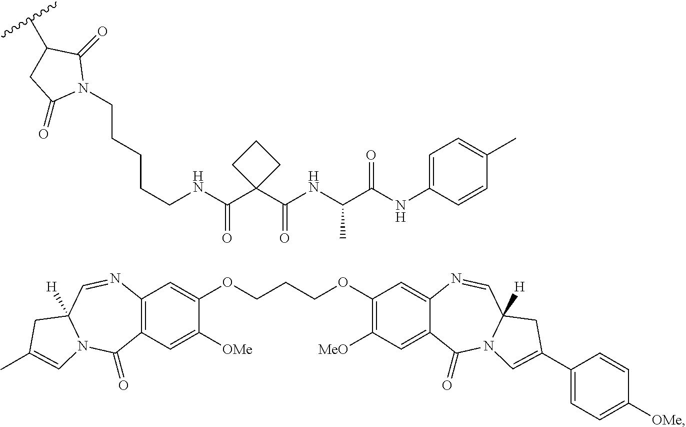

wherein the wavy line indicates the site of covalent attachment to the antibody.

PBDs and ADC comprising PBDs may be prepared according to methods known in the art. See, e.g., WO 2009/016516; US 2009/304710; US 2010/047257; US 2009/036431; US 2011/0256157; WO 2011/130598; and WO2010/043880.

### c) Drug Loading

Drug loading is represented by p, the average number of drug moieties per antibody in a molecule of Formula I. Drug loading may range from 1 to 20 drug moieties (D) per antibody. ADCs of Formula I include collections of antibodies conjugated with a range of drug moieties, from 1 to 20. The average number of drug moieties per antibody in preparations of ADC from conjugation reactions may be characterized by conventional means such as mass spectroscopy, ELISA assay, and HPLC. The quantitative distribution of ADC in terms of p may also be determined. In some instances, separation, purification, and characterization of homogeneous ADC where p is a certain value from ADC with other drug loadings may be achieved by means such as reverse phase HPLC or electrophoresis.

For some antibody-drug conjugates, p may be limited by the number of attachment sites on the antibody. For example, where the attachment is a cysteine thiol, as in certain exemplary embodiments above, an antibody may have only one or several cysteine thiol groups, or may have only one or several sufficiently reactive thiol groups through which a linker may be attached. In certain embodiments, higher drug loading, e.g., p>5, may cause aggregation, insolubility, toxicity, or loss of cellular permeability of certain antibody-drug conjugates. In certain embodiments, the average drug loading for an ADC ranges from 1 to about 8; from about 2 to about 6; or from about 3 to about 5. Indeed, it has been shown that for certain ADCs, the optimal ratio of drug moieties per antibody may be less than 8, and may be about 2 to about 5 (U.S. Pat. No. 7,498,298).

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio) of an ADC may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g., hydrophobic interaction chromatography (see, e.g., McDonagh et al., (2006) Prot. Engr. Design & Selection 19(7):299-307; Hamblett et al., (2004) Clin. Cancer Res. 10:7063-7070; Hamblett et al., “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley et al., “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value may be isolated from the conjugation mixture by electrophoresis or chromatography.

### d) Certain Methods of Preparing Immunoconjugates

An ADC of Formula I may be prepared by several routes employing organic chemistry reactions, conditions, and reagents known to those skilled in the art, including: (1) reaction of a nucleophilic group of an antibody with a bivalent linker reagent to form Ab-L via a covalent bond, followed by reaction with a drug moiety D; and (2) reaction of a nucleophilic group of a drug moiety with a bivalent linker reagent, to form D-L, via a covalent bond, followed by reaction with a nucleophilic group of an antibody. Exemplary methods for preparing an ADC of Formula I via the latter route are described in U.S. Pat. No. 7,498,298, which is expressly incorporated herein by reference.

Nucleophilic groups on antibodies include, but are not limited to: (i) N-terminal amine groups, (ii) side chain amine groups, e.g., lysine, (iii) side chain thiol groups, e.g., cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, i.e., cysteine bridges. Antibodies may be made reactive for conjugation with linker reagents by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into antibodies through modification of lysine residues, e.g., by reacting lysine residues with 2-iminothiolane (Traut's reagent), resulting in conversion of an amine into a thiol. Reactive thiol groups may also be introduced into an antibody by introducing one, two, three, four, or more cysteine residues (e.g., by preparing variant antibodies comprising one or more non-native cysteine amino acid residues).

Antibody-drug conjugates of the invention may also be produced by reaction between an electrophilic group on an antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an antibody is modified to introduce electrophilic moieties that are capable of reacting with nucleophilic substituents on the linker reagent or drug. In another embodiment, the sugars of glycosylated antibodies may be oxidized, e.g., with periodate oxidizing reagents, to form aldehyde or ketone groups which may react with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody that can react with appropriate groups on the drug (Hermanson, Bioconjugate Techniques). In another embodiment, antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Exemplary nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare ADC are described herein in the section titled “Exemplary Linkers.” Methods of using such cross-linker reagents to link two moieties, including a proteinaceous moiety and a chemical moiety, are known in the art. In some embodiments, a fusion protein comprising an antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate.

In yet another embodiment, an antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a drug or radionucleotide).

### E. Methods and Compositions for Diagnostics and Detection

In certain embodiments, any of the anti-LY6E antibodies provided herein are useful for detecting the presence of LY6E in a biological sample. The term “detecting” as used herein encompasses quantitative or qualitative detection. A “biological sample” comprises, e.g., a cell or tissue (e.g., biopsy material, including cancerous or potentially cancerous colon, colorectal, endometrial, pancreatic, or ovarian tissue).

In one embodiment, an anti-LY6E antibody for use in a method of diagnosis or detection is provided. In a further aspect, a method of detecting the presence of LY6E in a biological sample is provided. In certain embodiments, the method comprises contacting the biological sample with an anti-LY6E antibody as described herein under conditions permissive for binding of the anti-LY6E antibody to LY6E, and detecting whether a complex is formed between the anti-LY6E antibody and LY6E in the biological sample. Such method may be an in vitro or in vivo method. In one embodiment, an anti-LY6E antibody is used to select subjects eligible for therapy with an anti-LY6E antibody, e.g., where LY6E is a biomarker for selection of patients. In a further embodiment, the biological sample is a cell or tissue (e.g., biopsy material, including cancerous or potentially cancerous colon, colorectal, endometrial, pancreatic, or ovarian tissue).

In a further embodiment, an anti-LY6E antibody is used in vivo to detect, e.g., by in vivo imaging, A LY6E-positive cancer in a subject, e.g., for the purposes of diagnosing, prognosing, or staging cancer, determining the appropriate course of therapy, or monitoring response of a cancer to therapy. One method known in the art for in vivo detection is immuno-positron emission tomography (immuno-PET), as described, e.g., in van Dongen et al., The Oncologist 12:1379-1389 (2007) and Verel et al., J. Nucl. Med. 44:1271-1281 (2003). In such embodiments, a method is provided for detecting A LY6E-positive cancer in a subject, the method comprising administering a labeled anti-LY6E antibody to a subject having or suspected of having A LY6E-positive cancer, and detecting the labeled anti-LY6E antibody in the subject, wherein detection of the labeled anti-LY6E antibody indicates A LY6E-positive cancer in the subject. In certain of such embodiments, the labeled anti-LY6E antibody comprises an anti-LY6E antibody conjugated to a positron emitter, such as ⁶⁸Ga, ¹⁸F, ⁶⁴Cu, ⁸⁶Y, ⁷⁶Br, ⁸⁹Zr, and ¹²⁴I. In a particular embodiment, the positron emitter is ⁸⁹Zr.

In further embodiments, a method of diagnosis or detection comprises contacting a first anti-LY6E antibody immobilized to a substrate with a biological sample to be tested for the presence of LY6E, exposing the substrate to a second anti-LY6E antibody, and detecting whether the second anti-LY6E is bound to a complex between the first anti-LY6E antibody and LY6E in the biological sample. A substrate may be any supportive medium, e.g., glass, metal, ceramic, polymeric beads, slides, chips, and other substrates. In certain embodiments, a biological sample comprises a cell or tissue (e.g., biopsy material, including cancerous or potentially cancerous colorectal, endometrial, pancreatic or ovarian tissue). In certain embodiments, the first or second anti-LY6E antibody is any of the antibodies described herein. In such embodiments, the second anti-LY6E antibody may be 6D3 or 7C9; or antibodies derived from 6D3 or 7C9 as described herein.

Exemplary disorders that may be diagnosed or detected according to any of the above embodiments include LY6E-positive cancers, such as LY6E-positive colorectal cancer (including adenocarcinoma), LY6E-positive ovarian cancer (including ovarian serous adenocarcinoma), LY6E-positive pancreatic cancer (including pancreatic ductal adenocarcinoma), and LY6E-positive endometrial cancer. In some embodiments, A LY6E-positive cancer is a cancer that receives an anti-LY6E immunohistochemistry (IHC) or in situ hybridization (ISH) score greater than “0,” which corresponds to very weak or no staining in >90% of tumor cells. In another embodiment, A LY6E-positive cancer expresses LY6E at a 1+, 2+ or 3+ level. In some embodiments, A LY6E-positive cancer is a cancer that expresses LY6E according to a reverse-transcriptase PCR (RT-PCR) assay that detects LY6E mRNA. In some embodiments, the RT-PCR is quantitative RT-PCR.

In certain embodiments, labeled anti-LY6E antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, 0-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. In another embodiment, a label is a positron emitter. Positron emitters include but are not limited to ⁶⁸Ga, ¹⁸F, ⁶⁴Cu, ⁸⁶Y, ⁷⁶Br, ⁸⁹Zr, and ¹²⁴I. In a particular embodiment, a positron emitter is ⁸⁹Zr.

### F. Pharmaceutical Formulations

Pharmaceutical formulations of an anti-LY6E antibody or immunoconjugate as described herein are prepared by mixing such antibody or immunoconjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, in some instances, it may be desirable to further provide a platinum complex, e.g., for the treatment of LY6E-positive cancer such as, for example, a LY6E-positive breast cancer, or a LY6E-positive pancreatic cancer, or a LY6E-positive colon cancer, or a LY6E-positive colorectal cancer, or a LY6E-positive melanoma cancer, or a LY6E-positive ovarian cancer, or a LY6E-positive non-small cell lung cancer, or a LY6E-positive gastric cancer.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g., films, or microcapsules.

The formulations to be used for in vivo administration are generally sterile. Sterility may be readily accomplished, e.g., by filtration through sterile filtration membranes.

### G. Therapeutic Methods and Compositions

Any of the anti-LY6E antibodies or immunoconjugates provided herein may be used in methods, e.g., therapeutic methods.

In one aspect, an anti-LY6E antibody or immunoconjugate provided herein is used in a method of inhibiting proliferation of a LY6E-positive cell, the method comprising exposing the cell to the anti-LY6E antibody or immunoconjugate under conditions permissive for binding of the anti-LY6E antibody or immunoconjugate to LY6E on the surface of the cell, thereby inhibiting the proliferation of the cell. In certain embodiments, the method is an in vitro or an in vivo method. In further embodiments, the cell is a breast cancer cell or a pancreatic cancer cell or a colon cancer cell, or a colorectal cancer cell, or a melanoma cancer cell, or an ovarian cancer cell, or a non-small cell lung cancer cell, or a gastric cancer cell.

Inhibition of cell proliferation in vitro may be assayed using the CellTiter-Glo™ Luminescent Cell Viability Assay, which is commercially available from Promega (Madison, WI). That assay determines the number of viable cells in culture based on quantitation of ATP present, which is an indication of metabolically active cells. See Crouch et al., (1993) J. Immunol. Meth. 160:81-88, U.S. Pat. No. 6,602,677. The assay may be conducted in 96- or 384-well format, making it amenable to automated high-throughput screening (HTS). See Cree et al., (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

In another aspect, an anti-LY6E antibody or immunoconjugate for use as a medicament is provided. In further aspects, an anti-LY6E antibody or immunoconjugate for use in a method of treatment is provided. In certain embodiments, an anti-LY6E antibody or immunoconjugate for use in treating LY6E-positive cancer is provided. In certain embodiments, the invention provides an anti-LY6E antibody or immunoconjugate for use in a method of treating an individual having a LY6E-positive cancer, the method comprising administering to the individual an effective amount of the anti-LY6E antibody or immunoconjugate. In one such embodiment, the method further comprises administering to the individual an effective amount of at least one additional therapeutic agent, e.g., as described below.

In a further aspect, the invention provides for the use of an anti-LY6E antibody or immunoconjugate in the manufacture or preparation of a medicament. In one embodiment, the medicament is for treatment of LY6E-positive cancer. In a further embodiment, the medicament is for use in a method of treating LY6E-positive cancer, the method comprising administering to an individual having LY6E-positive cancer an effective amount of the medicament. In one such embodiment, the method further comprises administering to the individual an effective amount of at least one additional therapeutic agent, e.g., as described below.

In a further aspect, the invention provides a method for treating LY6E-positive cancer. In one embodiment, the method comprises administering to an individual having such LY6E-positive cancer an effective amount of an anti-LY6E antibody or immunoconjugate. In one such embodiment, the method further comprises administering to the individual an effective amount of at least one additional therapeutic agent, as described below.

A LY6E-positive cancer according to any of the above embodiments may be, e.g., LY6E-positive breast cancer, or LY6E-positive pancreatic cancer, or LY6E-positive colon cancer, or LY6E-positive colorectal cancer, or LY6E-positive melanoma cancer, or LY6E-positive ovarian cancer, or LY6E-positive non-small cell lung cancer, or LY6E-positive gastric cancer. In some embodiments, a LY6E-positive cancer is a cancer that receives an anti-LY6E immunohistochemistry (IHC) or in situ hybridization (ISH) score greater than “0,” which corresponds to very weak or no staining in >90% of tumor cells, under the conditions described herein. In another embodiment, a LY6E-positive cancer expresses LY6E at a 1+, 2+ or 3+ level, as defined under the conditions described herein. In some embodiments, a LY6E-positive cancer is a cancer that expresses LY6E according to a reverse-transcriptase PCR (RT-PCR) assay that detects LY6E mRNA. In some embodiments, the RT-PCR is quantitative RT-PCR.

An “individual” according to any of the above embodiments may be a human.

In a further aspect, the invention provides pharmaceutical formulations comprising any of the anti-LY6E antibodies or immunoconjugate provided herein, e.g., for use in any of the above therapeutic methods. In one embodiment, a pharmaceutical formulation comprises any of the anti-LY6E antibodies or immunoconjugates provided herein and a pharmaceutically acceptable carrier. In another embodiment, a pharmaceutical formulation comprises any of the anti-LY6E antibodies or immunoconjugates provided herein and at least one additional therapeutic agent, e.g., as described below.

Antibodies or immunoconjugates of the invention can be used either alone or in combination with other agents in a therapy. For instance, an antibody or immunoconjugate of the invention may be co-administered with at least one additional therapeutic agent. In certain embodiments, an additional therapeutic agent is a platinum complex, e.g., for the treatment of LY6E-positive cancer such as, for example, a LY6E-positive breast cancer, or a LY6E-positive pancreatic cancer, or a LY6E-positive colon cancer, or a LY6E-positive colorectal cancer, or a LY6E-positive melanoma cancer, or a LY6E-positive ovarian cancer, or a LY6E-positive non-small cell lung cancer, or a LY6E-positive gastric cancer.

Such combination therapies noted above encompass combined administration (where two or more therapeutic agents are included in the same or separate formulations), and separate administration, in which case, administration of the antibody or immunoconjugate of the invention can occur prior to, simultaneously, and/or following, administration of the additional therapeutic agent and/or adjuvant. Antibodies or immunoconjugates of the invention can also be used in combination with radiation therapy.

An antibody or immunoconjugate of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or immunoconjugates of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody or immunoconjugate need not be, but is optionally formulated with one or more agents currently used to prevent or treat the disorder in question. The effective amount of such other agents depends on the amount of antibody or immunoconjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or immunoconjugate of the invention (when used alone or in combination with one or more other additional therapeutic agents) will depend on the type of disease to be treated, the type of antibody or immunoconjugate, the severity and course of the disease, whether the antibody or immunoconjugate is administered for preventive or therapeutic purposes, previous therapy, the patient's clinical history and response to the antibody or immunoconjugate, and the discretion of the attending physician. The antibody or immunoconjugate is suitably administered to the patient at one time or over a series of treatments. Depending on the type and severity of the disease, about 1 μg/kg to 15 mg/kg (e.g., 0.1 mg/kg-10 mg/kg) of antibody or immunoconjugate can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody or immunoconjugate would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using both an immunoconjugate of the invention and an anti-LY6E antibody.

### H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disorder and may have a sterile access port (for example the container may be an intravenous solution bag or a vial having a stopper pierceable by a hypodermic injection needle). At least one active agent in the composition is an antibody or immunoconjugate of the invention. The label or package insert indicates that the composition is used for treating the condition of choice. Moreover, the article of manufacture may comprise (a) a first container with a composition contained therein, wherein the composition comprises an antibody or immunoconjugate of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

### I. Sequences

In another aspect, the following sequences useful for the treatment, prevention and/or diagnosis of the disorders described herein are provided.

| SEQ ID NO: | SEQUENCE | DESCRIPTION |
|---|---|---|
| 1 | MKIFLPVLLAALLGVERASSLMCFSCLNQKSN | HUMAN Ly6E amino acid |
| | LYCLKPTICSDQDNYCVTVSASAGIGNLVTFG | sequence with signal sequence |
| | HSLSKTCSPACPIPEGVNVGVASMGISCCQSF | (amino acids 1-20 underlined) |
| | LCNFSAADGGLRASVTLLGAGLLLSLLPALLR | |
| | FGP | |
| 2 | MKIFLPVLLAALLGVERASSLMCFSCLNQKSN | CYNOMOLOGOUS Ly6E |
| | LYCLKPTICSDQDNYCVTVSTSAGIGNLVTFG | amino acid sequence with |
| | HSLSKTCSPACPLPEGINVGVASMGISCCQSF | signal sequence (amino acids |
| | LCNFSAADGGLRASATLLGAGLLLSLLPALLR | 1-20 underlined) |
| | FGP | |
| 3 | DIQMTQSPSS LSASVGDRVT ITCKASQNVG | Humanized light chain amino |
| | SNVDWYQQKP GKAPKLLIYK ASNRYTGVPS | acid sequence of anti-Ly6E |
| | RFSGSGSGTD FTFTISSLQP EDIATYYCMQ | antibody clone 3A3.v2.D92E |
| | SETKYTFGQG TKVEIKRTVA APSVFIFPPS | (VL amino acid sequence 1- |
| | DEQLKSGTAS VVCLLNNFYP REAKVQWKVD | 106 underlined, also identified |
| | NALQSGNSQE SVTEQDSKDS TYSLSSTLTL | as SEQ ID NO. 31) |
| | SKADYEKHKV YACEVTHQGL SSPVTKSFNR | |
| | GEC | |
| 4 | DIVMTQSPTS ISISVGERVT MNCKASQNVG | Chimeric variable light chain |
| | SNVDWYQQKT GQSPELLIYK ASNRYTGVPG | amino acid sequence of anti- |
| | RFTGSGSGTD FTFTISNLQA EDLAVYYCMQ | Ly6E antibody clone 3A3 |
| | SDTKYTFGAG TKVEIKRTVA APSVFIFPPS | |
| | DEQLKSGTAS VVCLLNNFYP REAKVQWKVD | |
| | NALQSGNSQE SVTEQDSKDS TYSLSSTLTL | |
| | SKADYEKHKV YACEVTHQGL SSPVTKSFNR | |
| | GEC | |
| 5 | EVQLVESGGG LVQPGGSLKL SCAASGFTFS | Humanized heavy chain amino |
| | TVAMHWVRQA SGKGLEWVGR IRTKPVNYET | acid sequence of anti-Ly6E |
| | YYADSVKGRF TISRDDSKST VYLQMNSLKT | antibody clone 3A3 (VH amino |
| | EDTAVYYCSA PNYYDGTYSF AYWGQGTLVT | acid sequence 1-123 |
| | VSSASTKGPS VFPLAPSSKS TSGGTAALGC | underlined, also identified as |
| | LVKDYFPEPV TVSWNSGALT SGVHTFPAVL | SEQ ID NO: 32) |
| | QSSGLYSLSS VVTVPSSSLG TQTYICNVNH | |
| | KPSNTKVDKK VEPKSCDKTH TCPPCPAPEL | |
| | LGGPSVFLFP PKPKDTLMIS RTPEVTCVVV | |
| | DVSHEDPEVK FNWYVDGVEV HNAKTKPREE | |
| | QYNSTYRVVS VLTVLHQDWL NGKEYKCKVS | |
| | NKALPAPIEK TISKAKGQPR EPQVYTLPPS | |
| | REEMTKNQVS LTCLVKGFYP SDIAVEWESN | |
| | GQPENNYKTT PPVLDSDGSF FLYSKLTVDK | |
| | SRWQQGNVFS CSVMHEALHN HYTQKSLSLS | |
| | PGK | |
| 6 | AVQLVEFGGG LVQPKESLKI SCAASGFTFS | Chimeric heavy chain amino |
| | TVAMHWVRQA PGKGLEWIAR IRTKPVNYET | acid sequence of anti-Ly6E |
| | YYADSVKGRF TISRDDSKSM VYLQMDNLKT | antibody clone 3A3 |
| | EDAAMYYCSA PNYYDGTYSF AYWGQGTLVT | |
| | VSSASTKGPS VFPLAPSSKS TSGGTAALGC | |
| | LVKDYFPEPV TVSWNSGALT SGVHTFPAVL | |
| | QSSGLYSLSS VVTVPSSSLG TQTYICNVNH | |
| | KPSNTKVDKK VEPKSCDKTH TCPPCPAPEL | |
| | LGGPSVFLFP PKPKDTLMIS RTPEVTCVVV | |
| | DVSHEDPEVK FNWYVDGVEV HNAKTKPREE | |
| | QYNSTYRVVS VLTVLHQDWL NGKEYKCKVS | |
| | NKALPAPIEK TISKAKGQPR EPQVYTLPPS | |
| | REEMTKNQVS LTCLVKGFYP SDIAVEWESN | |
| | GQPENNYKTT PPVLDSDGSF FLYSKLTVDK | |
| | SRWQQGNVFS CSVMHEALHN HYTQKSLSLS | |
| | PGK | |
| 7 | KASQNVGSNVD | Humanized variable light chain |
| | | CDR 1 amino acid sequence of |
| | | anti-Ly6E antibody clone |
| | | 3A3.v2.D92E |
| 8 | KASNRYT | Humanized variable light chain |
| | | CDR 2 amino acid sequence of |
| | | anti-Ly6E antibody clone |
| | | 3A3.v2.D92E |
| 9 | MQSETKYT | Humanized variable light chain |
| | | CDR 3 amino acid sequence of |
| | | anti-Ly6E antibody clone |
| | | 3A3.v2.D92E |
| 10 | GFTFSTVAMH | Humanized variable heavy |
| | | chain CDR 1 amino acid |
| | | sequence of anti-Ly6E |
| | | antibody clone 3A3 |
| 11 | RIRTKPVNYETYYADSVKG | Humanized variable heavy |
| | | chain CDR 2 amino acid |
| | | sequence of anti-Ly6E |
| | | antibody clone 3A3 |
| 12 | SAPNYYDGTYSFAY | Humanized variable heavy |
| | | chain CDR 3 amino acid |
| | | sequence of anti-Ly6E |
| | | antibody clone 3A3 |
| 13 | ATGGGATGGTCATGTATCATCCTTTTTCTAGT | Humanized heavy chain |
| | AGCAACTGCAACCGGTGTACATTCAGAGGTGC | nucleotide sequence of anti- |
| | AGCTGGTGGAGTCTGGGGGAGGCTTGGTCCAG | Ly6E antibody clone 3A3 with |
| | CCTGGGGGGTCCCTGAAACTCTCCTGTGCAGC | signal sequence (nucleotides |
| | CTCTGGGTTCACCTTCAGTACCGTTGCTATGC | 1-57, underlined) |
| | ACTGGGTCCGCCAGGCTTCCGGGAAAGGGCTG | |
| | GAGTGGGTTGGCCGTATTAGAACCAAACCTGT | |
| | CAATTACGAGACATACTATGCTGACTCGGTGA | |
| | AAGGCAGGTTCACCATCTCCAGAGATGATTCA | |
| | AAGTCCACGGTGTATCTGCAAATGAACAGCCT | |
| | GAAAACCGAGGACACGGCCGTGTATTACTGTT | |
| | CTGCACCAAATTACTACGACGGTACTTACTCT | |
| | TTTGCCTACTGGGGCCAAGGAACCCTGGTCAC | |
| | CGTCTCGAGTGCCTCCACCAAGGGCCCATCGG | |
| | TCTTCCCCCTGGCACCCTCCTCCAAGAGCACC | |
| | TCTGGGGGCACAGCGGCCCTGGGCTGCCTGGT | |
| | CAAGGACTACTTCCCCGAGCCGGTGACGGTGT | |
| | CGTGGAACTCAGGCGCCCTGACCAGCGGCGTG | |
| | CACACCTTCCCGGCTGTCCTACAGTCCTCAGG | |
| | ACTCTACTCCCTCAGCAGCGTGGTGACTGTGC | |
| | CCTCTAGCAGCTTGGGCACCCAGACCTACATC | |
| | TGCAACGTGAATCACAAGCCCAGCAACACCAA | |
| | GGTGGACAAGAAAGTTGAGCCCAAATCTTGTG | |
| | ACAAAACTCACACATGCCCACCGTGCCCAGCA | |
| | CCTGAACTCCTGGGGGGACCGTCAGTCTTCCT | |
| | CTTCCCCCCAAAACCCAAGGACACCCTCATGA | |
| | TCTCCCGGACCCCTGAGGTCACATGCGTGGTG | |
| | GTGGACGTGAGCCACGAAGACCCTGAGGTCAA | |
| | GTTCAACTGGTACGTGGACGGCGTGGAGGTGC | |
| | ATAATGCCAAGACAAAGCCGCGGGAGGAGCAG | |
| | TACAACAGCACGTACCGTGTGGTCAGCGTCCT | |
| | CACCGTCCTGCACCAGGACTGGCTGAATGGCA | |
| | AGGAGTACAAGTGCAAGGTCTCCAACAAAGCC | |
| | CTCCCAGCCCCCATCGAGAAAACCATCTCCAA | |
| | AGCCAAAGGGCAGCCCCGAGAACCACAGGTGT | |
| | ACACCCTGCCCCCATCCCGGGAAGAGATGACC | |
| | AAGAACCAGGTCAGCCTGACCTGCCTGGTCAA | |
| | AGGCTTCTATCCCAGCGACATCGCCGTGGAGT | |
| | GGGAGAGCAATGGGCAGCCGGAGAACAACTAC | |
| | AAGACCACGCCTCCCGTGCTGGACTCCGACGG | |
| | CTCCTTCTTCCTCTACAGCAAGCTCACCGTGG | |
| | ACAAGAGCAGGTGGCAGCAGGGGAACGTCTTC | |
| | TCATGCTCCGTGATGCATGAGGCTCTGCACAA | |
| | CCACTACACGCAGAAGAGCCTCTCCCTGTCTC | |
| | CGGGTAAATGA | |
| 14 | ATGGGATGGTCATGTATCATCCTTTTTCTAGT | Humanized light chain |
| | AGCAACTGCAACCGGTGTACATTCAGACATCC | nucleotide sequence of anti- |
| | AGATGACCCAGTCTCCATCCTCCCTGTCTGCA | Ly6E antibody clone |
| | TCTGTAGGAGACAGAGTCACCATCACTTGCAA | 3A3.v2.D92E with signal |
| | GGCGAGTCAGAACGTTGGCTCCAATGTAGATT | sequence (nucleotides 1-57, |
| | GGTATCAGCAGAAACCAGGGAAAGCCCCTAAG | underlined) |
| | CTCCTGATCTACAAGGCATCCAATCGGTACAC | |
| | AGGGGTCCCATCAAGGTTCAGTGGAAGTGGAT | |
| | CTGGGACAGATTTTACTTTCACCATCAGCAGC | |
| | CTGCAGCCTGAAGATATTGCAACATATTACTG | |
| | TATGCAGTCTGAGACTAAGTACACTTTTGGCC | |
| | AGGGTACCAAGGTGGAGATCAAACGAACTGTG | |
| | GCTGCACCATCTGTCTTCATCTTCCCGCCATC | |
| | TGATGAGCAGTTGAAATCTGGAACTGCTTCTG | |
| | TTGTGTGCCTGCTGAATAACTTCTATCCCAGA | |
| | GAGGCCAAAGTACAGTGGAAGGTGGATAACGC | |
| | CCTCCAATCGGGTAACTCCCAGGAGAGTGTCA | |
| | CAGAGCAGGACAGCAAGGACAGCACCTACAGC | |
| | CTCAGCAGCACCCTGACGCTGAGCAAAGCAGA | |
| | CTACGAGAAACACAAAGTCTACGCCTGCGAAG | |
| | TCACCCATCAGGGCCTGAGCTCGCCCGTCACA | |
| | AAGAGCTTCAACAGGGGAGAGTGTTAA | |
| 15 | ATGGGATGGTCATGTATCATCCTTTTTCTAGT | Chimeric light chain nucleotide |
| | AGCAACTGCAACCGGTGTACATTCAGACATTG | sequence of anti-Ly6E |
| | TGATGACTCAGTCTCCCACATCCATTTCCATA | antibody clone 3A3 with signal |
| | TCAGTAGGAGAGAGGGTCACCATGAACTGCAA | sequence (nucleotides 1-57, |
| | GGCCAGTCAGAATGTGGGTTCTAATGTTGACT | underlined) |
| | GGTATCAACAGAAAACAGGGCAGTCTCCTGAA | |
| | CTGCTTATCTACAAGGCATCCAATCGGTACAC | |
| | TGGCGTCCCTGGTCGCTTCACAGGCAGTGGAT | |
| | CTGGAACAGATTTCACTTTCACCATCAGCAAC | |
| | TTGCAGGCTGAAGACCTGGCTGTTTATTACTG | |
| | TATGCAGTCTGACACCAAGTACACGTTTGGAG | |
| | CTGGTACCAAGGTGGAGATCAAACGAACTGTG | |
| | GCTGCACCATCTGTCTTCATCTTCCCGCCATC | |
| | TGATGAGCAGTTGAAATCTGGAACTGCTTCTG | |
| | TTGTGTGCCTGCTGAATAACTTCTATCCCAGA | |
| | GAGGCCAAAGTACAGTGGAAGGTGGATAACGC | |
| | CCTCCAATCGGGTAACTCCCAGGAGAGTGTCA | |
| | CAGAGCAGGACAGCAAGGACAGCACCTACAGC | |
| | CTCAGCAGCACCCTGACGCTGAGCAAAGCAGA | |
| | CTACGAGAAACACAAAGTCTACGCCTGCGAAG | |
| | TCACCCATCAGGGCCTGAGCTCGCCCGTCACA | |
| | AAGAGCTTCAACAGGGGAGAGTGTTAA | |
| 16 | ATGGGATGGTCATGTATCATCCTTTTTCTAGT | Chimeric heavy chain |
| | AGCAACTGCAACCGGTGTACATTCAGCGGTAC | nucleotide sequence of anti- |
| | AACTTGTTGAGTTTGGTGGAGGATTGGTGCAG | Ly6E antibody clone 3A3 with |
| | CCTAAGGAGTCATTGAAAATCTCATGTGCAGC | signal sequence (nucleotides |
| | CTCTGGATTCACCTTCAGTACTGTTGCCATGC | 1-57, underlined) |
| | ACTGGGTCCGCCAGGCTCCAGGAAAGGGTCTG | |
| | GAATGGATTGCTCGCATTAGAACTAAACCTGT | |
| | TAATTATGAAACATATTATGCTGATTCAGTGA | |
| | AAGGCAGATTCACCATCTCCCGAGATGATTCA | |
| | AAGAGCATGGTCTACCTACAAATGGATAACTT | |
| | GAAAACTGAGGACGCAGCCATGTATTACTGTT | |
| | CAGCTCCTAATTATTATGATGGTACTTATTCC | |
| | TTTGCTTACTGGGGCCAAGGCACTCTGGTAAC | |
| | TGTCTCGAGTGCCTCCACCAAGGGCCCATCGG | |
| | TCTTCCCCCTGGCACCCTCCTCCAAGAGCACC | |
| | TCTGGGGGCACAGCGGCCCTGGGCTGCCTGGT | |
| | CAAGGACTACTTCCCCGAGCCGGTGACGGTGT | |
| | CGTGGAACTCAGGCGCCCTGACCAGCGGCGTG | |
| | CACACCTTCCCGGCTGTCCTACAGTCCTCAGG | |
| | ACTCTACTCCCTCAGCAGCGTGGTGACTGTGC | |
| | CCTCTAGCAGCTTGGGCACCCAGACCTACATC | |
| | TGCAACGTGAATCACAAGCCCAGCAACACCAA | |
| | GGTGGACAAGAAAGTTGAGCCCAAATCTTGTG | |
| | ACAAAACTCACACATGCCCACCGTGCCCAGCA | |
| | CCTGAACTCCTGGGGGGACCGTCAGTCTTCCT | |
| | CTTCCCCCCAAAACCCAAGGACACCCTCATGA | |
| | TCTCCCGGACCCCTGAGGTCACATGCGTGGTG | |
| | GTGGACGTGAGCCACGAAGACCCTGAGGTCAA | |
| | GTTCAACTGGTACGTGGACGGCGTGGAGGTGC | |
| | ATAATGCCAAGACAAAGCCGCGGGAGGAGCAG | |
| | TACAACAGCACGTACCGTGTGGTCAGCGTCCT | |
| | CACCGTCCTGCACCAGGACTGGCTGAATGGCA | |
| | AGGAGTACAAGTGCAAGGTCTCCAACAAAGCC | |
| | CTCCCAGCCCCCATCGAGAAAACCATCTCCAA | |
| | AGCCAAAGGGCAGCCCCGAGAACCACAGGTGT | |
| | ACACCCTGCCCCCATCCCGGGAAGAGATGACC | |
| | AAGAACCAGGTCAGCCTGACCTGCCTGGTCAA | |
| | AGGCTTCTATCCCAGCGACATCGCCGTGGAGT | |
| | GGGAGAGCAATGGGCAGCCGGAGAACAACTAC | |
| | AAGACCACGCCTCCCGTGCTGGACTCCGACGG | |
| | CTCCTTCTTCCTCTACAGCAAGCTCACCGTGG | |
| | ACAAGAGCAGGTGGCAGCAGGGGAACGTCTTC | |
| | TCATGCTCCGTGATGCATGAGGCTCTGCACAA | |
| | CCACTACACGCAGAAGAGCCTCTCCCTGTCTC | |
| | CGGGTAAATGA | |
| 17 | DIQMTQSPSS LSASVGDRVT ITCKASQNVG | Humanized light chain amino |
| | SNVDWYQQKP GKSPKLLIYK ASNRYTGVPS | acid sequence of anti-Ly6E |
| | RFSGSGSGTD FTFTISSLQP EDIATYYCMQ | antibody clone 3A3 |
| | SDTKYTFGQG TKVEIKRTVA APSVFIFPPS | |
| | DEQLKSGTAS VVCLLNNFYP REAKVQWKVD | |
| | NALQSGNSQE SVTEQDSKDS TYSLSSTLTL | |
| | SKADYEKHKV YACEVTHQGL SSPVTKSFNR | |
| | GEC | |
| 18 | ATGGGATGGTCATGTATCATCCTTTTTCTAGT | Humanized light chain |
| | AGCAACTGCAACCGGTGTACATTCAGACATCC | nucleotide sequence of anti- |
| | AGATGACCCAGTCTCCATCCTCCCTGTCTGCA | Ly6E antibody clone 3A3 with |
| | TCTGTAGGAGACAGAGTCACCATCACTTGCAA | signal sequence (nucleotides |
| | GGCGAGTCAGAACGTTGGCTCCAATGTAGATT | 1-57, underlined) |
| | GGTATCAGCAGAAACCAGGGAAATCCCCTAAG | |
| | CTCCTGATCTACAAGGCATCCAATCGGTACAC | |
| | AGGGGTCCCATCAAGGTTCAGTGGAAGTGGAT | |
| | CTGGGACAGATTTTACTTTCACCATCAGCAGC | |
| | CTGCAGCCTGAAGATATTGCAACATATTACTG | |
| | TATGCAGTCTGATACTAAGTACACTTTTGGCC | |
| | AGGGTACCAAGGTGGAGATCAAACGAACTGTG | |
| | GCTGCACCATCTGTCTTCATCTTCCCGCCATC | |
| | TGATGAGCAGTTGAAATCTGGAACTGCTTCTG | |
| | TTGTGTGCCTGCTGAATAACTTCTATCCCAGA | |
| | GAGGCCAAAGTACAGTGGAAGGTGGATAACGC | |
| | CCTCCAATCGGGTAACTCCCAGGAGAGTGTCA | |
| | CAGAGCAGGACAGCAAGGACAGCACCTACAGC | |
| | CTCAGCAGCACCCTGACGCTGAGCAAAGCAGA | |
| | CTACGAGAAACACAAAGTCTACGCCTGCGAAG | |
| | TCACCCATCAGGGCCTGAGCTCGCCCGTCACA | |
| | AAGAGCTTCAACAGGGGAGAGTGTTA | |
| 19 | DIQMTQSPSS LSASVGDRVT ITCKASQNVG | Humanized light chain amino |
| | SNVDWYQQKP GKSPKLLIYK ASNRYTGVPS | acid sequence of anti-Ly6E |
| | RFSGSGSGTD FTFTISSLQP EDIATYYCMQ | antibody clone 3A3.K149C |
| | SDTKYTFGQG TKVEIKRTVA APSVFIFPPS | |
| | DEQLKSGTAS VVCLLNNFYP REAKVQWCVD | |
| | NALQSGNSQE SVTEQDSKDS TYSLSSTLTL | |
| | SKADYEKHKV YACEVTHQGL SSPVTKSFNR | |
| | GEC | |
| 20 | ATGGGATGGTCATGTATCATCCTTTTTCTAGT | Humanized light chain |
| | AGCAACTGCAACCGGTGTACATTCAGACATCC | nucleotide sequence of anti- |
| | AGATGACCCAGTCTCCATCCTCCCTGTCTGCA | Ly6E antibody clone |
| | TCTGTAGGAGACAGAGTCACCATCACTTGCAA | 3A3.K149C with signal |
| | GGCGAGTCAGAACGTTGGCTCCAATGTAGATT | sequence (nucleotides 1-57, |
| | GGTATCAGCAGAAACCAGGGAAATCCCCTAAG | underlined) |
| | CTCCTGATCTACAAGGCATCCAATCGGTACAC | |
| | AGGGGTCCCATCAAGGTTCAGTGGAAGTGGAT | |
| | CTGGGACAGATTTTACTTTCACCATCAGCAGC | |
| | CTGCAGCCTGAAGATATTGCAACATATTACTG | |
| | TATGCAGTCTGATACTAAGTACACTTTTGGCC | |
| | AGGGTACCAAGGTGGAGATCAAACGAACTGTG | |
| | GCTGCACCATCTGTCTTCATCTTCCCGCCATC | |
| | TGATGAGCAGTTGAAATCTGGAACTGCTTCTG | |
| | TTGTGTGCCTGCTGAATAACTTCTATCCCAGA | |
| | GAGGCCAAAGTACAGTGGTGCGTGGATAACGC | |
| | CCTCCAATCGGGTAACTCCCAGGAGAGTGTCA | |
| | CAGAGCAGGACAGCAAGGACAGCACCTACAGC | |
| | CTCAGCAGCACCCTGACGCTGAGCAAAGCAGA | |
| | CTACGAGAAACACAAAGTCTACGCCTGCGAAG | |
| | TCACCCATCAGGGCCTGAGCTCGCCCGTCACA | |
| | AAGAGCTTCAACAGGGGAGAGTGTTAA | |
| 21 | MKIFLPVLLAALLGVERASSLMCFSCLNQKSN | RHESUS Ly6E amino acid |
| | LYCLKPTICSDQDNYCVTVSTSAGIGNLVTFG | sequence with signal sequence |
| | HSLSKTCSPACPLPEGINVGVASMGISCCQSF | (amino acids 1-20, underlined) |
| | LCNFSAADGGLRASATLLGAGLLLSLLPALLR | |
| | FGP | |
| 22 | MSATSNMRVFLPVLLAALLGMEQVHSLMCFSC | MOUSE Ly6E amino acid |
| | TDQKNNINCLWPVSCQEKDHYCITLSAAAGFG | sequence with signal sequence |
| | NVNLGYTLNKGCSPICPSENVNLNLGVASVNS | (amino acids 1-26, underlined) |
| | YCCQSSFCNFSAAGLGLRASIPLLGLGLLLSL | |
| | LALLQLSP | |
| 23 | MSAASSMRVFLPVLLAALLGVEQVHSLMCFSC | RAT Ly6E amino acid |
| | TDQKNNINCLWPVSCSSTDNYCITLSAAAGFG | sequence with signal sequence |
| | NVNLGYTLNKGCSPTCPRENININLGVASVNS | (amino acids 1-26, underlined) |
| | YCCQSSFCNFSTAGLGLRASIPLLGLGLLLSL | |
| | LAVLRLSP | |
| 24 | LMCFSCLNQKSNLYCLKPTICSDQDNYCVTVS | Mature HUMAN Ly6E amino |
| | ASAGIGNLVTFGHSLSKTCSPACPIPEGVNVG | acid sequence (without signal |
| | VASMGISCCQSFLCNFSAADGGLRASVTLLGA | sequence) |
| | GLLLSLLPALLRFGP | |
| 25 | LMCFSCLNQKSNLYCLKPTICSDQDNYCVTVS | Mature CYNOMOLOGOUS |
| | TSAGIGNLVTFGHSLSKTCSPACPLPEGINVG | Ly6E amino acid sequence |
| | VASMGISCCQSFLCNFSAADGGLRASATLLGA | (without signal sequence) |
| | GLLLSLLPALLRFGP | |
| 26 | LMCFSCLNQKSNLYCLKPTICSDQDNYCVTVS | Mature RHESUS Ly6E amino |
| | TSAGIGNLVTFGHSLSKTCSPACPLPEGINVG | acid sequence (without signal |
| | VASMGISCCQSFLCNFSAADGGLRASATLLGA | sequence) |
| | GLLLSLLPALLRFGP | |
| 27 | LMCFSCTDQKNNINCLWPVSCQEKDHYCITLS | Mature MOUSE Ly6E amino |
| | AAAGFGNVNLGYTLNKGCSPICPSENVNLNLG | acid sequence (without signal |
| | VASVNSYCCQSSFCNFSAAGLGLRASIPLLGL | sequence) |
| | GLLLSLLALLQLSP | |
| 28 | LMCFSCTDQKNNINCLWPVSCSSTDNYCITLS | Mature RAT Ly6E amino acid |
| | AAAGFGNVNLGYTLNKGCSPTCPRENININLG | sequence (without signal |
| | VASVNSYCCQSSFCNFSTAGLGLRASIPLLGL | sequence) |
| | GLLLSLLAVLRLSP | |
| 29 | DIQMTQSPSS LSASVGDRVT ITCKASQNVG | Humanized light chain amino |
| | SNVDWYQQKP GKAPKLLIYK ASNRYTGVPS | acid sequence of anti-Ly6E |
| | RFSGSGSGTD FTFTISSLQP EDIATYYCMQ | antibody clone 3A3.v2.D92S |
| | SSTKYTFGQG TKVEIKRTVA APSVFIFPPS | |
| | DEQLKSGTAS VVCLLNNFYP REAKVQWKVD | |
| | NALQSGNSQE SVTEQDSKDS TYSLSSTLTL | |
| | SKADYEKHKV YACEVTHQGL SSPVTKSFNR | |
| | GEC | |
| 30 | ATGGGATGGTCATGTATCATCCTTTTTCTAGT | Humanized light chain |
| | AGCAACTGCAACCGGTGTACATTCAGACATCC | nucleotide sequence of anti- |
| | AGATGACCCAGTCTCCATCCTCCCTGTCTGCA | Ly6E antibody clone |
| | TCTGTAGGAGACAGAGTCACCATCACTTGCAA | 3A3.v2.D92S |
| | GGCGAGTCAGAACGTTGGCTCCAATGTAGATT | |
| | GGTATCAGCAGAAACCAGGGAAAGCCCCTAAG | |
| | CTCCTGATCTACAAGGCATCCAATCGGTACAC | |
| | AGGGGTCCCATCAAGGTTCAGTGGAAGTGGAT | |
| | CTGGGACAGATTTTACTTTCACCATCAGCAGC | |
| | CTGCAGCCTGAAGATATTGCAACATATTACTG | |
| | TATGCAGTCTTCTACTAAGTACACTTTTGGCC | |
| | AGGGTACCAAGGTGGAGATCAAACGAACTGTG | |
| | GCTGCACCATCTGTCTTCATCTTCCCGCCATC | |
| | TGATGAGCAGTTGAAATCTGGAACTGCTTCTG | |
| | TTGTGTGCCTGCTGAATAACTTCTATCCCAGA | |
| | GAGGCCAAAGTACAGTGGAAGGTGGATAACGC | |
| | CCTCCAATCGGGTAACTCCCAGGAGAGTGTCA | |
| | CAGAGCAGGACAGCAAGGACAGCACCTACAGC | |
| | CTCAGCAGCACCCTGACGCTGAGCAAAGCAGA | |
| | CTACGAGAAACACAAAGTCTACGCCTGCGAAG | |
| | TCACCCATCAGGGCCTGAGCTCGCCCGTCACA | |
| | AAGAGCTTCAACAGGGGAGAGTGTTAAGAAAC | |
| | AGCTATGACATGATTACGAATTAA | |
| 31 | DIQMTQSPSS LSASVGDRVT ITCKASQNVG | Humanized variable light chain |
| | SNVDWYQQKP GKAPKLLIYK ASNRYTGVPS | amino acid sequence of anti- |
| | RFSGSGSGTD FTFTISSLQP EDIATYYCMQ | Ly6E antibody clone |
| | SETKYTFGQG TKVEIK | 3A3.v2.D92E |
| 32 | EVQLVESGGG LVQPGGSLKL SCAASGFTFS | Humanized variable heavy |
| | TVAMHWVRQA SGKGLEWVGR IRTKPVNYET | chain amino acid sequence of |
| | YYADSVKGRF TISRDDSKST VYLQMNSLKT | anti-Ly6E antibody clone 3A3 |
| | EDTAVYYCSA PNYYDGTYSF AYWGQGTLVT | |
| | VSS | |
| 33 | EVQLVESGGG LVQPGGSLKL SCAASGFTFS | Humanized heavy chain amino |
| | TVAMHWVRQA SGKGLEWVGR IRTKPVNYET | acid sequence of anti-Ly6E |
| | YYADSVKGRF TISRDDSKST VYLQMNSLKT | antibody clone 3A3, without |
| | EDTAVYYCSA PNYYDGTYSF AYWGQGTLVT | C-term lysine (K) |
| | VSSASTKGPS VFPLAPSSKS TSGGTAALGC | |
| | LVKDYFPEPV TVSWNSGALT SGVHTFPAVL | |
| | QSSGLYSLSS VVTVPSSSLG TQTYICNVNH | |
| | KPSNTKVDKK VEPKSCDKTH TCPPCPAPEL | |
| | LGGPSVFLFP PKPKDTLMIS RTPEVTCVVV | |
| | DVSHEDPEVK FNWYVDGVEV HNAKTKPREE | |
| | QYNSTYRVVS VLTVLHQDWL NGKEYKCKVS | |
| | NKALPAPIEK TISKAKGQPR EPQVYTLPPS | |
| | REEMTKNQVS LTCLVKGFYP SDIAVEWESN | |
| | GQPENNYKTT PPVLDSDGSF FLYSKLTVDK | |
| | SRWQQGNVFS CSVMHEALHN HYTQKSLSLS | |
| | PG | |
| 34 | AVQLVEFGGG LVQPKESLKI SCAASGFTFS | Chimeric heavy chain amino |
| | TVAMHWVRQA PGKGLEWIAR IRTKPVNYET | acid sequence of anti-Ly6E |
| | YYADSVKGRF TISRDDSKSM VYLQMDNLKT | antibody clone 3A3, without |
| | EDAAMYYCSA PNYYDGTYSF AYWGQGTLVT | C-term lysine (K) |
| | VSSASTKGPS VFPLAPSSKS TSGGTAALGC | |
| | LVKDYFPEPV TVSWNSGALT SGVHTFPAVL | |
| | QSSGLYSLSS VVTVPSSSLG TQTYICNVNH | |
| | KPSNTKVDKK VEPKSCDKTH TCPPCPAPEL | |
| | LGGPSVFLFP PKPKDTLMIS RTPEVTCVVV | |
| | DVSHEDPEVK FNWYVDGVEV HNAKTKPREE | |
| | QYNSTYRVVS VLTVLHQDWL NGKEYKCKVS | |
| | NKALPAPIEK TISKAKGQPR EPQVYTLPPS | |
| | REEMTKNQVS LTCLVKGFYP SDIAVEWESN | |
| | GQPENNYKTT PPVLDSDGSF FLYSKLTVDK | |
| | SRWQQGNVFS CSVMHEALHN HYTQKSLSLS | |
| | PG | |

### III. EXAMPLES

The following are examples of methods and compositions of the invention. It is understood that various other embodiments may be practiced, given the general description provided above.

### Example 1—Virus-Like-Particle (VLP) Generation and Characterization

Expi293 cells were co-transfected with a mammalian expression construct encoding the full-length Ly6E and a mammalian expression construct encoding for MLGag. Seven days post-transfection, VLPs were purified from the supernatant using ultracentrifugation as previously described (see, e.g., Théry C., et al., “Isolation and Characterization of Exosomes from Cell Culture Supernatants and Biological Fluids,” Curr Protoc Cell Biology 2006; 30:3.22.1-3.22.29, and WO2021/247457). VLP concentrations were measured using a Bradford assay. Incorporation of Ly6E was confirmed by ELISA. Nunc Maxisorp ELISA plates (Thermo Scientific) were coated with various concentrations of VLPs diluted in coating buffer (50 mM carbonate, pH 9.6) at 4° C. overnight. The plates were washed with wash buffer (1×PBS with 0.05% Tween20) and then blocked with ELISA assay diluent (PBS/0.5% BSA/0.05% polysorbate 20) at room temperature for 1 hour. Plates were then incubated at room temperature for 1 hour with anti-Ly6E (9B12; 1 g/ml diluted in ELISA assay diluent). The plates were washed with wash buffer and the bound antibody was detected with goat anti-rat horse-radish peroxidase secondary antibody (Jackson Immunol Lab). The plates were incubated at room temperature for 30 minutes, washed with wash buffer and developed with TMB solution (Surmodics, USA). Plates were read at 630 nm.

### Example 2—VLP Immunization

Animals used in these studies were maintained in an AAALAC accredited animal facility. All experiments were performed in compliance with Genentech's Institutional Animal Care and Use Committee and OLAW Guidelines. Approval of the study design was obtained from the Genentech Institutional Animal Care and Use Committee prior to the start of this work.

Sprague Dawley rats (Charles River Laboratories, Hollister, CA) were immunized with Ly6E VLPs in PBS along with Ribi adjuvant (Sigma). The rats were then boosted three times with additional Ly6E VLPs, every 2 weeks. This was followed by four injections of a plasmid encoding for Ly6E cDNA via Genegun over 2 weeks. DNA/gold particle bullets are prepared essentially as previously described. See, e.g., Tang et al., Nature 1992; 356:152-4, and Hansen et al., Sci Rep-uk 2016; 6:21925.

Each bullet for DNA was prepared to contain a total of 1 g of DNA coated onto 0.5 mg of gold particles (BioRad, Cat. #1652264). Bullets were stored at 4° C. in the dark in the presence of desiccant pellets.

pAbs were purified by Protein A and assayed by FACS to identify antibodies binding to cell-surface Ly6E. To generate monoclonal antibodies, hybridoma fusions were performed as previously described except with a myeloma partner SP2ab that enables surface display of IgG cell. See, e.g., Price et al., J Immunol Methods 2009; 343:28-41. Vij et al., Sci Rep-uk 2018; 8:7136. Hybridoma supernatants were harvested and IgG was purified from supernatants using MabSelect SuRe (GE Healthcare, Piscataway, NJ, USA). Anti-Ly6E hybridomas were identified by ELISA and FACS screening. The variable light chain and variable heavy chain sequences of anti-Ly6E hybridoma were determined using 5′ RACE followed by sequencing of the PCR amplified products. A clone showing promising binding was cloned and expressed as a rat-human chimeric antibody (SEQ ID NOs. 4 and 6). Purified protein was taken forward for further analysis.

### Example 3—Characterization of Anti-Ly6E

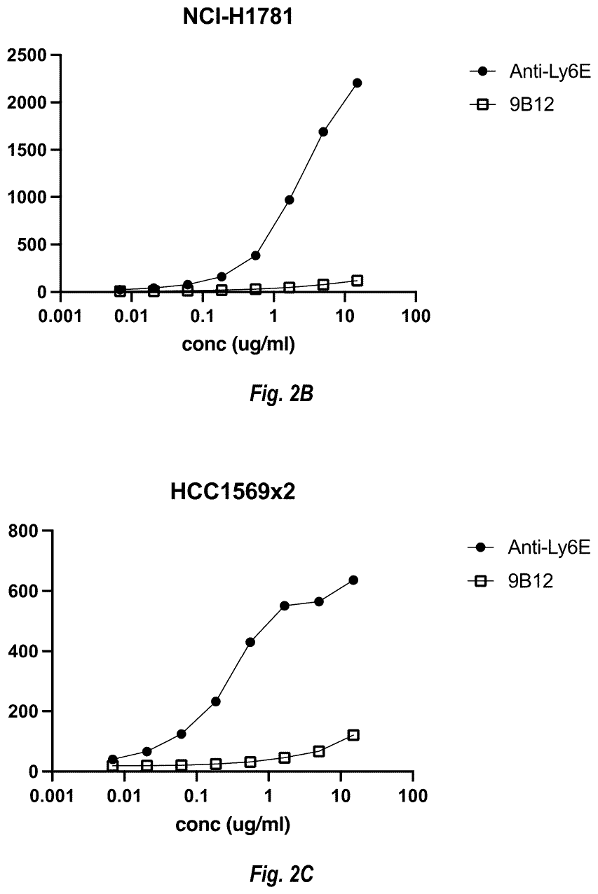

Chimeric anti-Ly6E was compared to a previously described anti-Ly6E antibody (9B12) (Asundi et al., Clin. Canc. Res. 2015, 3252-3262) for binding to cells expressing Ly6E. Cell lines were cultured as recommended by the ATCC. Prior to staining adherent lines were released by Accutase treatment, all cells were filtered and equilibrated in BD Stain buffer (BSA). Cells were stained with primary antibodies at indicated concentrations for 1 h at 4° C., washed ×3, stained with secondary Ab (Jackson ImmunoResearch 109-606-003) (1:100) for 1 h at 4° C., washed, stained with Fixable Viability Dye eFluor 780 (1:1000) for 1 h at 4° C., and washed ×2. Samples were fixed in 1% PFA (PBS) prior to analysis. Samples were assessed on a BD LSRFortessa. Following exclusion of dead cells, singlets were assessed for antibody binding using FlowJo software (FIGS. 2A-2C). In all three tested cell lines anti-Ly6E shows enhanced binding compared to 9B12.

### Example 4—Ly6E Humanization and Reformatting

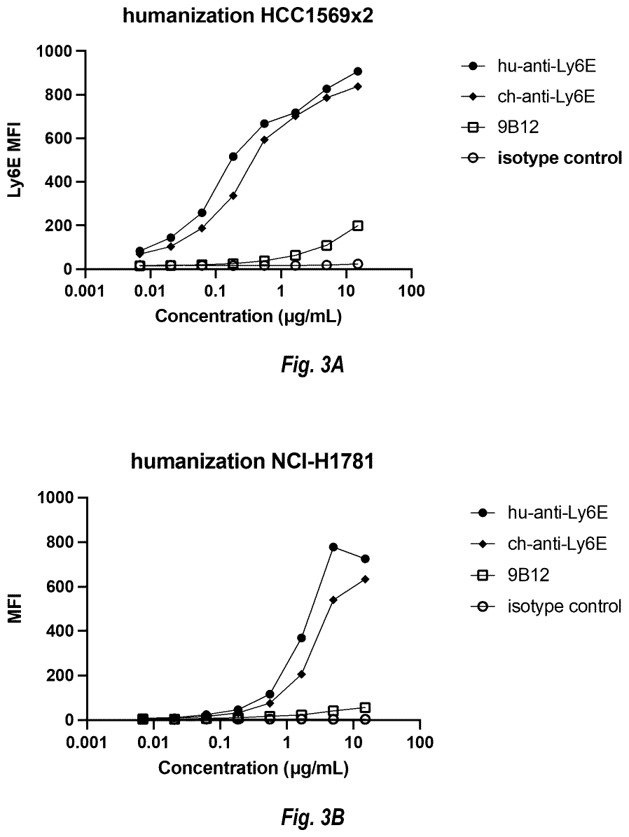

The Ly6E antibody was humanized by grafting the HC and LC CDRs into human germline frameworks (IGHV3-73*01/IGHJ1*01 and IGKV1-33*01/IGKJ2*01 respectively) and iterating between human and rat residues at Vernier positions to maximize binding while minimizing non-human residues. The binding of the humanized anti-Ly6E (comprising SEQ ID NOs. 3 and 5) was compared to the chimeric anti-Ly6E (comprising SEQ ID NOs. 4 and 6), 9B12, and an isotype control (FIGS. 3A-3B). Against two cell lines, the humanized version showed similar binding to chimeric anti-Ly6E and enhanced binding versus 9B12. Cysteine residues were incorporated at previously described positions to enable generation on THIOMAB® antibodies (Ohri et al., Bioconj Chem. 2018, 473-485). Humanized VHs were cloned into knob-into-hole backbones to generate bispecific antibodies (Ridgeway et al., Prot. Eng. 1996, 617-621).

### Example 5—Characterization and Optimization of Anti-Ly6E Stability

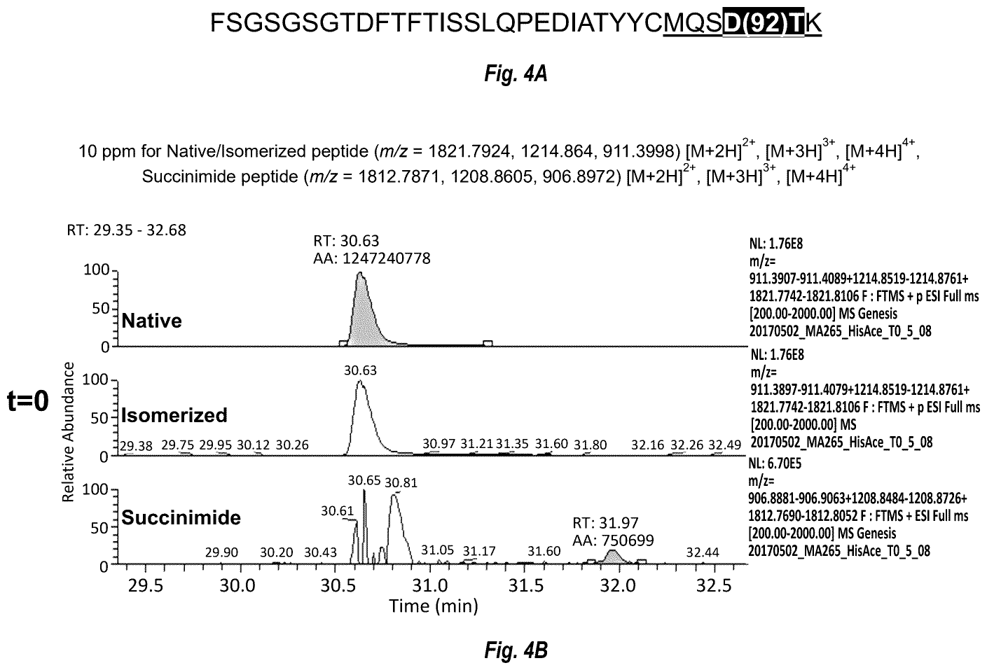

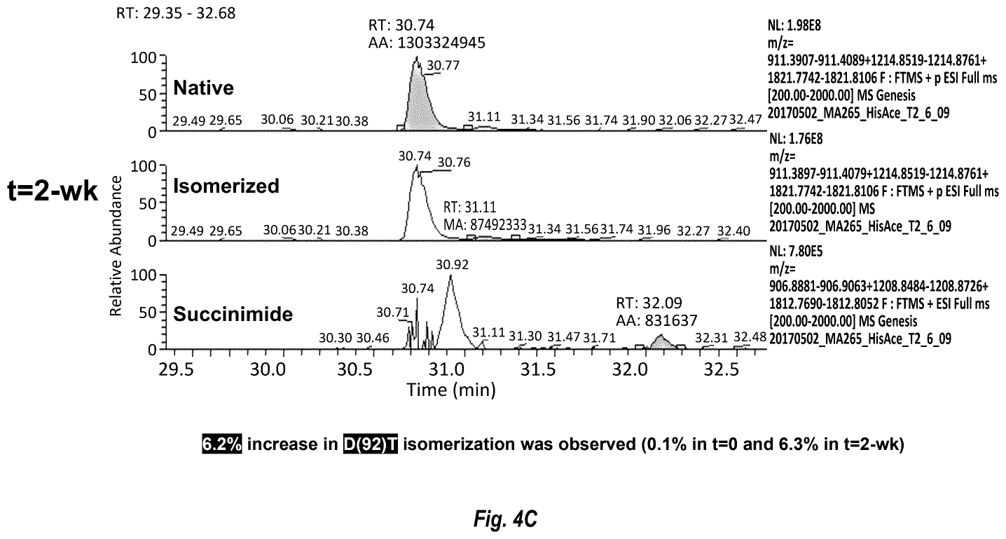

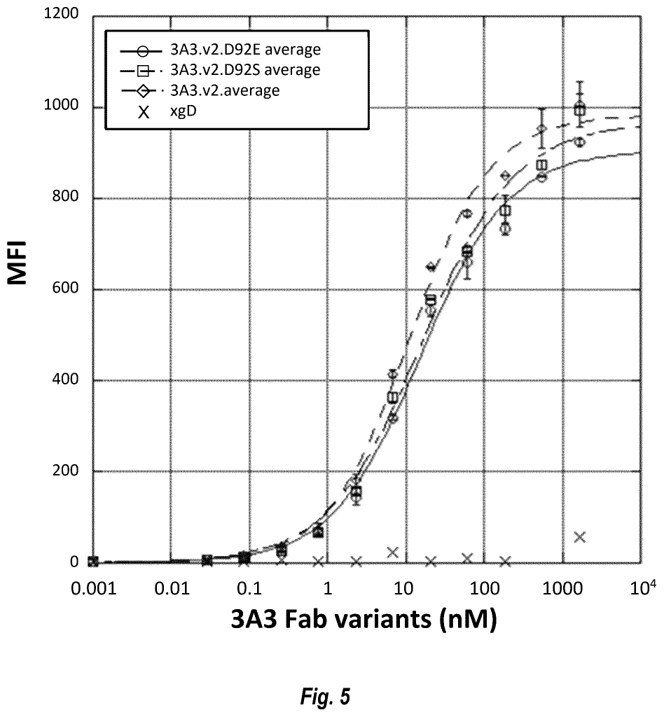

To evaluate potential manufacturing liabilities, anti-Ly6E was incubated in low ionic strength histidine-acetate buffer pH 5.5 at 40 degrees Celsius for 2 weeks and analyzed by peptide mapping essentially as previously described (Xu et al. Mol. Pharmaceutics 2018, 4529-4537). This thermal stress resulted in a 6.2% increase in the amount of isomerized light chain (LC) D92 (FIGS. 4A-4C). Mutations were incorporated to remove the problematic aspartic acids. LC D92S (SEQ ID NO.: 29) and LC D92E (SEQ ID NO.: 3) variants were generated, proteolyzed to Fabs to generate monovalent binders, and assessed for binding to NCI-H1781 cells by flow cytometry as described above (FIG. 5). Both variants showed similar binding to humanized anti-Ly6E. Notably, the Fv charges of the variants are 7.5 LC D92S and 6.5 for LC D92E. D92E was selected as the preferred mutant as its charge profile is better aligned with predictive methods for normal PK (Hotzel et al., mAbs 2012, 753-760).

### Example 6—Generation of Anti-Ly6E Antibody-Drug Conjugates

Briefly, a cysteine residue was engineered at desired position of heavy chain and/or light chain of antibody targeted to Her2, Ly6E, and CD33 antigen to produce its THIOMAB variants. The THIOMAB antibodies were conjugated to linker drugs as described previously. See Junutula et al., Nat Biotechnol 2008, 26:925-932. Briefly, the antibody was reduced in presence of hundred-fold molar excess DTT (Calbiochem) overnight. The reducing agent and the cysteine and glutathione blocks were purified away using HiTrap SP-HP column (GE Healthcare). The antibody was reoxidized in presence of fifteen-fold molar excess dhAA (MP Biomedical) for 2.5 hours. The formation of interchain disulfide bonds was monitored by LC/MS. Ten-fold molar excess linker drug over protein was incubated in presences of 15% DMF with the activated THIOMAB antibodies for 2 or 3 hours. The antibody drug conjugate was purified on HiTrap SP-HP column (GE Healthcare) to remove excess linker drug. If there was still aggregation of more than 5% present by analytical SEC then it was purified over Hi Load Superdex 200 pg 16/600 column (GE Healthcare) using 20 mM Histidine-acetate, 150 mM NaCl, pH5.5 as running buffer. The number of conjugated linker drug molecules per THIOMAB antibody was quantified by LC/MS analysis. Purity was assessed by size exclusion chromatography.

### Mass Spectrometric Analysis

LC/MS analysis was performed on a 6530 Accurate-Mass Quadrupole Time-of-Flight (Q-TOF) LC/MS (Agilent Technologies). Samples were chromatographed on a PRLP-S, 1000 Å, 8 μm (50 mm 2.1 mm, Agilent Technologies) heated to 80° C. A linear gradient from 30-60% B in 4.3 minutes (solvent A, 0.05% TFA in water; solvent B, 0.04% TFA in acetonitrile) was used and the eluent was directly ionized using the electrospray source. Data was collected and deconvoluted using the Agilent Mass Hunter qualitative analysis software. Before LC/MS analysis, antibody drug conjugate was treated with DTT for 30 minutes at 15 mM, pH 8.0, and 37° C. to produce the HC and the LC portion for ease of analysis. The drug to antibody ratio (DAR) was calculated using the abundance of the ions present in LC/MS deconvoluted results. The peaks were identified using LC/MS.

The LC/MS analysis of the reduced conjugates showed that antibody drug conjugates had a range of 1.8-2 drugs per antibody for all of the conjugates.

### Example 7—Characterization of Anti-Ly6E as a SN36325 Antibody-Drug Conjugate

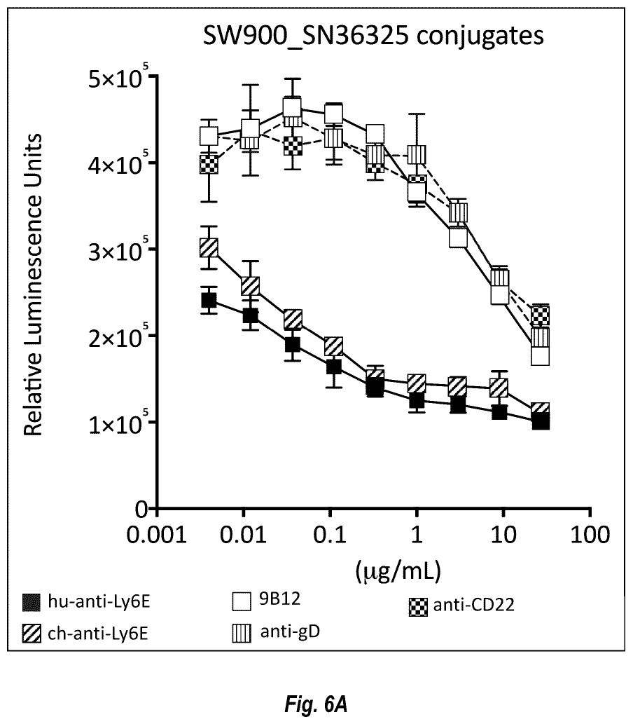

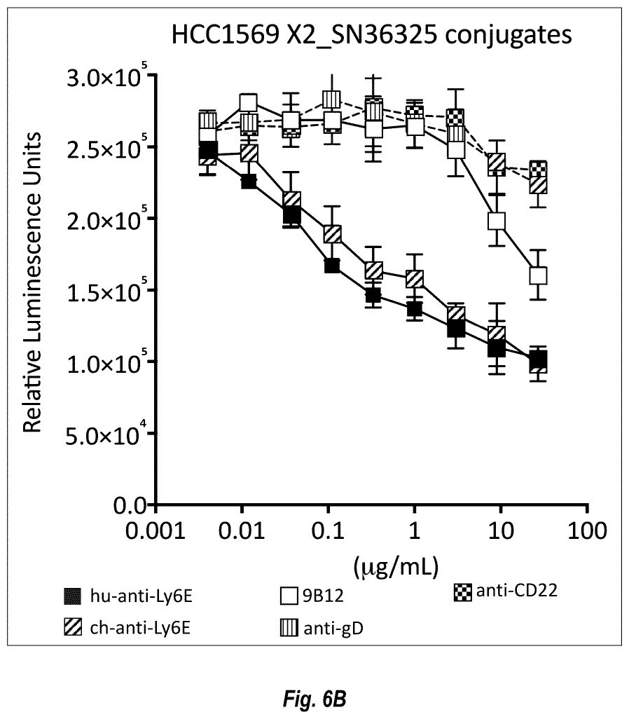

Cells were seeded in 384-well plates, grown for 24 h, and treated with the indicated ADCs including conjugates to isotype controls (anti-gD, and anti-CD22) and 9B12. After 5 days of continuous ADC incubation, the cell viability was determined using Promega CellTiter-Glo® luminescent reagent. The luminescent intensity was measured using a PerkinElmer EnVision® reader (FIGS. 6A-6B). The relative cell viability was calculated by normalizing to non-drug treatment control. Anti-Ly6E conjugates show more potent killing against both HCC1569x2 and SW900 cell lines than isotype control- and 9B12-conjugates. Minimal differences were seen between the chimeric and humanized versions of anti-Ly6E.

### Example 8—Synthesis of (maleimide-sq-ala)-PBD Linker-Drug

Scheme for Synthesis of (maleimide-sq-ala)-PBD linker-drug:

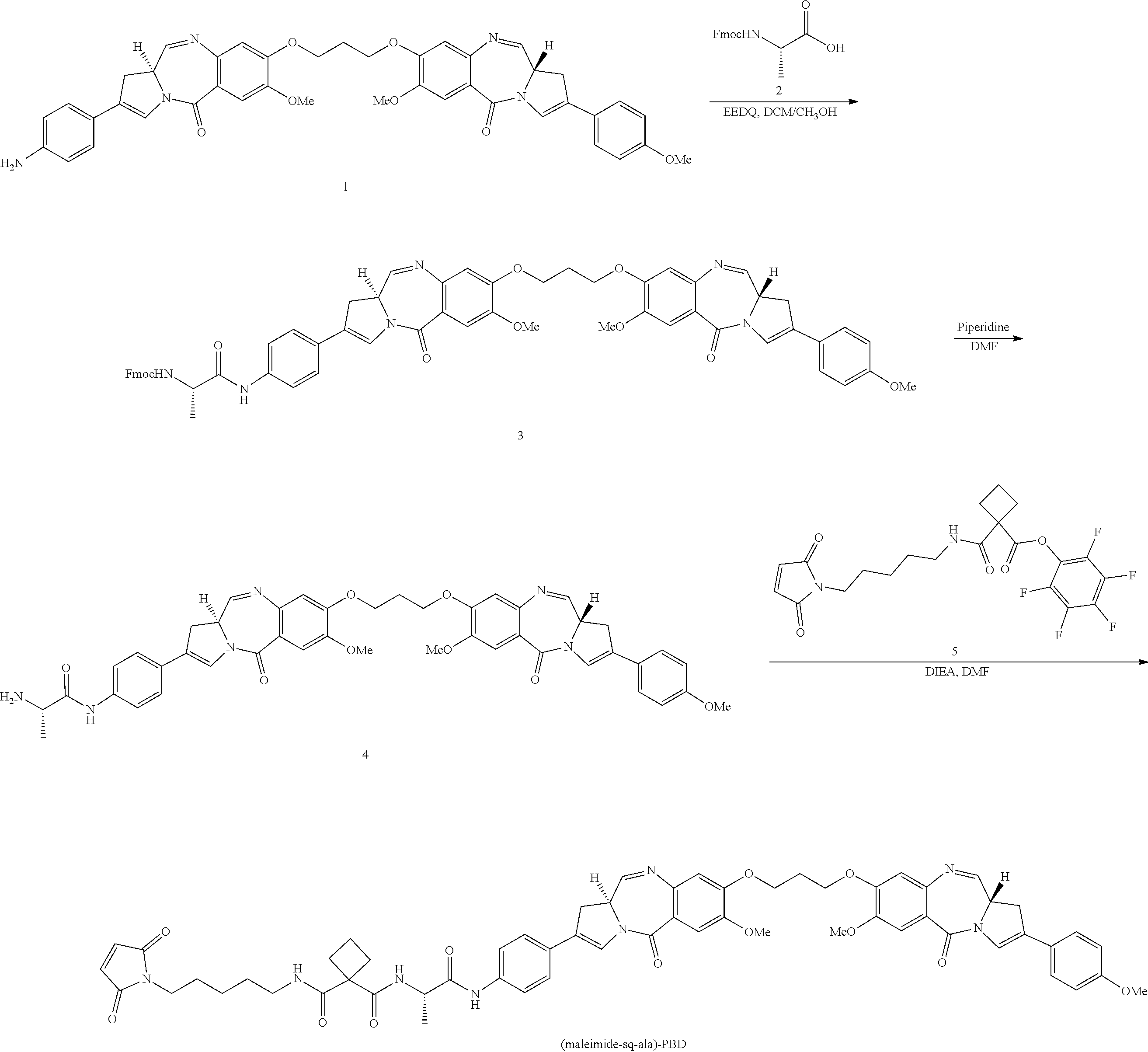

The maleimide-sq-ala linker is conjugated to the PBD payload in two steps, first installing the alanine and then conjugating the maleimide-sq through INT5, an activated maleimide-sq-ester. The linker in this example has a 5-carbon spacer between the maleimide and the sq.

### Scheme of Synthesis of INT5-Precursor:

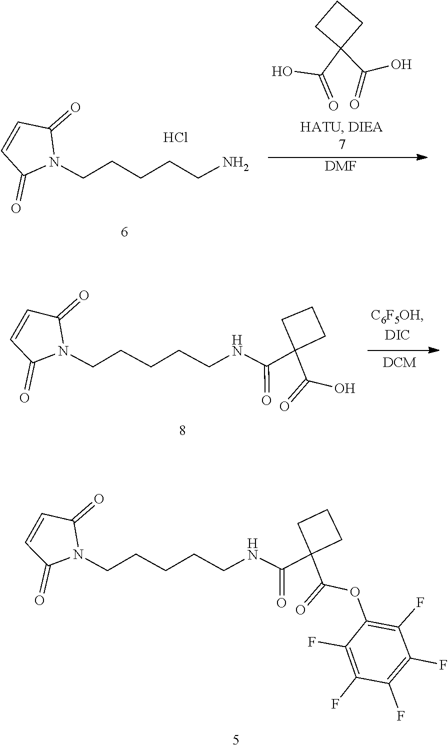

### General Procedure for Preparation of Compound 3:

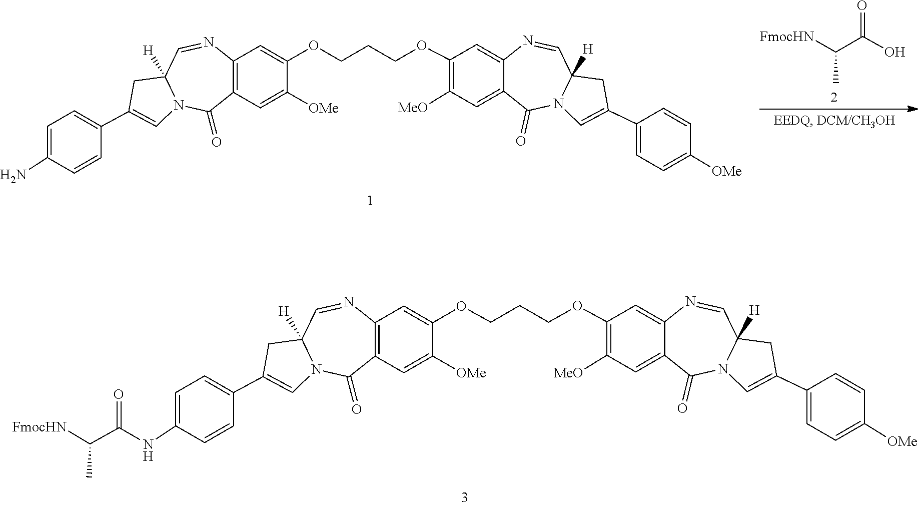

To a solution of compound 2 (386 mg, 1.24 mmol) in DCM (20 mL) and Methanol (2 mL) was added EEDQ (306 mg, 1.24 mmol). The mixture was stirred at 20° C. for 5 min. Then to above solution was added compound 1 (300.0 mg, 0.41 mmol; available commercially, e.g., BOC Sciences “PBD dimer—CAS 1222490-34-7” at www.bocsci.com/product/pbd-dimer-cas-1222490-34-7-477053.html. The reaction mixture was stirred at 20° C. for 12 hours. The mixture was concentrated and methyl tert-butyl ether (50 mL) was added. The solid was collected and washed with methyl tert-butyl ether (50 mL×2) to give the crude product compound 3 (400 mg, 60%) as a yellow solid, which was used for next step directly. LCMS (5-95, AB, 1.5 min): R_{T}=0.838 min, m/z=1019.6[M+H]⁺.

### General Procedure for Preparation of Compound 4:

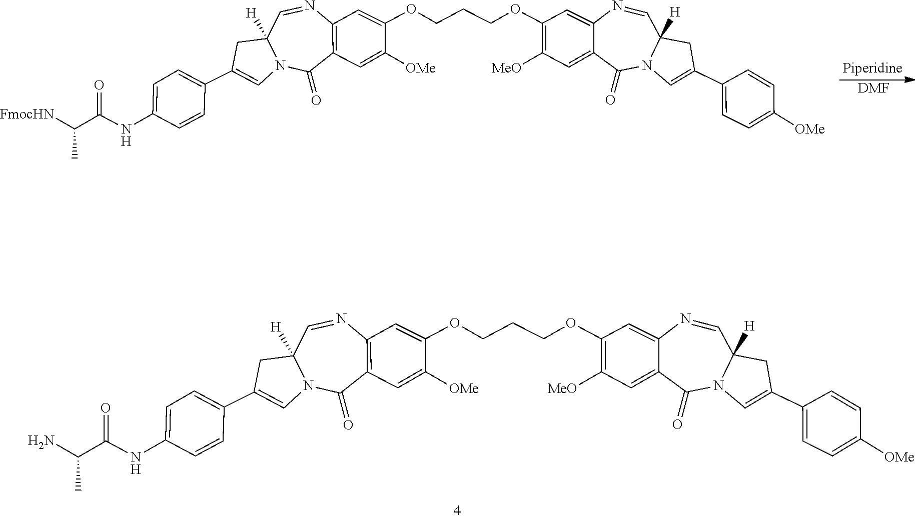

To a solution of compound 3 (70.0 mg, 0.07 mmol) in DMF (2 mL) was added piperidine (0.01 mL, 0.14 mmol). The mixture was stirred at 20° C. for 30 min. The mixture was concentrated and methyl tert-butyl ether (40 mL) was added. The mixture was filtered to give a solid, which was washed with methyl tert-butyl ether (40 mL×2) to give the crude compound 4 (40 mg, 73%) as a yellow solid. LCMS (5-95, AB, 1.5 min): RT=0.765 min, m/z=797.4 [M+H]⁺.

General Procedure for Coupling of INT5-Precursor to ala-PBD Payload:

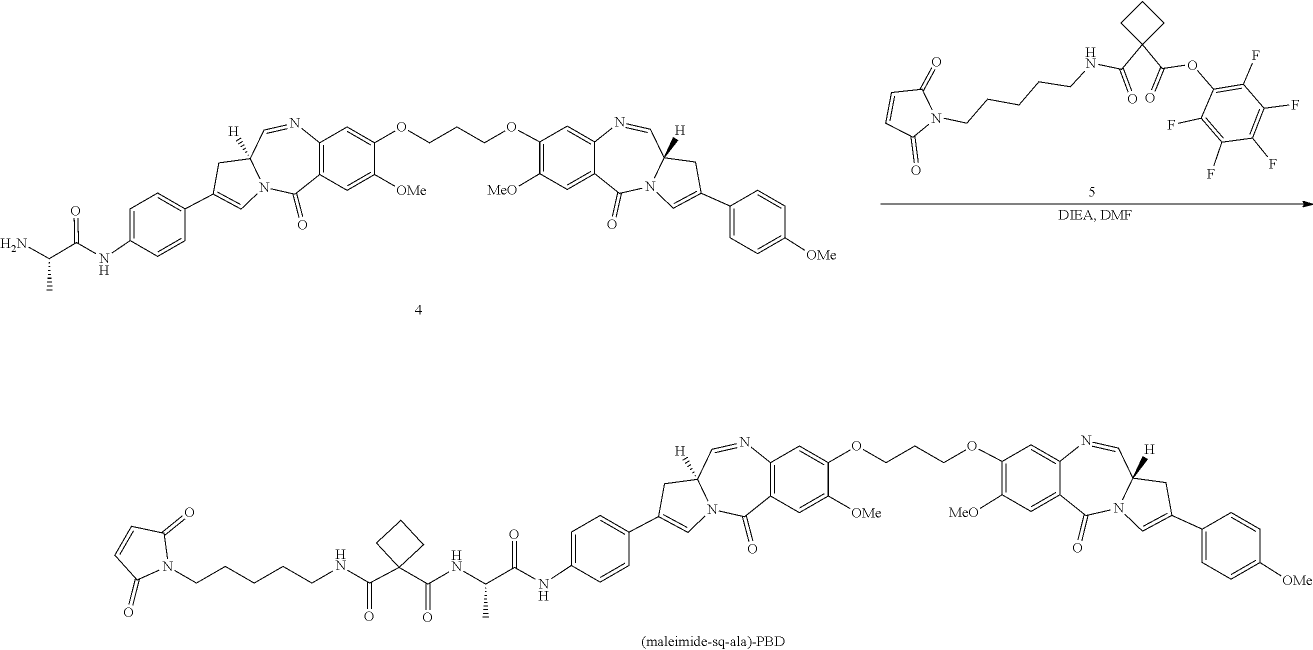 [SMILES: COc1ccc(C2=CN3C(=O)c4cc(OC)c(OCCCOc5cc6c(cc5OC)C(=O)N5C=C(c7ccc(NC(=O)[C@H](C)N)cc7)C[C@H]5C=N6)cc4N=C[C@@H]3C2)cc1 ; O=C1C=CC(=O)N1CCCCCNC(=O)C1(C(=O)Oc2c(F)c(F)c(F)c(F)c2F)CCC1 ; COc1ccc(C2=CN3C(=O)c4cc(OC)c(OCCCOc5cc6c(cc5OC)C(=O)N5C=C(c7ccc(NC(=O)[C@H](C)NC(=O)C8(C(=O)NCCCCCN9C(=O)C=CC9=O)CCC8)cc7)C[C@H]5C=N6)cc4N=C[C@@H]3C2)cc1]

To a solution of compound 5 (47.6 mg, 0.1 mmol) in DMF (2 mL) was added compound 4 (40.0 mg, 0.05 mmol) and N,N-Diisopropylethylamine (0.02 mL, 0.1 mmol). The reaction mixture was stirred at 20° C. for 2 hours. The mixture was purified by Pre-HPLC (acetonitrile: 40%-70%/0.225% FA in water) to give the product linker-payload X (25 mg, 46%) as a yellow solid. LCMS (5-95, AB, 1.5 min): RT=0.838 min, m/z=[M+H]⁺ 1087.5.

### General Procedure for Preparation of Compound 8:

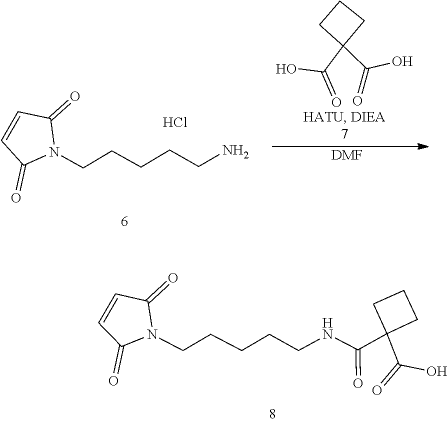

To the mixture of cyclobutane-1,1-dicarboxylic acid (0.82 g, 5.72 mmol) in DMF (10 mL) was added HATU (2.17 g, 5.72 mmol) and N,N-Diisopropylethylamine (2.22 g, 17.15 mmol), followed by addition of 1-(5-aminopentyl)pyrrole-2,5-dione hydrochloride (1.0 g, 4.57 mmol) at 0° C. The mixture was stirred at 0° C. for 1 hour. LCMS showed the reaction was completed. The solvent was removed in vacuo and dissolved in EtOAc (50 mL), then washed with aq HCl (60 mL, 1 mol/L) and brine (30 mL). The organic phase was dried over Na2SO4, filtered, and concentrated. The residue was purified by chromatography on silica eluting with 0-5% methyl alcohol in DCM to afford 1-[5-(2,5-dioxopyrrol-1-yl)pentylcarbamoyl]cyclobutanecarboxylic acid (0.90 g, 64%) as a white solid. LCMS: (5-95, AB, 1.5 min), RT=0.688 min, m/z=308.9 [M+H]+; ¹H NMR (400 MHz, CDCl₃): δ 6.71 (s, 2H), 6.37 (br s, 1H), 3.53 (t, J=6.8 Hz, 2H), 3.34-3.31 (m, 2H), 2.68-2.66 (m, 2H), 2.54-2.51 (m, 2H), 2.11-1.98 (m, 2H), 1.64-1.57 (m, 4H), 1.32-1.28 (m, 2H).

### General Procedure for Preparation of Compound 5:

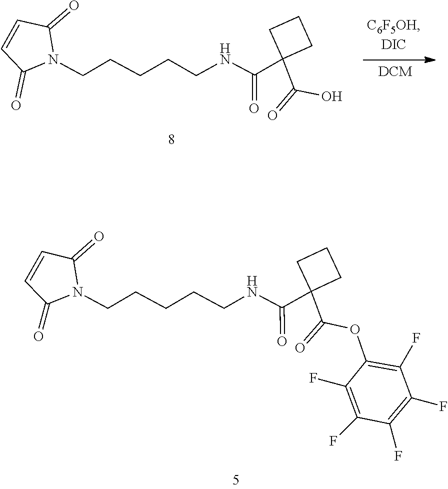 [SMILES: O=C1C=CC(=O)N1CCCCCNC(=O)C1(C(=O)O)CCC1 ; O=C1C=CC(=O)N1CCCCCNC(=O)C1(C(=O)Oc2c(F)c(F)c(F)c(F)c2F)CCC1]

Compound 8 (37.0 mg, 0.12 mmol) was added to a solution of pentafluorophenol (33 mg, 0.18 mmol) and DIC (22.7 mg, 0.18 mmol) in DCM (5 mL) at 0° C. The mixture was allowed to 20° C., and stirred for 4 hours. Then the solvent was removed under vacuum and EtOAc (1.5 mL) was added to the mixture and the resulting precipitate was removed by filtration, and the filtrate was concentrated to afford compound 5 (50 mg, 69%) as colorless oil. LCMS (5-95, AB, 1.5 min): RT=0.785 min, m/z=475.1 [M+H]+.

### Example 9—Characterization of Anti-Ly6E as an SGD-1882 Antibody-Drug Conjugate in Vitro

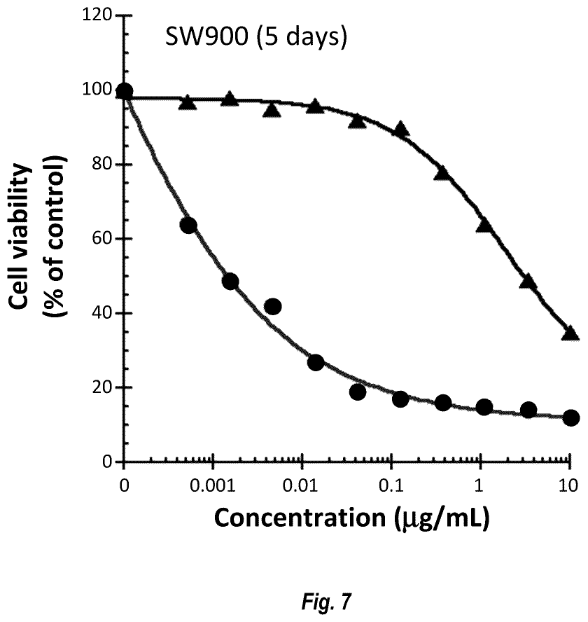

Anti-Ly6E was conjugated to SGD-1882 essentially as described above, using a maleimide-sq-ala linker with a 5-carbon spacer between the maleimide and sq. All conjugates were generated with less than 5% aggregate and at DAR>1.8. Cells were seeded in 384-well plates, grown for 24 h, and treated with the indicated ADCs including a previously described anti-Ly6E antibody (9B12). After 5 days of continuous ADC incubation, the cell viability was determined using Promega CellTiter-Glo® luminescent reagent. The luminescent intensity was measured using a PerkinElmer EnVision® reader (FIG. 7). The relative cell viability was calculated by normalizing to non-drug treatment control. Anti-Ly6E shows more potent killing against the SW900 cell line than 9B12.

### Example 9—Characterization of Anti-Ly6E as an SGD-1882 Antibody-Drug Conjugate (ADC) In Vivo

The efficacy of the Ly6E antibody drug conjugates was investigated in a human breast cancer xenograft model, HCC1569X2. The HCC1569×2 cell line was derived at Genentech from parental HCC1569 cells (ATCC) to provide optimal tumor growth in mice. This cell line was authenticated by short tandem repeat (STR) profiling using the Promega PowerPlex® 16 System and compared with external STR profiles of cell lines to determine cell line ancestry. Animal studies using this cell line were carried out at Genentech in compliance with National Institutes of Health guidelines for the care and use of laboratory animals and were approved by the Institutional Animal Care and Use Committee (IACUC) at Genentech. To establish the xenograft model, five million tumor cells (suspended in 0.2 mL of HBSS with Matrigel) were inoculated into the thoracic mammary fat pad of female C.B-17 SCID-beige mice (Charles River Laboratory; Hollister, CA).

When tumors reached the desired volume (˜250 mm³), animals were divided into groups of n=5 with similar distribution of tumor volumes, and received intravenous dose(s) of vehicle (20 mM histidine acetate, 240 mM sucrose, 0.02% polysorbate-20, pH 5.5) or antibody-drug conjugates (ADCs) through the tail vein. The treatment information was not blinded during measurement. Tumors were measured in two dimensions (length and width) using calipers and tumor volume was calculated using the formula: Tumor size (mm3)=0.5×(length×width×width). Changes in body weights were reported as a percentage relative to the starting weight. Tumor sizes and mouse body weights were recorded twice weekly over the course of the study. Mice whose tumor volume exceeded 2000 mm³ or whose body weight loss was 20% of their starting weight were promptly euthanized per IACUC guidelines.

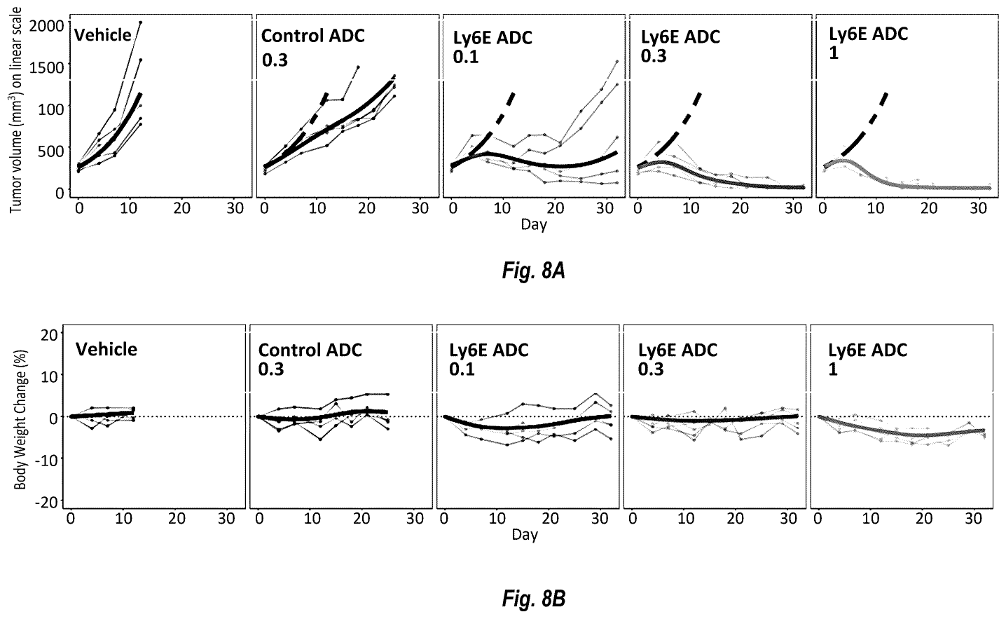

Data were analyzed using R statistical software system (R Foundation for Statistical Computing; Vienna, Austria), and a mixed modeling was fit within R using the nlme package (Pinheiro et al., (2013) Nlme: Linear and Nonlinear Mixed-Effects Models. R Package Version 31-110.3.1-113). Cubic regression splines were used to fit a non-linear profile to the time courses of body weight change or log 2 tumor volume at each dose level. These non-linear profiles were then related to dose within the mixed model. This approach addresses both repeated measurements and modest dropouts due to any non-treatment-related removal of animals before study end. Results were plotted in natural scale as fitted body weight change or tumor volume of each group over time (FIGS. 8A-8B).

### Example 10—Characterization of Anti-Ly6E as a T-Cell Engaging Bispecific

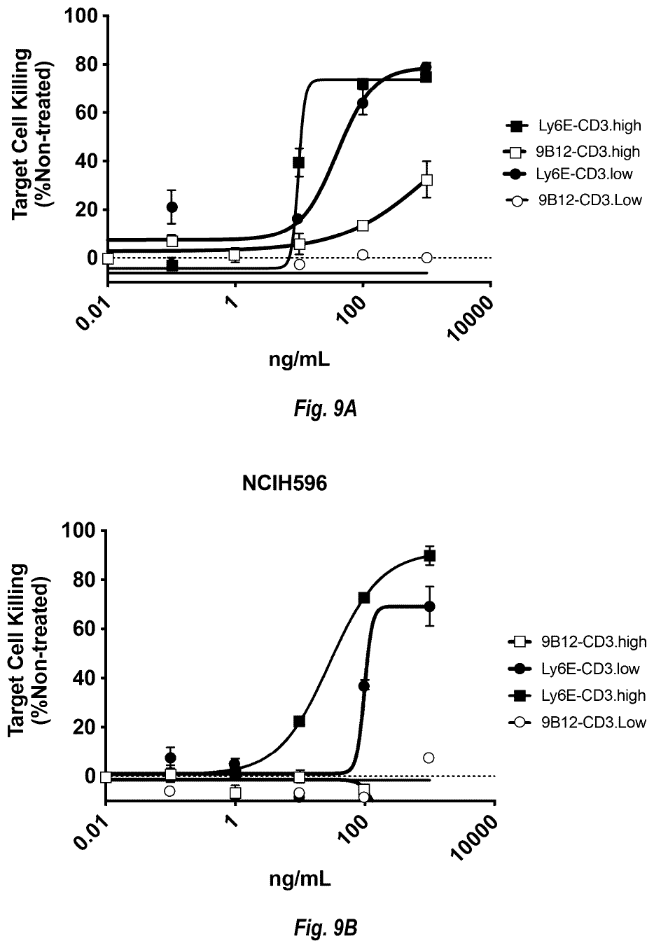

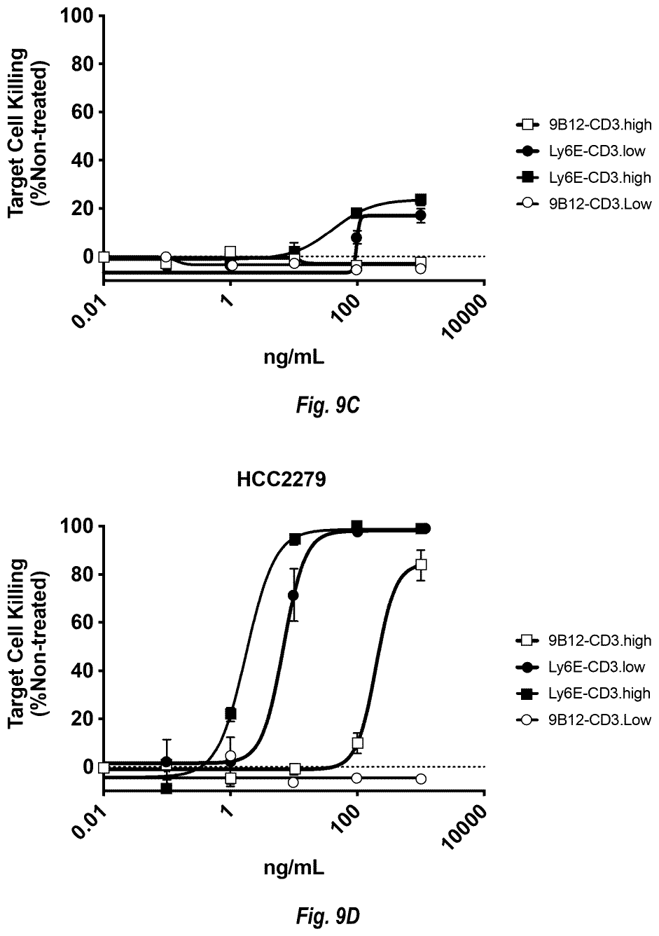

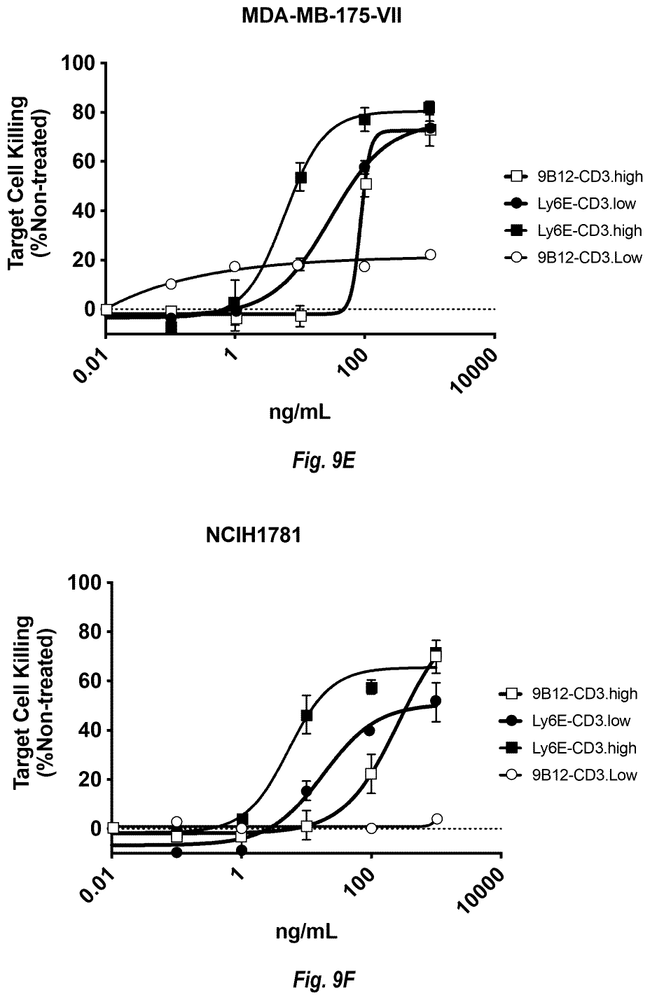

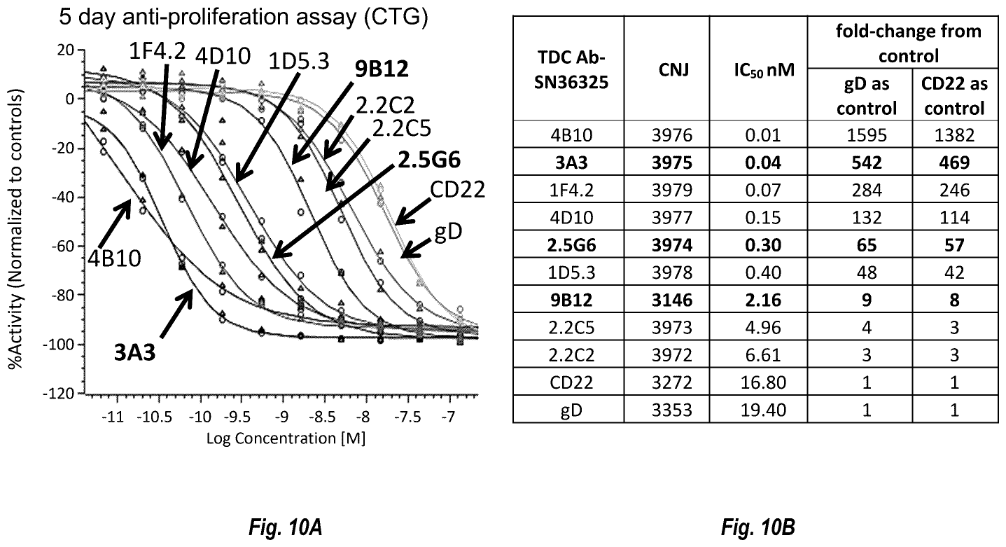

Bispecific antibodies targeting Ly6E and CD3 with high or low affinity arms were expressed using the knob-into-holes essentially as described. See Mandikian et al., Molecular cancer therapeutics 2018, 17:776-85; Atwell et al., J Mol Biol 1997, 270:26-35; and Ridgway et al., Protein Eng Des Sel 1996, 9:617-21. Cell killing induced by the bispecific antibodies was assessed essentially as previously described (see, e.g., Junttila et al., Canc. Res. 2014, 74:5561-5571) using a 4:1 effector to target ratio with CD8+ T-cells and coculturing cells for 3 days (FIGS. 9A-9F). As expected, higher affinity anti-CD3 arms corresponded to more activity. Anti-Ly6E proved superior to 9B12 across all tested cell lines.

### Example 11—Further Characterization of Anti-Ly6E as a SN36325 Antibody-Drug Conjugate

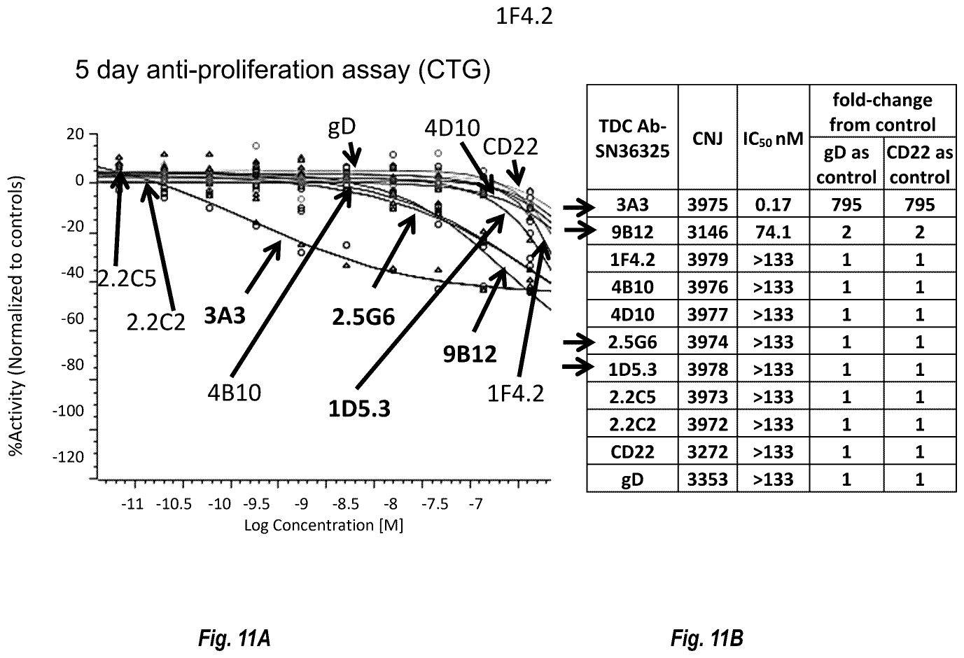

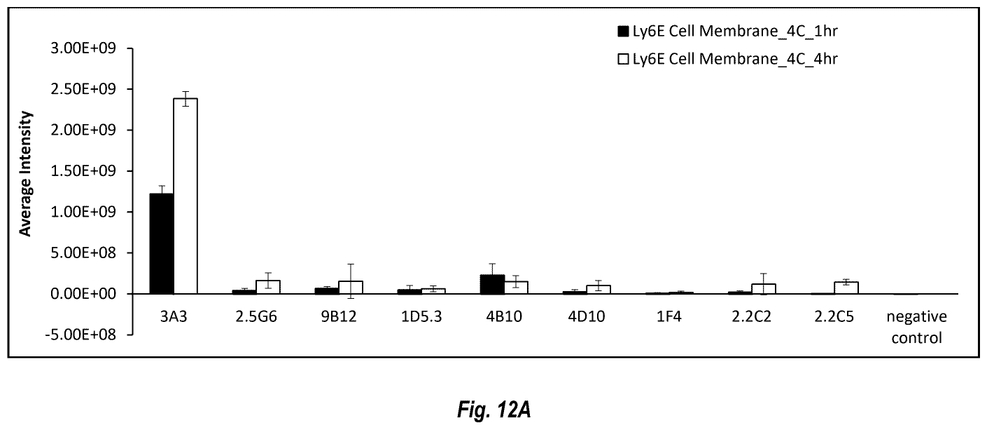

Cells were seeded in 384-well plates, grown for 24 h, and treated with the indicated ADCs including conjugates to isotype controls (anti-gD, and anti-CD22) and 9B12. After 5 days of continuous ADC incubation, the cell viability was determined using Promega CellTiter-Glo® luminescent reagent. The luminescent intensity was measured using a PerkinElmer EnVision® reader. The relative cell viability was calculated by normalizing to non-drug treatment control. Anti-Ly6E conjugates (3A3 and 4B10) show more potent killing against both NCl—H1781 (FIGS. 10A-10B) and HCC1569x2 (FIGS. 11A-11B) cell lines than isotype control, 9B12, and other ADC conjugates. For example, FIGS. 10A-10B show that chimeric anti-Ly6E 4B10 and 3A3 show >400-fold potency difference from non-target control (e.g., isotype controls anti-gD and anti-CD22) compared to only 9-fold with 9B12 in cell line NCl—H1781. FIGS. 11A-11B show that chimeric anti-Ly6E 3A3 shows the highest potency against cell line HCC1569x2, compared to other ADCs and control-conjugates, despite overall weaker potency with partial killing.

### Example 12—Characterization of 3A3 Binding

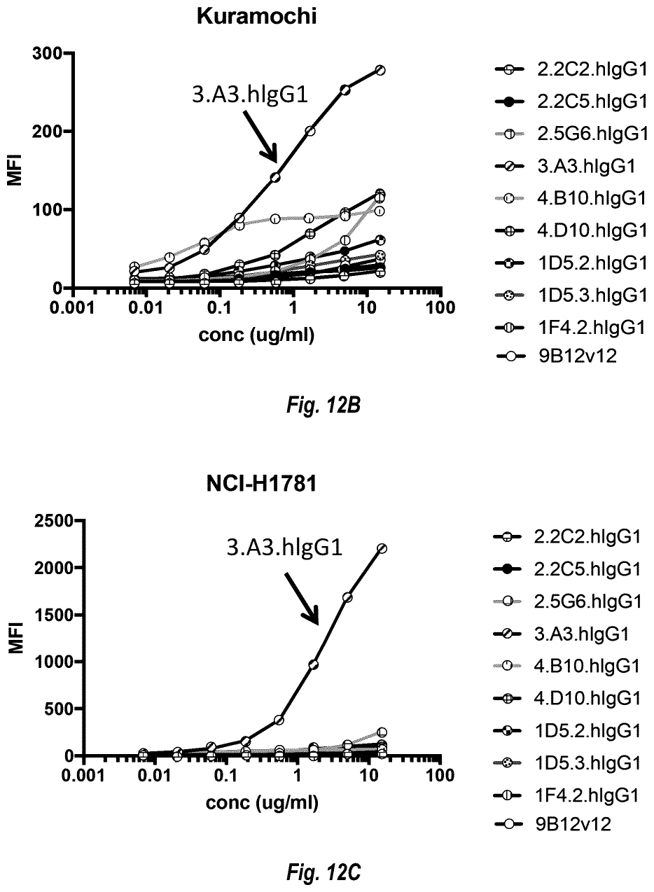

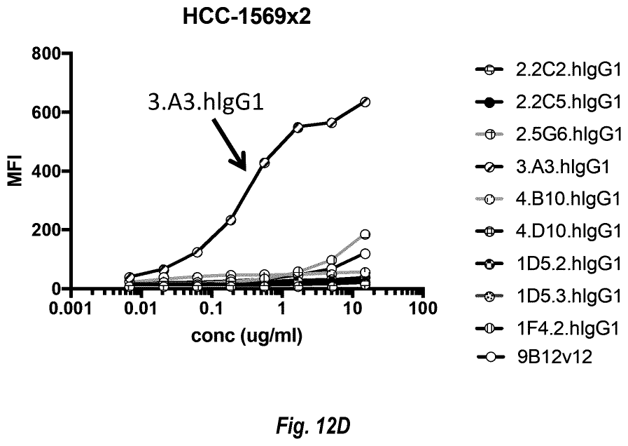

On Day 0, Kuramochi cells were seeded in 384-well Cell Carrier plates (Perkin Elmer) and allowed to adhere overnight to attain an 80% confluency. The next day, cells were treated with unconjugated antibodies at 2 and 20 μg/mL with cold media containing 10 μg/mL leupeptin and 5 μM pepstatin protease inhibitors. Antibodies were allowed to bind for 1 hour on ice. Cells with pulse treatments were washed with cold media containing protease inhibitors, while cells with continuous treatment were left alone. Cells were incubated at 37° C., 5% humidified CO₂ for 1 hour and 3 hours for antibody internalization. After incubation, cells were fixed with 4% paraformaldehyde (PFA)/4% sucrose in phosphate-buffered saline (PBS) for 10 minutes at room temperature (RT), and then cells were washed six times with 1×PBS. Cells were blocked and permeabilized with 2% donkey serum containing 0.05% saponin (block buffer) for 1 hour. Block buffer was removed, then rabbit anti-LAMP1 (Sigma Aldrich, L1418) primary antibody in block buffer was added and incubated overnight. The next day, cells were washed six times with PBS containing 0.05% saponin, and then secondary antibodies donkey anti-human IgG-488. Only anti-Ly6E proved showed substantially enhanced binding to Ly6E over-expressing cells. See FIG. 12A.

Further, direct binding of antigen-binding fragment (Fab)-luciferase fusions to three different Ly6E-expressing cells was assessed following a 4 hour incubation at 4° C. FIG. 12B-12D show that 3A3 (anti-ly6E) has substantially enhanced binding in Kuramochi (FIG. 12B), NCl—H1781 (FIG. 12C), and HCC-1569x2 (FIG. 12D) cell lines validated to express Ly6E by Western blot. Points represent the average of two replicate samples. BIAcore™ Fab binding (Kd=nM) was also shown for 3A3(218 Kd)>2.2C2(168 Kd)>2.2C5(63 Kd)>1D5.3(20 Kd)>2.5G6(14 Kd)>1D5.2(12 Kd).

### Example 13—Colocalization

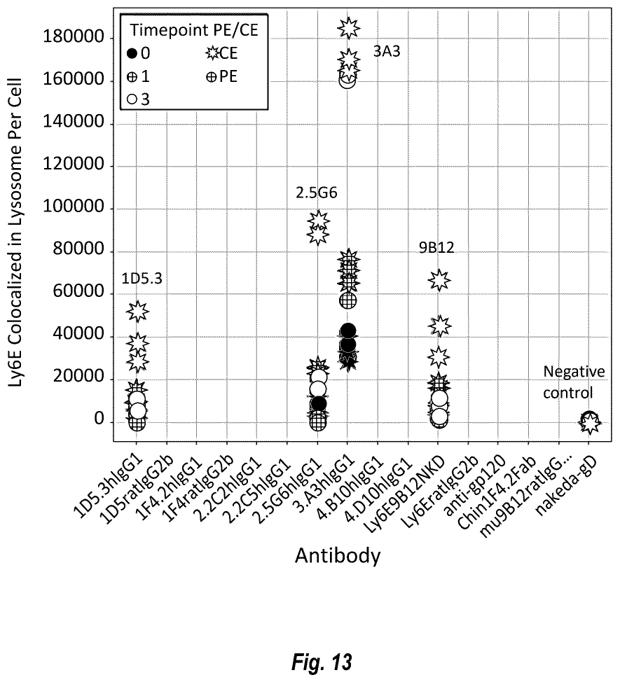

Kuramochi cells were exposed to each antibody at 2 μg/mL for 1 hour at 4° C., and antibodies were either washed for pulse exposure (PE) or not washed for continuous exposure (CE), then colocalization and internalization (intensity in cells/intensity in +out cells) was measured at 0, 1, and 3 hours at 37° C. FIG. 13 shows that the amount of 3A3 colocalization is significantly higher compared to antibodies 1D5.3, 2.5G6, 9B12, and control.

### Example 14—Internalization

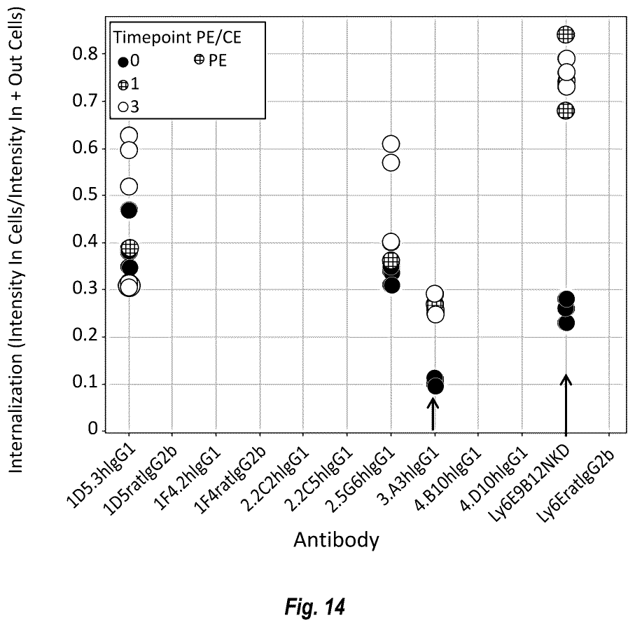

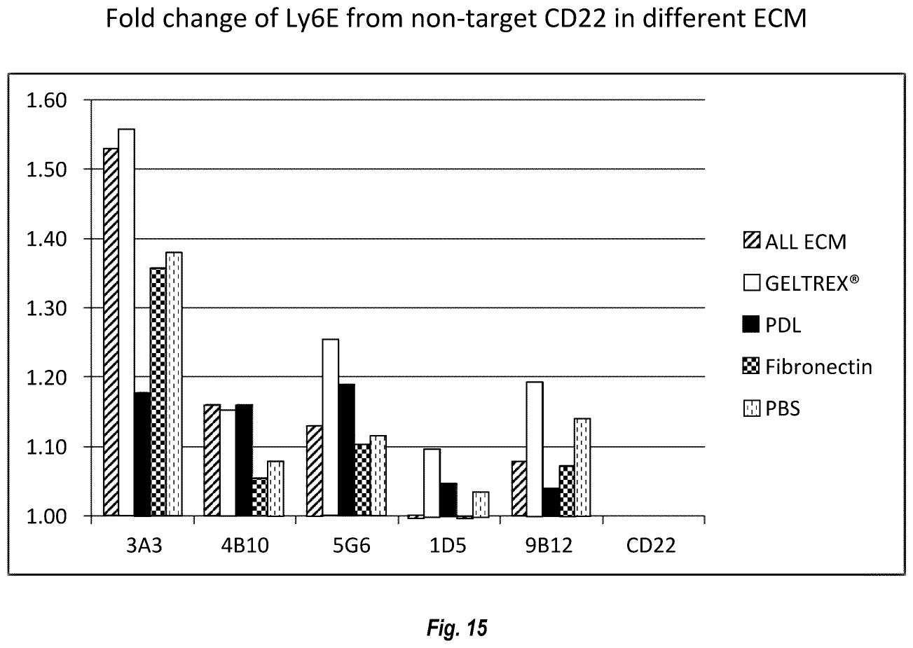

To assess internalization rates, a pulse-chase experiment was performed where antibodies were incubated with Kuramochi cells at 2 μg/mL for 1 hour at 4° C., washed, and internalization was monitored over time (0, 1, and 3 hours). FIG. 14 shows that the rate of internalization for 9B12 was faster compared to 3A3, but the higher binding of 3A3 leads to durable efficacy as seen in in vivo killing studies.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An isolated antibody that binds to Ly6E, wherein the antibody comprises (i) HVR-L1 comprising the amino acid sequence of SEQ ID NO:7, (ii) HVR-L2 comprising the amino acid sequence of SEQ ID NO:8, (iii) HVR-L3 comprising the amino acid sequence of SEQ ID NO:9, (iv) HVR-H1 comprising the amino acid sequence of SEQ ID NO:10, v) HVR-H2 comprising the amino acid sequence of SEQ ID NO:11, and (vi) HVR-H3 comprising the amino acid sequence of SEQ ID NO: 12.

2. The antibody of claim 1, wherein the antibody is a monoclonal antibody.

3. The antibody of claim 1 wherein the antibody is a humanized or chimeric antibody.

4. The antibody of claim 1, wherein the antibody is an antibody fragment.

5. The antibody of claim 1, comprising (a) a VH sequence having at least 95% sequence identity to SEQ ID NO:32; (b) a VL sequence having at least 95% sequence identity to the amino acid sequence of SEQ ID NO:31; or (c) a VH sequence as in (a) and a VL sequence as in (b).

6. The antibody of claim 5, comprising a VH sequence of SEQ ID NO:32 and a VL sequence of SEQ ID NO: 31.

7. (canceled)

8. The antibody of claim 1, wherein the antibody is an IgG1, IgG2a, IgG2b, IgG3, or IgG4 antibody.

9. The antibody of claim 1, wherein the antibody comprises a heavy chain comprising an amino acid sequence of SEQ ID NO: 5 and a light chain comprising an amino acid sequence selected from SEQ ID NO:3, SEQ ID NO:17, SEQ ID NO: 19, or SEQ ID NO: 29.

10. (canceled)

11. An isolated antibody that binds to Ly6E, wherein the antibody comprises a heavy chain comprising the amino acid sequence of SEQ ID NO: 5 and a light chain comprising the amino acid sequence of SEQ ID NO: 3.

12. The antibody of claim 1, wherein the antibody is a multispecific antibody.

13. (canceled)

14. The antibody of claim 12, wherein the multispecific antibody binds Ly6E and CD3.

15. An isolated nucleic acid encoding the antibody of claim 1.

16. An expression vector comprising the nucleic acid of claim 15.

17. A host cell comprising the nucleic acid of claim 14.

18. A host cell that expresses the antibody of claim 1.

19. A method of producing an antibody comprising culturing the host cell of claim 18 so that the antibody is produced.

20. An immunoconjugate comprising the antibody of claim 1 and a cytotoxic agent.

21. The immunoconjugate of claim 20 having the formula Ab-(L-D)ₚ, wherein:
(a) Ab is an antibody comprising (i) HVR-L1 comprising the amino acid sequence of SEQ ID NO:7, (ii) HVR-L2 comprising the amino acid sequence of SEQ ID NO:8, (iii) HVR-L3 comprising the amino acid sequence of SEQ ID NO:9, (iv) HVR-H1 comprising the amino acid sequence of SEQ ID NO:10, v) HVR-H2 comprising the amino acid sequence of SEQ ID NO:11, and (vi) HVR-H3 comprising the amino acid sequence of SEQ ID NO:12;
(b) L is a linker;
(c) D is a pyrrolobenzodiazepine; and
(d) p ranges from 1-8.

22. The immunoconjugate of claim 21, wherein D is a pyrrolobenzodiazepine of Formula A: 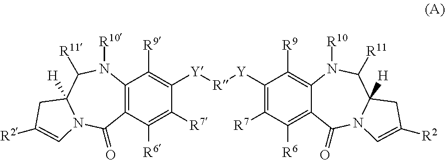
wherein:
R² is of formula II: 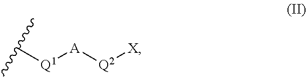
wherein A is a C₅₋₇ aryl group, X is selected from: OH, SH, CO₂H, COH, N═C═O, NHR^{N}, and (OC₂H₄)ₘOCH₃, wherein R^{N} is selected from H and C₁₋₄ alkyl, and wherein m is an integer from 1 to 3, and either:
(i) Q¹ is a single bond, and Q² is selected from a single bond and —Z—(CH₂)ₙ—, wherein Z is a single bond, O, S, or NH and n is an integer from 1 to 3; or
(ii) Q¹ is —CH═CH—, and Q² is a single bond;
R^{2′} is a C₅₋₁₀ aryl group, non-substituted or substituted by one or more substituents selected from halo, nitro, cyano, ether, C₁₋₇ alkyl, C₃₋₇ heterocyclyl, and bis-oxy-C₁₋₃ alkylene;
R⁶ and R⁹ are independently selected from H, R, OH, OR, SH, SR, NH₂, NHR, NRR′, nitro,
Me₃Sn and halo;
R⁷ is selected from the H, R, OH, OR, SH, SR, NH₂, NHR, NHRR′, nitro, Me₃Sn, and halo;
where R and R′ are independently selected from non-substituted or substituted C₁₋₁₂ alkyl, C₃₋₂₀ heterocyclyl and C₅₋₂₀ aryl groups;
either:
(a) R¹⁰ is H, and R¹¹ is OH or OR^{A}, wherein R^{A} is C₁₋₄ alkyl;
(b) R¹⁰ and R¹¹ form a nitrogen-carbon double bond between the nitrogen and carbon atoms to which they are bound; or
(c) R¹⁰ is H and R¹¹ is SO_{z}M, wherein z is 2 or 3 and M is a monovalent pharmaceutically acceptable cation;
R″ is a C₃₋₁₂ alkylene group, which chain may be interrupted by one or more heteroatoms independently selected from O, S, and NH, and/or one or more aromatic rings independently selected from benzene or pyridine;
Y is O, S, or NH;
R^{6′}, R^{7′}, and R^{9′} are selected from the same groups as R⁶, R⁷ and R⁹ respectively, and R^{10′} and R^{11′} are the same as R¹⁰ and R¹¹ respectively, wherein if R¹¹ and R^{11′} are SO_{z}M, M may represent a divalent pharmaceutically acceptable cation; and
the point of attachment to the linker L is through R² or R^{2′}.

23. The immunoconjugate of claim 22, wherein D has the structure: 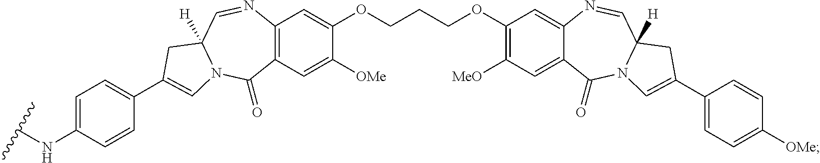 [SMILES: *Nc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]
wherein the squiggly line indicates the point of attachment to the linker L.

24. The immunoconjugate of claim 21, wherein:
(a) (L-D) has the following structure: 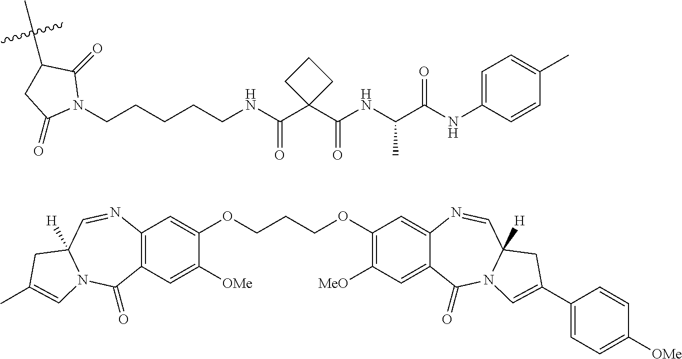
wherein the squiggly line indicates the point of attachment to the protein.

25. The immunoconjugate of claim 21, wherein p ranges from 1.5-6.

26. (canceled)

27. (canceled)

28. A pharmaceutical formulation comprising the immunoconjugate of claim 20 and a pharmaceutically acceptable carrier.

29. The pharmaceutical formulation of claim 28, further comprising an additional therapeutic agent.

30. A method of treating a subject having a Ly6E-positive cancer, the method comprising administering to the subject an effective amount of the immunoconjugate of claim 20.

31. The method of claim 30, wherein the Ly6E-positive cancer is breast cancer, pancreatic cancer, colon cancer, colorectal cancer, melanoma, ovarian cancer, non-small cell lung cancer, or gastric cancer.

32. The method of claim 30, wherein the method further comprises administering an additional therapeutic agent to the individual.

33. The method of claim 32, wherein the additional therapeutic agent is a platinum complex.

34. A method of inhibiting proliferation of a Ly6E-positive cell, the method comprising exposing the cell to the immunoconjugate of claim 20 under conditions permissive for binding of the immunoconjugate to Ly6E on the surface of the cell, thereby inhibiting proliferation of the cell.

35. The method of claim 34, wherein the cell is a breast, pancreatic, colon, colorectal, melanoma, ovarian non-small cell lung or gastric cancer cell.

36. The antibody of claim 1 conjugated to a label.

37. The antibody of claim 36, wherein the label is a positron emitter.

38. The antibody of claim 37, wherein the positron emitter is ⁸⁹Zr.

39. A method of detecting human Ly6E in a biological sample comprising contacting the biological sample with the anti-Ly6E antibody of claim 1 under conditions permissive for binding of the anti-Ly6E antibody to a naturally occurring human Ly6E, and detecting whether a complex is formed between the anti-Ly6E antibody and a naturally occurring human Ly6E in the biological sample.

40. (canceled)

41. The method of claim 39, wherein the biological sample is a breast cancer sample, a pancreatic cancer sample, a colon cancer sample, a colorectal cancer sample, melanoma cancer sample, ovarian cancer sample, a non-small cell lung cancer sample, or a gastric cancer sample.

42. A method for detecting a Ly6E-positive cancer comprising: (i) administering a labeled anti-Ly6E antibody to a subject having or suspected of having a Ly6E-positive cancer, wherein the labeled anti-Ly6E antibody comprises the anti-Ly6E antibody of claim 1; and (ii) detecting the labeled anti-Ly6E antibody in the subject, wherein detection of the labeled anti-Ly6E antibody indicates a Ly6E-positive cancer in the subject.

43. (canceled)

44. (canceled)

45. (canceled)


SEQUENCE LISTING (34 sequences)

SEQ ID NO: 1 - protein, 131 aa, Homo sapiens
MKIFLPVLLAALLGVERASSLMCFSCLNQKSNLYCLKPTICSDQDNYCVTVSASAGIGNLVTFGHSLSKTCSPACPIPEGVNVGVASMGISCCQSFLCNFSAADGGLRASVTLLGAGLLLSLLPALLRFGP

SEQ ID NO: 2 - protein, 131 aa, Macaca sp.
MKIFLPVLLAALLGVERASSLMCFSCLNQKSNLYCLKPTICSDQDNYCVTVSTSAGIGNLVTFGHSLSKTCSPACPLPEGINVGVASMGISCCQSFLCNFSAADGGLRASATLLGAGLLLSLLPALLRFGP

SEQ ID NO: 3 - protein, 213 aa, synthetic construct
  source 1..213: note = Humanized light chain amino acid sequence of anti-Ly6E antibody clone 3A3.v2.D92E
DIQMTQSPSSLSASVGDRVTITCKASQNVGSNVDWYQQKPGKAPKLLIYKASNRYTGVPSRFSGSGSGTDFTFTISSLQPEDIATYYCMQSETKYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 213 aa, synthetic construct
  source 1..213: note = Chimeric variable light chain amino acid sequence of anti-Ly6E antibody clone 3A3
DIVMTQSPTSISISVGERVTMNCKASQNVGSNVDWYQQKTGQSPELLIYKASNRYTGVPGRFTGSGSGTDFTFTISNLQAEDLAVYYCMQSDTKYTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 453 aa, synthetic construct
  source 1..453: note = Humanized heavy chain amino acid sequence of anti-Ly6E antibody clone 3A3
EVQLVESGGGLVQPGGSLKLSCAASGFTFSTVAMHWVRQASGKGLEWVGRIRTKPVNYETYYADSVKGRFTISRDDSKSTVYLQMNSLKTEDTAVYYCSAPNYYDGTYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 453 aa, synthetic construct
  source 1..453: note = Chimeric heavy chain amino acid sequence of anti-Ly6E antibody clone 3A3
AVQLVEFGGGLVQPKESLKISCAASGFTFSTVAMHWVRQAPGKGLEWIARIRTKPVNYETYYADSVKGRFTISRDDSKSMVYLQMDNLKTEDAAMYYCSAPNYYDGTYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 11 aa, synthetic construct
  source 1..11: note = Humanized variable light chain CDR 1 amino acid sequence of anti-Ly6E antibody clone 3A3.v2.D92E
KASQNVGSNVD

SEQ ID NO: 8 - protein, 7 aa, synthetic construct
  source 1..7: note = Humanized variable light chain CDR 2 amino acid sequence of anti-Ly6E antibody clone 3A3.v2.D92E
KASNRYT

SEQ ID NO: 9 - protein, 8 aa, synthetic construct
  source 1..8: note = Humanized variable light chain CDR 3 amino acid sequence of anti-Ly6E antibody clone 3A3.v2.D92E
MQSETKYT

SEQ ID NO: 10 - protein, 10 aa, synthetic construct
  source 1..10: note = Humanized variable heavy chain CDR 1 amino acid sequence of anti-Ly6E antibody clone 3A3
GFTFSTVAMH

SEQ ID NO: 11 - protein, 19 aa, synthetic construct
  source 1..19: note = Humanized variable heavy chain CDR 2 amino acid sequence of anti-Ly6E antibody clone 3A3
RIRTKPVNYETYYADSVKG

SEQ ID NO: 12 - protein, 14 aa, synthetic construct
  source 1..14: note = Humanized variable heavy chain CDR 3 amino acid sequence of anti-Ly6E antibody clone 3A3
SAPNYYDGTYSFAY

SEQ ID NO: 13 - DNA, 1419 bp, synthetic construct
  source 1..1419: mol_type = other DNA; note = Humanized heavy chain nucleotide sequence of anti-Ly6E antibody clone 3A3 with signal sequence
atgggatggtcatgtatcatcctttttctagtagcaactgcaaccggtgtacattcagaggtgcagctggtggagtctgggggaggcttggtccagcctggggggtccctgaaactctcctgtgcagcctctgggttcaccttcagtaccgttgctatgcactgggtccgccaggcttccgggaaagggctggagtgggttggccgtattagaaccaaacctgtcaattacgagacatactatgctgactcggtgaaaggcaggttcaccatctccagagatgattcaaagtccacggtgtatctgcaaatgaacagcctgaaaaccgaggacacggccgtgtattactgttctgcaccaaattactacgacggtacttactcttttgcctactggggccaaggaaccctggtcaccgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - DNA, 699 bp, synthetic construct
  source 1..699: mol_type = other DNA; note = Humanized light chain nucleotide sequence of anti-Ly6E antibody clone 3A3.v2.D92E with signal sequence
atgggatggtcatgtatcatcctttttctagtagcaactgcaaccggtgtacattcagacatccagatgacccagtctccatcctccctgtctgcatctgtaggagacagagtcaccatcacttgcaaggcgagtcagaacgttggctccaatgtagattggtatcagcagaaaccagggaaagcccctaagctcctgatctacaaggcatccaatcggtacacaggggtcccatcaaggttcagtggaagtggatctgggacagattttactttcaccatcagcagcctgcagcctgaagatattgcaacatattactgtatgcagtctgagactaagtacacttttggccagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 15 - DNA, 699 bp, synthetic construct
  source 1..699: mol_type = other DNA; note = Chimeric light chain nucleotide sequence of anti-Ly6E antibody clone 3A3 with signal sequence
atgggatggtcatgtatcatcctttttctagtagcaactgcaaccggtgtacattcagacattgtgatgactcagtctcccacatccatttccatatcagtaggagagagggtcaccatgaactgcaaggccagtcagaatgtgggttctaatgttgactggtatcaacagaaaacagggcagtctcctgaactgcttatctacaaggcatccaatcggtacactggcgtccctggtcgcttcacaggcagtggatctggaacagatttcactttcaccatcagcaacttgcaggctgaagacctggctgtttattactgtatgcagtctgacaccaagtacacgtttggagctggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 16 - DNA, 1419 bp, synthetic construct
  source 1..1419: mol_type = other DNA; note = Chimeric heavy chain nucleotide sequence of anti-Ly6E antibody clone 3A3 with signal sequence
atgggatggtcatgtatcatcctttttctagtagcaactgcaaccggtgtacattcagcggtacaacttgttgagtttggtggaggattggtgcagcctaaggagtcattgaaaatctcatgtgcagcctctggattcaccttcagtactgttgccatgcactgggtccgccaggctccaggaaagggtctggaatggattgctcgcattagaactaaacctgttaattatgaaacatattatgctgattcagtgaaaggcagattcaccatctcccgagatgattcaaagagcatggtctacctacaaatggataacttgaaaactgaggacgcagccatgtattactgttcagctcctaattattatgatggtacttattcctttgcttactggggccaaggcactctggtaactgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 17 - protein, 213 aa, synthetic construct
  source 1..213: note = Humanized light chain amino acid sequence of anti-Ly6E antibody clone 3A3
DIQMTQSPSSLSASVGDRVTITCKASQNVGSNVDWYQQKPGKSPKLLIYKASNRYTGVPSRFSGSGSGTDFTFTISSLQPEDIATYYCMQSDTKYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 698 bp, synthetic construct
  source 1..698: mol_type = other DNA; note = Humanized light chain nucleotide sequence of anti-Ly6E antibody clone 3A3 with signal sequence
atgggatggtcatgtatcatcctttttctagtagcaactgcaaccggtgtacattcagacatccagatgacccagtctccatcctccctgtctgcatctgtaggagacagagtcaccatcacttgcaaggcgagtcagaacgttggctccaatgtagattggtatcagcagaaaccagggaaatcccctaagctcctgatctacaaggcatccaatcggtacacaggggtcccatcaaggttcagtggaagtggatctgggacagattttactttcaccatcagcagcctgcagcctgaagatattgcaacatattactgtatgcagtctgatactaagtacacttttggccagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtta

SEQ ID NO: 19 - protein, 213 aa, synthetic construct
  source 1..213: note = Humanized light chain amino acid sequence of anti-Ly6E antibody clone 3A3.K149C
DIQMTQSPSSLSASVGDRVTITCKASQNVGSNVDWYQQKPGKSPKLLIYKASNRYTGVPSRFSGSGSGTDFTFTISSLQPEDIATYYCMQSDTKYTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 20 - DNA, 699 bp, synthetic construct
  source 1..699: mol_type = other DNA; note = Humanized light chain nucleotide sequence of anti-Ly6E antibody clone 3A3.K149C with signal sequence
atgggatggtcatgtatcatcctttttctagtagcaactgcaaccggtgtacattcagacatccagatgacccagtctccatcctccctgtctgcatctgtaggagacagagtcaccatcacttgcaaggcgagtcagaacgttggctccaatgtagattggtatcagcagaaaccagggaaatcccctaagctcctgatctacaaggcatccaatcggtacacaggggtcccatcaaggttcagtggaagtggatctgggacagattttactttcaccatcagcagcctgcagcctgaagatattgcaacatattactgtatgcagtctgatactaagtacacttttggccagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggtgcgtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 21 - protein, 131 aa, Macaca mulatta; identical to SEQ ID NO: 2

SEQ ID NO: 22 - protein, 136 aa, Mus sp.
MSATSNMRVFLPVLLAALLGMEQVHSLMCFSCTDQKNNINCLWPVSCQEKDHYCITLSAAAGFGNVNLGYTLNKGCSPICPSENVNLNLGVASVNSYCCQSSFCNFSAAGLGLRASIPLLGLGLLLSLLALLQLSP

SEQ ID NO: 23 - protein, 136 aa, Rattus sp.
MSAASSMRVFLPVLLAALLGVEQVHSLMCFSCTDQKNNINCLWPVSCSSTDNYCITLSAAAGFGNVNLGYTLNKGCSPTCPRENININLGVASVNSYCCQSSFCNFSTAGLGLRASIPLLGLGLLLSLLAVLRLSP

SEQ ID NO: 24 - protein, 111 aa, Homo sapiens
LMCFSCLNQKSNLYCLKPTICSDQDNYCVTVSASAGIGNLVTFGHSLSKTCSPACPIPEGVNVGVASMGISCCQSFLCNFSAADGGLRASVTLLGAGLLLSLLPALLRFGP

SEQ ID NO: 25 - protein, 111 aa, Macaca fascicularis
LMCFSCLNQKSNLYCLKPTICSDQDNYCVTVSTSAGIGNLVTFGHSLSKTCSPACPLPEGINVGVASMGISCCQSFLCNFSAADGGLRASATLLGAGLLLSLLPALLRFGP

SEQ ID NO: 26 - protein, 111 aa, Macaca mulatta; identical to SEQ ID NO: 25

SEQ ID NO: 27 - protein, 110 aa, Mus sp.
LMCFSCTDQKNNINCLWPVSCQEKDHYCITLSAAAGFGNVNLGYTLNKGCSPICPSENVNLNLGVASVNSYCCQSSFCNFSAAGLGLRASIPLLGLGLLLSLLALLQLSP

SEQ ID NO: 28 - protein, 110 aa, Rattus sp.
LMCFSCTDQKNNINCLWPVSCSSTDNYCITLSAAAGFGNVNLGYTLNKGCSPTCPRENININLGVASVNSYCCQSSFCNFSTAGLGLRASIPLLGLGLLLSLLAVLRLSP

SEQ ID NO: 29 - protein, 213 aa, synthetic construct
  source 1..213: note = Humanized light chain amino acid sequence of anti-Ly6E antibody clone 3A3.v2.D92S
DIQMTQSPSSLSASVGDRVTITCKASQNVGSNVDWYQQKPGKAPKLLIYKASNRYTGVPSRFSGSGSGTDFTFTISSLQPEDIATYYCMQSSTKYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - DNA, 728 bp, synthetic construct
  source 1..728: mol_type = other DNA; note = Humanized light chain nucleotide sequence of anti-Ly6E antibody clone 3A3.v2.D92S
atgggatggtcatgtatcatcctttttctagtagcaactgcaaccggtgtacattcagacatccagatgacccagtctccatcctccctgtctgcatctgtaggagacagagtcaccatcacttgcaaggcgagtcagaacgttggctccaatgtagattggtatcagcagaaaccagggaaagcccctaagctcctgatctacaaggcatccaatcggtacacaggggtcccatcaaggttcagtggaagtggatctgggacagattttactttcaccatcagcagcctgcagcctgaagatattgcaacatattactgtatgcagtcttctactaagtacacttttggccagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagaaacagctatgacatgattacgaattaa

SEQ ID NO: 31 - protein, 106 aa, synthetic construct
  source 1..106: note = Humanized variable light chain amino acid sequence of anti-Ly6E antibody clone 3A3.v2.D92E
DIQMTQSPSSLSASVGDRVTITCKASQNVGSNVDWYQQKPGKAPKLLIYKASNRYTGVPSRFSGSGSGTDFTFTISSLQPEDIATYYCMQSETKYTFGQGTKVEIK

SEQ ID NO: 32 - protein, 123 aa, synthetic construct
  source 1..123: note = Humanized variable heavy chain amino acid sequence of anti-Ly6E antibody clone 3A3
EVQLVESGGGLVQPGGSLKLSCAASGFTFSTVAMHWVRQASGKGLEWVGRIRTKPVNYETYYADSVKGRFTISRDDSKSTVYLQMNSLKTEDTAVYYCSAPNYYDGTYSFAYWGQGTLVTVSS

SEQ ID NO: 33 - protein, 452 aa, synthetic construct
  source 1..452: note = Humanized heavy chain amino acid sequence of anti-Ly6E antibody clone 3A3, without C-term lysine (K)
EVQLVESGGGLVQPGGSLKLSCAASGFTFSTVAMHWVRQASGKGLEWVGRIRTKPVNYETYYADSVKGRFTISRDDSKSTVYLQMNSLKTEDTAVYYCSAPNYYDGTYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 452 aa, synthetic construct
  source 1..452: note = Chimeric heavy chain amino acid sequence of anti-Ly6E antibody clone 3A3, without C-term lysine (K)
AVQLVEFGGGLVQPKESLKISCAASGFTFSTVAMHWVRQAPGKGLEWIARIRTKPVNYETYYADSVKGRFTISRDDSKSMVYLQMDNLKTEDAAMYYCSAPNYYDGTYSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG